(19) United States
(12) Patent Application Publication
(10) Pub. No.: US 2024/0052235 A1
(43) Pub. Date: Feb. 15, 2024

(54) ELECTROCHROMIC COMPOUNDS AND OPTICAL ARTICLES CONTAINING THEM

(71) Applicant: Essilor International, Charenton-Le-Pont (FR)

(72) Inventors: Claudine BIVER, Charenton-Le-Pont (FR); Fabien BERIT-DEBAT, Charenton-Le-Pont (FR); Samuel ARCHAMBEAU, Charenton-Le-Pont (FR); Stuart AIKEN, Leeds (GB); Georgina K. ARMITAGE, Leed (GB); Thomas D. BROADBENT, Leeds (GB); Daniel L. CROSSLEY, Leeds (GB); Christopher D. GABBUTT, Leeds (GB); Bernard Mark HERON, Leeds (GB)

(73) Assignee: Essilor International, Charenton-Le-Pont (FR)

(21) Appl. No.: 18/258,738

(22) PCT Filed: Dec. 14, 2021
(86) PCT No.: PCT/EP2021/085653

§ 371 Date: Jun. 21, 2023

(30) Foreign Application Priority Data
Dec. 22, 2020 (EP) 20306663.4

(51) Int. Cl. C09K 9/02; C07D 417/04; C07D 401/04; C07D 471/04; C07D 401/14; C07D 421/04; C07D 413/04

(52) U.S. Cl. CPC C09K 9/02; C07D 417/04; C07D 401/04; C07D 471/04; C07D 401/14; C07D 421/04; C07D 413/04; C09K 2211/1018; C09K 2211/1007; G02F 1/0018

(57) ABSTRACT

The invention relates to a group of novel electrochromic compounds. More specifically, it relates to benzazoles and condensed azole compounds substituted with one or several pyridinium rings and the use of these compounds as a variable transmittance medium for the manufacture of an optical article, such as an ophthalmic lens.

The present invention relates to a group of novel electrochromic compounds. More specifically, it relates to benzazoles and condensed azole compounds substituted with one or several pyridinium rings and the use of these compounds as a variable transmittance medium for the manufacture of an optical article, such as an ophthalmic lens.

Electrochromism is a well-known physical phenomenon which is observed with certain classes of chemical compounds that reversibly change colour when a voltage is applied to them. The material undergoes reversible changes in optical properties by oxidation and reduction. Advantageously, the electrochromic material is colourless when an electric field is not applied and becomes coloured when an electric field is applied.

An electrochromic device, i.e. a device containing electrochromic compounds, the absorbance of which depends only on the presence of an electric field, can thus have two states, i.e. a coloured state (when electrically activated) and a bleached state (in the inactive state). The optical transmission properties of the device depend on the nature of the electrochromic compounds.

There remains a need for improving electrochromic materials in order to use them as transparent media for forming high quality articles, in particular high quality ophthalmic lenses, while keeping electrochromic properties and having a wide range of colours.

Surprisingly, the inventors have found that introducing an azole ring or an azolium ring in the structure of the electrochromic molecules lowers their absorption wavelength in the visible range. Indeed, different azole rings have been introduced and were found to efficiently widen the wavelength coverage at various activation potentials. This was the case for instance with substituted imidazoliums and fused-ring derivatives, substituted benzimidazoliums, substituted benz(iso)thiazolium compounds. In addition, viologens molecules or benzazoles or condensed azole containing molecules of the invention present one or two reduction potentials.

For instance, viologens molecules usually present two reduction potentials from a bication (bipm²⁺) to the monocation (bipm⁺) and from the monocation to the neutral species) (bipm⁰).

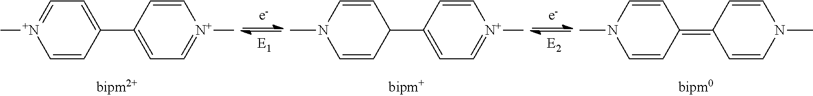

The reaction from bipm²⁺ to bipm⁺ occurs at a first potential E₁ and is a reversible reaction, while the reaction from bipm⁺ to bipm⁰ occurs at a lower potential E₂ and is often irreversible. Bipm⁰ is consider as an instable compound which can react with the oxygen present in the device or with another molecule leading to a different chemical structure and else losing its electrochromic properties.

Here the inventors have tried by modifying the chemical structures to increase the distance between the two reduction peaks in order to improve the stability of the device and hence its lifetime. Chemical groups added are such that the distance in potential between these two reduction peaks is below 0.1V, preferably below 0.3V, even preferably below 0.5V.

After conducting extensive research, the present inventors provide novel electrochromic compounds that exhibit excellent electrochromic properties and that can be easily incorporated in a cell to form, for instance, an electrochromic lens.

As such, the compounds of the present invention are advantageously:
colourless in their inactivated state and coloured, for example green, red, purple, blue, yellow or brown, in their activated state;
reversibly oxidized or reduced;
easily activated, i.e. they have an electrochemical potential from −1.5 to −0.5 V.—stable, i.e. no generation of degradation products.

More particularly the compounds of the present invention exhibit either one low reversible reduction peak or two reversible reduction peaks separated by at least 0.1V, preferably at least 0.3 V, more preferably at least 0.4 V, even more preferably at least 0.5 V the first reversible reduction peak being low).

The present invention thus relates to electrochromic compounds of formula (I) as defined below.

The present invention also relates to an electrochromic composition comprising at least one compound of formula (I).

Finally, the present invention relates to an electrochromic device, such as an ophthalmic lens, comprising an electrochromic compound of formula (I) or an electrochromic composition according to the invention.

### Definitions

The term “aromatic compound” means unsaturated chemical compounds characterized by one or more planar rings of atoms joined by covalent bonds.

The term “cyclic compound or ring compound” corresponds to a compound in which one or more series of atoms in the compound is connected to form a ring. Rings may vary in size from three to many atoms, for example 5 or 6 atoms and include examples where all the atoms are carbon (i.e., are carbocycles), or where both carbon and non-carbon atoms are present (heterocyclic compounds). More precisely, a “heterocyclic compound or ring structure” is a cyclic compound that has atoms of at least two different elements as members of its ring(s)

The term “azole” represents any five-membered heterocyclic radical containing a nitrogen atom and at least one other non-carbon atom as part of the ring. Examples of other non-carbon atoms include nitrogen, oxygen, sulfur and selenium. The five-membered heterocyclic ring (Cycle C5) in the present invention is typically an azole. Examples of azole groups include imidazole, pyrazole, oxazole, isoxazole, thiazole, isothiazole and selenazole.

The term “a conjugated system” refers to a system of connected p orbitals with delocalized electrons in a molecule, which in general lowers the overall energy of the molecule and increases stability. It is conventionally represented as having alternating single and multiple bonds. Lone pairs, radicals or carbenium ions may be part of the system, which may be cyclic, acyclic, linear or mixed. The conjugated system according to the present invention formed by the five-membered heterocyclic ring (Cycle C5) and the six-membered (hetero)cyclic ring (Cycle C6) is for example a benzazole (or benzoxazole), a benzisoxazole, a benzothiazole, a benzimidazole, an indazole, an imidazo[1,2-a]pyridine.

The term “pyridinium” refers to the cation of pyridine in which the nitrogen atom is positively charged and is represented by the following formula:

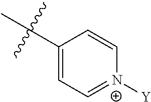

wherein Y include C₁-C₁₈ alkyl, (hetero)aryl or (hetero)arylalkyl.

The expression “alkyl” or “C₁-C₁₈ alkyl” represents any monovalent radical of a linear or branched hydrocarbon chain comprising 1 to 18 carbon atoms. The expression “C₃-C₆ alkyl” represents an alkyl group having 3 to 6 carbon atoms. Examples of C₁-C₁₈ alkyl groups include C₁-C₄ alkyl groups such as methyl, ethyl, n-propyl, i-propyl, n-butyl, i-butyl, s-butyl or t-butyl, C₆-C₈ alkyl groups such as n-hexyl, n-heptyl or n-octyl, as well as n-pentyl, 2-ethylhexyl, 3,5,5-trimethylhexyl, n-nonyl, n-decyl, n-undecyl, n-dodecyl or n-octadecyl.

The expression “aryl” represents any monovalent radical of an aromatic hydrocarbon comprising 6 to 18 carbon atoms, either monocyclic or polycyclic. The expression “Polycyclic aryls or polycyclic aromatic compounds” refers to either compounds in which at least one carbon-carbon bond is common to two aromatic rings (fused aromatic rings) or polycyclic aromatic hydrocarbons which contain two or more benzenoid rings joined by a carbon-carbon single bond. Examples of C₆-C₁₈ aryl groups include phenyl, naphthyl, anthracenyl and phenanthrenyl.

The expression “arylalkyl” represents any aryl derivative of an alkyl group. The expression “arylalkyl” represents an aryl group as defined above combined to an alkyl group as defined above. Examples of arylalkyl groups include benzyl, phenylethyl, phenylpropyl, phenylbutyl, phenylpentyl, phenylhexyl, naphthylmethyl, naphthylethyl, naphthylpropyl, naphthylbutyl, naphthylpentyl, naphthylhexyl, anthracenylmethyl, anthracenylethyl, anthracenylpropyl, anthracenylbutyl, anthracenylpentyl, anthracenylhexyl, phenanthrenylmethyl, phenanthrenylethyl, phenanthrenylpropyl, phenanthrenylbutyl, phenanthrenylpentyl and phenanthrenylhexyl.

The expression “heteroaryl” represents any monovalent radical of a monocyclic or polycyclic 5 to 10 membered aromatic group comprising from 1 to 3 heteroatoms independently selected from oxygen, nitrogen and sulfur. Examples of C₅-C₁₀ heteroaryl groups include furyl, thienyl, pyrrolyl, pyrazoyl, imidazolyl, isoxazolyl, isothiazoyl, thiazolyl, oxazolyl, 1,2,3-triazolyl, 1,2,4-triazolyl, 1-benzofuryl, 1-benzothienyl, indolyl, benzimidazolyl, indazolyl, 1,2-benzisoxazolyl, 2,1-benzisoxazolyl, 1,2-benzisothiazolyl, 2,1-benzisothiazolyl, benzothiazolyl, benzoxazolyl, benzotriazolyl, pyridyl, pyridinium, quinolinyl, quinolinium, isoquinolinyl, isoquinolinium, pyridazinyl, cinnolinyl, phthalazinyl, pyrimidinyl, quinazolinyl, pyrazinyl and quinoxalinyl.

Unless mentioned otherwise, the groups and radicals defined hereinabove may be unsubstituted or substituted by one or more substituents such as, for example, halogen, alkyl, alkoxy, aryl, heteroaryl, haloalkyl, haloalkoxy, alkoxycarbonyl, alkanoyl, aroyl, formyl, nitrile, nitro, amido, alkylthio, alkylsulfinyl, alkylsulfonyl, arylthio, arylsulfinyl, arylsulfonyl, amino, alkylamino, arylamino, dialkylamino and diarylamino.

These substituents are not a phosphonate group (P═O(OR)(OR′) with R and R′ either alkyl or aryl), a phosphate group, a carboxyl group (C═O(OH)), a trihalosilyl group such as trichlorosilyl group, a trialkoxysilyl group (such as a triethoxysilyl group or a trimethoxysilyl group), a monohalosilyl group (such as monochlorosilyl group) or a monoalkoxysilyl group.

Electrochromic Compounds

The electrochromic compounds of the present invention have a central core comprising a five-membered heterocyclic ring, typically an azole ring, fused to a six-membered (hetero)cyclic ring, onto which are branched one or several lateral pyridinium groups, in particular one, two or three lateral pyridinium groups.

As such, the electrochromic compounds of the present invention are represented by formula (I):

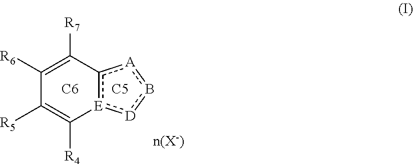

wherein:
A is N, ⁺N, N—R₁, ⁺N—R₁ or C—R₁;
B is C—R₂, S, Se, O, N, N—R₂ or ⁺N—R₂;
D is C—R₃, N, S, O, Se, N—R₃ or ⁺N—R₃;
E is C, N or ⁺N;
R₁ is H, C₁-C₁₈ alkyl, aryl or Z;
R₂ is H, C₁-C₁₈ alkyl, aryl, Z or aryl substituted by Z;
R₃ is H or C₁-C₁₈ alkyl, aryl or Z;
R₄ is H, C₁-C₁₈ alkyl, aryl or Z;
R₅ is H, C₁-C₁₈ alkyl, aryl or Z;
R₆ is H, C₁-C₁₈ alkyl, aryl or Z;
R₇ is H, C₁-C₁₈ alkyl, aryl or Z;
R₇ and R₆ and/or R₆ and R₅ and/or R₅ and R₄ may form together an aromatic ring or heteroaromatic ring fused to the six-membered (hetero)cyclic core (Cycle C6) they are attached to, optionally substituted by Z,
With Z is

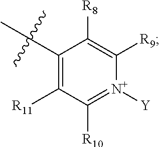

Y is C₁-C₁₈ alkyl, (hetero)aryl or (hetero)arylalkyl;
R₈, R₉, R₁₀ and R₁₁ are independently selected from H and C₁-C₁₈ alkyl;
R₈ and R₉ or R₁₀ and R₁₁ may form an aromatic ring fused to the pyridium group they are attached to,

When B=C—Z, and when A=⁺N, R₈ or R₁₁ may form with A a ring, aromatic or not, fused with the five-membered heterocyclic ring (Cycle C5) A is attached to,
n is selected to counterbalance the number of positive charges;
X is a counterion;
[symbol] is a single bond or a double bond;
   with the 3 following provisos:
1) Cycle C5 is a five membered heterocyclic ring with 2 of A, B, D and E being independently selected from: N, N—R₁, ⁺N—R₁, N—R₂, ⁺N—R₂, N—R₃, ⁺N—R₃, S, Se and O;
2) Cycle C5 and Cycle C6 form a conjugated system; and
3) at least one of R₁, R₂, R₃, R₄, R₅, R₆ or R₇ is Z or at least R₇ and R₆ form together an aromatic ring substituted by Z or at least R₅ and R₆ form together an aromatic ring substituted by Z or at least R₅ and R₄ form together an aromatic ring substituted by Z.

In all the present invention, unless otherwise stated, Y is advantageously C₁-C₆ alkyl or aryl; for example, Y is methyl, n-hexyl or phenyl.

In all the present invention, the counterion X may be selected from halide, preferably fluoride and chloride, tetrafluoroborate, tetraphenylborate, hexafluorophosphate, nitrate, methanesulfonate, trifluoromethanesulfonate, p-toluenesulfonate, hexachloroantimonate, bis(trifluoromethanesulfonyl)imide, perchlorate, acetate and sulfate, preferably, X⁻ is tetrafluoroborate or hexafluorophosphate.

The electrochromic compounds of the present invention are typically represented by formulae (II), (III), (IV), (V), (VI), (VII), (VIII), (IX) or (X):

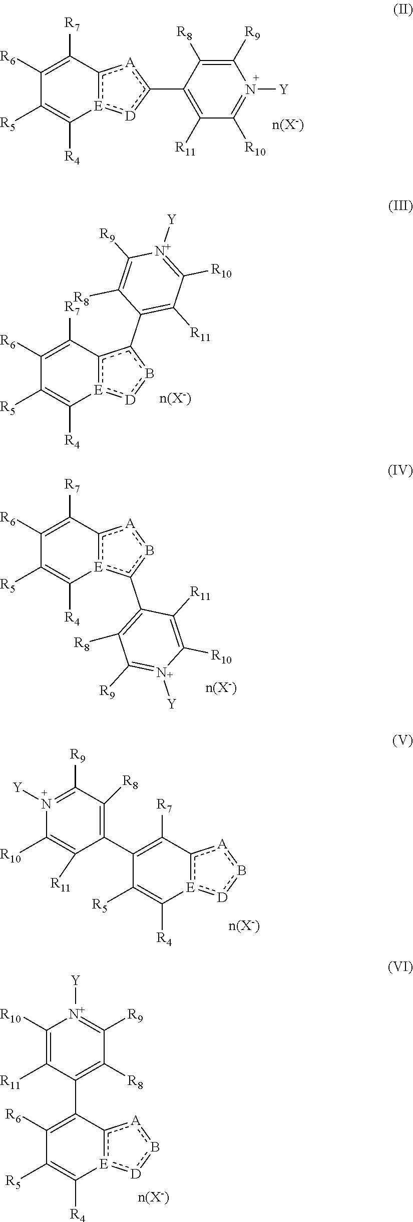

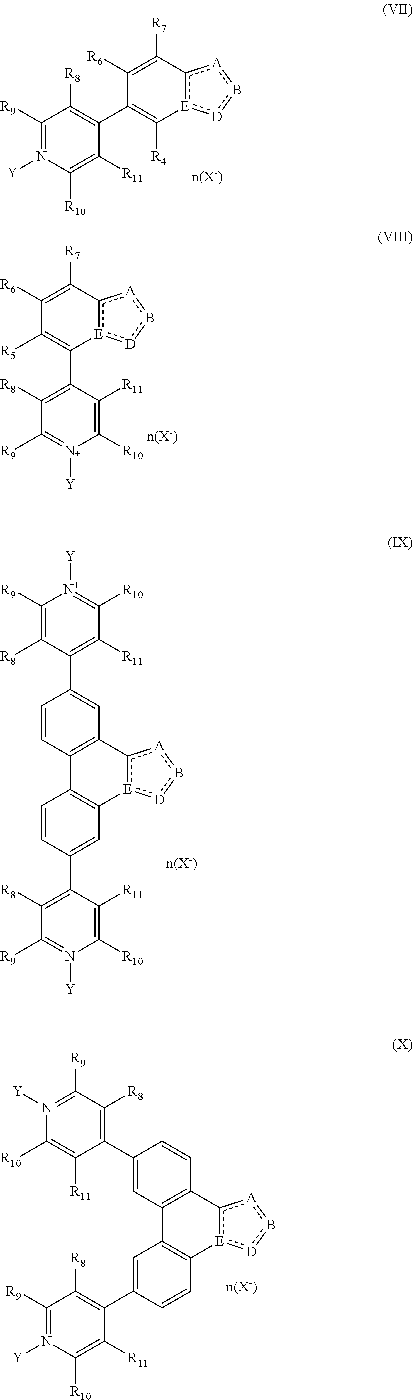

wherein A, B, D, E, R₁, R₂, R₃, R₄, R₅, R₆, R₇, R₈, R₉, R₁₀ and R₁₁, when present, and Z, Y, n and X are as described above.

In a preferred embodiment of the present invention, the five-membered heterocyclic ring C5 of the central core of the electrochromic compounds is an azole ring and the compounds of formulae (I), (II), (III), (IV), (V), (VI), (VII), (VIII), (IX) or (X) are those wherein:
A is N, ⁺N, N—R₁ or ⁺N—R₁;
and/or B is N, N—R₂ or ⁺N—R₂;
and/or D is N, N—R₃ or ⁺N—R₃.

The preferred compounds of formulae (I), (II), (III), (IV), (V), (VI), (VII), (VIII), (IX) or (X) can also be those wherein E is N or ⁺N and/or wherein D is S.

The compounds of formula (I) can also be for example:
those wherein A is ⁺N—R₁; B is C—R₂; D is S or N—R₃; E is C and R₁, R₂, R₃, R₄, R₅, R₇, R₈, R₉, R₁₀, R₁₁, Z, Y, n and X are as defined above;
those wherein A is ⁺N—R₁; B is C—R₂; D is C—R₃; E is N and R₁, R₂, R₃, R₄, R₅, R₇, R₈, R₉, R₁₀, R₁₁, Z, Y, n and X are as defined above;
those wherein A is N; B is C—R₂; D is Se or O; E is C and R₁, R₂, R₃, R₄, R₅, R₇, R₈, R₉, R₁₀, R₁₁, Z, Y, n and X are as defined above;
those wherein A is C—R₁; B is N or ⁺N—R₂; D is S; E is C and R₁, R₂, R₃, R₄, R₅, R₇, R₈, R₉, R₁₀, R₁₁, Z, Y, n and X are as defined above;
those wherein A is C—R₁; B is N, D is O; E is C and R₁, R₂, R₃, R₄, R₅, R₇, R₈, R₉, R₁₀, R₁₁, Z, Y, n and X are as defined above;
those wherein A is C—R₁; D is N, ⁺N—R₃, S, Se or O; E is C; and R₁, R₂, R₃, R₄, R₅, R₇, R₈, R₉, R₁₀, R₁₁, Z, Y, n and X are as defined above;
those wherein B is N or ⁺N—R₂; A is C—R₁; D is C—R₃; E is N; and R₁, R₂, R₃, R₄, R₅, R₇, R₈, R₉, R₁₀, R₁₁, Z, Y, n and X are as defined above;
those wherein D is ⁺N—R₃ or N; A is C—R₁; B is C—R₂; E is N; and R₁, R₂, R₃, R₄, R₅, R₇, R₈, R₉, R₁₀, R₁₁, Z, Y, n and X are as defined above; or
those wherein E is ⁺N; A is O, S, or Se; B is C—R₂; D is C—R₃. And R₁, R₂, R₃, R₄, R₅, R₇, R₈, R₉, R₁₀, R₁₁, Z, Y, n and X are as defined above.

In one variant of the present invention, the compounds of the invention are those of formula (II) wherein A is N, ⁺N, N—R₁ or ⁺N—R₁; D is N—R₃, ⁺N—R₃, S, Se, or O; E is C; and R₁, R₃, R₄, R₅, R₆, R₇, R₈, R₉, R₁₀, R₁₁, Z, Y, n and X are as defined above.

In one embodiment of this particular variant, the compounds of formula (II) are those wherein A is N⁺ or ⁺N—R₁; D is S; and E is C and R₁, R₃, R₄, R₅, R₆, R₇, R₈, R₉, R₁₀, R₁₁, Z, Y, n and X are as defined above.

According to this particular embodiment, compounds can be those of formula (II) wherein: A is ⁺N—R₁; D is S; E is C; R₁ is C₁-C₁₈ alkyl; preferably R₁ is C₁-C₆ alkyl; more preferably R₁ is methyl; R₄ is preferably H, C₁-C₁₈ alkyl or aryl; more preferably R₄ is H; R₅ is preferably H, C₁-C₁₈ alkyl or aryl; more preferably R₅ is H; R₆ is preferably H, C₁-C₁₈ alkyl or aryl; more preferably R₆ is H; R₇ is preferably H, C₁-C₁₈ alkyl or aryl; more preferably R₇ is H; R₈ is preferably H, C₁-C₁₈ alkyl or aryl; more preferably R₈ is H; R₉ is preferably H, C₁-C₁₈ alkyl or aryl; more preferably R₉ is H; R₁₀ is preferably H, C₁-C₁₈ alkyl or aryl; more preferably R₁₀ is H; R₁₁ is preferably H, C₁-C₁₈ alkyl or aryl; more preferably R₁₁ is H; Y is preferably C₁-C₁₈ alkyl or aryl; more preferably Y is C₁-C₆ alkyl or aryl; even more preferably Y is n-hexyl or phenyl; n is 2; and X⁻ is tetrafluoroborate or hexafluorophosphate, preferably tetrafluoroborate.

Other preferred compounds according to this particular embodiment are typically those of formula (II) wherein A is ⁺N; D is S; E is C and; R₈ forms with A, a five or six-membered ring, unsaturated or not, fused with Cycle C5 A is attached to and is represented by formula (XI), (XII) or (XIII).

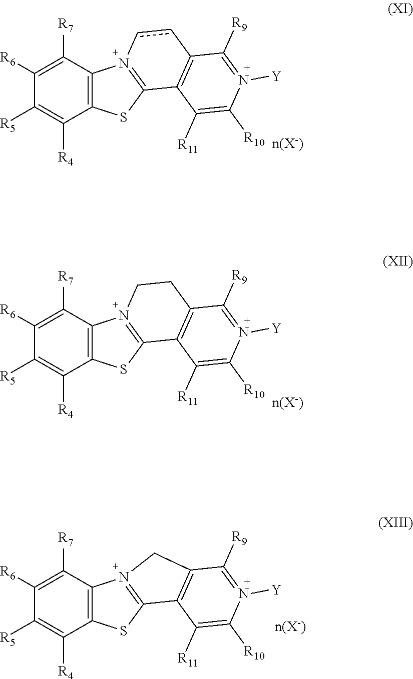

R₄, R₅, R₆, R₇, R₉, R₁₀, R₁₁, Z, Y, n and X being as defined above.

Still according to this embodiment, preferred compounds can be those of formula (II) wherein A is ⁺N—R₁; D is S; E is C; and R₁, R₄, R₅, R₆, R₇, R₈, R₉, R₁₀, R₁₁, Z, Y, n and X are as defined above. More specifically, these compounds can be the ones of Formula (II) wherein R₇ and R₆ and/or R₆ and R₅ and/or R₅ and R₄ form together an aromatic ring or heteroaromatic ring fused to the (hetero)cyclic core (Cycle C6) they are attached to, optionally substituted by Z, R₁, R₈, R₉, R₁₀, R₁₁, Z, Y, n and X being as defined above. Specific examples are represented below:

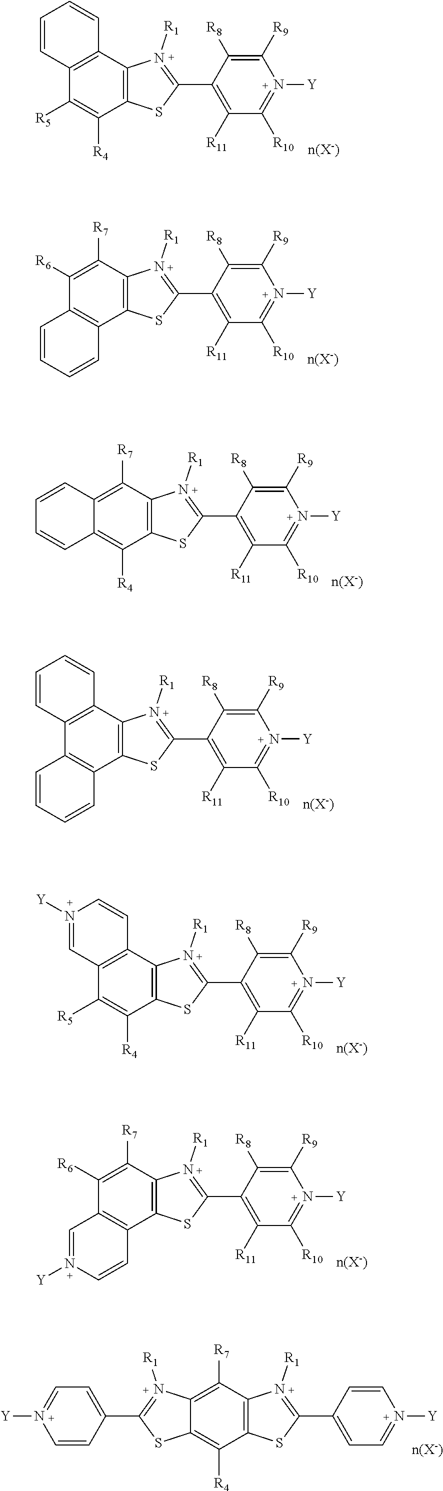

The compounds according to this embodiment can also be the ones wherein R₈ and R₉ or R₁₀ and R₁₁ form an aromatic ring fused to the pyridium group they are attached to and R₁, R₄, R₅, R₆, R₇, Z, Y, n and X are as defined above. An example of such a compound is the one shown below:

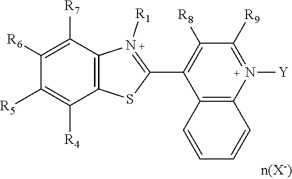

In this particular embodiment, R₁ is preferably C₁-C₁₈ alkyl; more preferably R₁ is C₁-C₆alkyl such as methyl; R₄ is preferably H, C₁-C₁₈ alkyl or aryl; more preferably R₄ is H; R₅ is preferably H, C₁-C₁₈ alkyl or aryl; more preferably R₅ is H; R₆ is preferably H, C₁-C₁₈ alkyl or aryl; more preferably R₆ is H; R₇ is preferably H; C₁-C₁₈ alkyl or aryl; more preferably R₇ is H; R₈ is preferably H, C₁-C₁₈ alkyl or aryl; more preferably R₈ is H; R₉ is preferably H, C₁-C₁₈ alkyl or aryl; more preferably R₉ is H; Y is preferably C₁-C₁₈ alkyl or aryl; more preferably Y is C₁-C₆ alkyl or aryl; even more preferably Y is phenyl; n is 2; and X⁻ is tetrafluoroborate or hexafluorophosphate.

In a second embodiment of this particular variant, the compounds of Formula (II) are those wherein A is ⁺N—R₁; D is N—R₃; E is C; and R₁, R₃, R₄, R₅, R₆, R₇, R₈, R₉, R₁₀, R₁₁, Z, Y, n and X are as defined above.

According to this particular embodiment, compounds can be those of formula (II) wherein A is ⁺N—R₁; D is N—R₃; E is C; and R₁ is C₁-C₁₈ alkyl; preferably R₁ is C₁-C₆ alkyl such as methyl or n-hexyl; R₄ is H, C₁-C₁₈ alkyl or aryl; preferably R₄ is H; R₅ is H, C₁-C₁₈ alkyl or aryl; preferably R₅ is H; R₆ is H, C₁-C₁₈ alkyl or aryl; preferably R₆ is H; R₇ is H; C₁-C₁₈ alkyl or aryl; preferably R₇ is H; R₈ is H, C₁-C₁₈ alkyl or aryl; preferably R₈ is H; R₉ is H, C₁-C₁₈ alkyl or aryl; preferably R₉ is H; R₁₀ is H, C₁-C₁₈ alkyl or aryl; preferably R₁₀ is H; R₁₁ is H, C₁-C₁₈ alkyl or aryl; preferably R₁₁ is H; Y is preferably C₁-C₁₈ alkyl or aryl; more preferably Y is C₁-C₆ alkyl or aryl; even more preferably Y is methyl, n-hexyl or phenyl; n is 2; and X⁻ is tetrafluoroborate or hexafluorophosphate, preferably hexafluorophosphate.

Other preferred compounds according to this particular embodiment are typically those of formula (II) wherein A is ⁺N—R₁; D is N—R₃; E is C; R₇ and R₆ and/or R₆ and R₅ and/or R₅ and R₄ form together an aromatic ring or heteroaromatic ring fused to the (hetero)cyclic core (Cycle C6) they are attached to, optionally substituted by Z, R₁, R₃, R₈, R₉, R₁₀, R₁₁, Z, Y, n and X being as defined above. An example of such a compound is the one shown below:

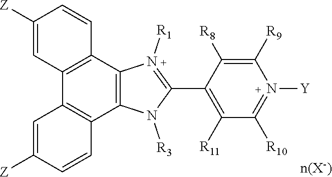

In this particular embodiment, R₁ is preferably C₁-C₁₈ alkyl; more preferably R₁ is C₁-C₆ alkyl such as methyl; R₃ is preferably C₁-C₁₈ alkyl or optionally substituted aryl, more preferably R₃ is optionally substituted aryl such as 4-t-butylphenyl; R₈ is preferably H, C₁-C₁₈ alkyl or aryl; more preferably R₈ is H; R₉ is preferably H, C₁-C₁₈ alkyl or aryl; more preferably R₉ is H; Y is preferably C₁-C₁₈ alkyl or aryl; more preferably Y is C₁-C₆ alkyl or aryl; even more preferably Y is methyl; n is 4; and X⁻ is tetrafluoroborate or hexafluorophosphate.

In a third embodiment of this particular variant, the compounds of Formula (II) are those wherein A is N; D is Se; E is C; and R₄, R₅, R₆, R₇, R₈, R₉, R₁₀, R₁₁, Z, Y, n and X are as defined above.

According to this third embodiment, compounds can be those of formula (II) wherein: A is N; D is Se; E is C; R₄ is preferably H, C₁-C₁₈ alkyl or aryl; more preferably R₄ is H; R₅ is preferably H, C₁-C₁₈ alkyl or aryl; more preferably R₅ is H; R₆ is preferably H, C₁-C₁₈ alkyl or aryl; more preferably R₆ is H; R₇ is preferably H, C₁-C₁₈ alkyl or aryl; more preferably R₇ is H; R₈ is preferably H, C₁-C₁₈ alkyl or aryl; more preferably R₈ is H; R₉ is preferably H, C₁-C₁₈ alkyl or aryl; more preferably R₉ is H; R₁₀ is preferably H, C₁-C₁₈ alkyl or aryl; more preferably R₁₀ is H; Rn is preferably H, C₁-C₁₈ alkyl or aryl; more preferably R₁₁ is H; Y is preferably C₁-C₁₈ alkyl or aryl; more preferably Y is C₁-C₆ alkyl or aryl; even more preferably Y is n-hexyl or phenyl; n is 2; and X⁻ is tetrafluoroborate or hexafluorophosphate.

In a fourth embodiment of this particular variant, the compounds of Formula (II) are those wherein A is N; D is O; E is C; and R₄, R₅, R₆, R₇, R₈, R₉, R₁₀, R₁₁, Z, Y, n and X are as defined above.

According to this fourth embodiment, compounds can be those of formula (II) wherein: A is N; D is O; E is C; R₄ is preferably H, C₁-C₁₈ alkyl or aryl; more preferably R₄ is H; R₅ is preferably H, C₁-C₁₈ alkyl or aryl; more preferably R₅ is H; R₆ is preferably H, C₁-C₁₈ alkyl or aryl; more preferably R₆ is H; R₇ is preferably H, C₁-C₁₈ alkyl or aryl; more preferably R₇ is H; R₈ is preferably H, C₁-C₁₈ alkyl or aryl; more preferably R₈ is H; R₉ is preferably H, C₁-C₁₈ alkyl or aryl; more preferably R₉ is H; R₁₀ is preferably H, C₁-C₁₈ alkyl or aryl; more preferably R₁₀ is H; Rn is preferably H, C₁-C₁₈ alkyl or aryl; more preferably Rn is H; Y is preferably C₁-C₁₈ alkyl or aryl; more preferably Y is C₁-C₆ alkyl or aryl; even more preferably Y is n-hexyl or phenyl; n is 2; and X⁻ is tetrafluoroborate or hexafluorophosphate.

In a second variant of the present invention, the compounds of the invention are those of Formula (II) wherein A is ⁺N—R₁; D is C—R₃; E is N; and R₁, R₄, R₅, R₆, R₇, R₈, R₉, R₁₀, R₁₁, Z, Y, n and X are as defined above.

According to this variant, the compounds of the invention can be those wherein R₁ is preferably C₁-C₁₈ alkyl; more preferably R₁ is C₁-C₆ alkyl such as methyl; R₃ is preferably H or C₁-C₁₈ alkyl, more preferably R₃ is H; R₄ is preferably H, C₁-C₁₈ alkyl or aryl; more preferably R₄ is H; R₅ is preferably H, C₁-C₁₈ alkyl or aryl; more preferably R₅ is H; R₆ is preferably C₁-C₁₈ alkyl, aryl or Z; more preferably R₆ is aryl or Z; R₇ is preferably H, C₁-C₁₈ alkyl or aryl; more preferably R₇ is H; R₈ is preferably H, C₁-C₁₈ alkyl or aryl; more preferably R₈ is H; R₉ is preferably H, C₁-C₁₈ alkyl or aryl; more preferably R₉ is H; R₁₀ is preferably H, C₁-C₁₈ alkyl or aryl; more preferably R₁₀ is H; Rn is preferably H, C₁-C₁₈ alkyl or aryl; more preferably Rn is H; Y is preferably C₁-C₁₈ alkyl or aryl; more preferably Y is C₁-C₆ alkyl or aryl; even more preferably Y is n-hexyl or phenyl; n is preferably 2 or 3; and X⁻ is tetrafluoroborate or hexafluorophosphate.

In a third variant of the present invention, the compounds of the invention are those of Formula (III) wherein B is N or ⁺N—R₂; D is S; E is C; and R₂, R₄, R₅, R₆, R₇, R₈, R₉, R₁₀, R₁₁, Z, Y, n and X are as defined above.

According to this third variant of the present invention, the compounds of the invention can be the ones wherein R₂, when present, is preferably C₁-C₁₈ alkyl, more preferably R₂ is C₁-C₆ alkyl such as methyl; R₄ is preferably H, C₁-C₁₈ alkyl or aryl; more preferably R₄ is H; R₅ is preferably H, C₁-C₁₈ alkyl or aryl; more preferably R₅ is H; R₆ is preferably C₁-C₁₈ alkyl, aryl; more preferably R₆ is H; R₇ is preferably H, C₁-C₁₈ alkyl or aryl; more preferably R₇ is H; R₈ is preferably H, C₁-C₁₈ alkyl or aryl; more preferably R₈ is H; R₉ is preferably H, C₁-C₁₈ alkyl or aryl; more preferably R₉ is H; R₁₀ is preferably H, C₁-C₁₈ alkyl or aryl; more preferably R₁₀ is H; Rn is preferably H, C₁-C₁₈ alkyl or aryl; more preferably Rn is H; Y is preferably C₁-C₁₈ alkyl or aryl; more preferably Y is C₁-C₆ alkyl or aryl; even more preferably Y is n-hexyl or phenyl; n is 1 or 2; and X⁻ is tetrafluoroborate or hexafluorophosphate.

In a fourth variant of the present invention, the compounds of the invention are those of Formula (III) wherein B is N; D is O; E is C; and R₂, R₄, R₅, R₆, R₇, R₈, R₉, R₁₀, R₁₁, Z, Y, n and X are as defined above.

According to this fourth variant of the present invention, the compounds of the invention can be the ones wherein R₄ is preferably H, C₁-C₁₈ alkyl or aryl; more preferably R₄ is H; R₅ is preferably H, C₁-C₁₈ alkyl or aryl; more preferably R₅ is H; R₆ is preferably C₁-C₁₈ alkyl, aryl; more preferably R₆ is H; R₇ is preferably H, C₁-C₁₈ alkyl or aryl; more preferably R₇ is H; R₈ is preferably H, C₁-C₁₈ alkyl or aryl; more preferably R₈ is H; R₉ is preferably H, C₁-C₁₈ alkyl or aryl; more preferably R₉ is H; R₁₀ is preferably H, C₁-C₁₈ alkyl or aryl; more preferably R₁₀ is H; R₁₁ is preferably H, C₁-C₁₈ alkyl or aryl; more preferably R₁₁ is H; Y is preferably C₁-C₁₈ alkyl or aryl; more preferably Y is C₁-C₆ alkyl or aryl; even more preferably Y is n-hexyl or phenyl; n is 1; and X⁻ is tetrafluoroborate or hexafluorophosphate.

In a fifth variant of the present invention, the compounds of the invention are those of Formula (V) wherein A is ⁺N—R₁; B is C—R₂; D is C—R₃; E is N; and R₁, R₂, R₃, R₄, R₅, R₇, R₈, R₉, R₁₀, R₁₁, Z, Y, n and X are as defined above.

According to this fifth variant of the present invention, the compounds of the invention can be the ones wherein R₁ is preferably C₁-C₁₈ alkyl; more preferably R₁ is C₁-C₆ alkyl such as methyl; R₂ is preferably C₁-C₁₈ alkyl or aryl, more preferably R₂ is aryl such as phenyl; R₃ is preferably H, C₁-C₁₈ alkyl or aryl; more preferably R₃ is H; R₄ is preferably H, C₁-C₁₈ alkyl or aryl; more preferably R₄ is H; R₅ is preferably H, C₁-C₁₈ alkyl or aryl; more preferably R₅ is H; R₇ is preferably H, C₁-C₁₈ alkyl or aryl; more preferably R₇ is H; R₈ is preferably H, C₁-C₁₈ alkyl or aryl; more preferably R₈ is H; R₉ is preferably H, C₁-C₁₈ alkyl or aryl; more preferably R₉ is H; R₁₀ is preferably H, C₁-C₁₈ alkyl or aryl; more preferably R₁₀ is H; Rn is preferably H, C₁-C₁₈ alkyl or aryl; more preferably Rn is H; Y is preferably C₁-C₁₈ alkyl or aryl; more preferably Y is C₁-C₆ alkyl or aryl; even more preferably Y is n-hexyl or phenyl; n is 2; and X⁻ is tetrafluoroborate or hexafluorophosphate.

In a sixth variant of the present invention, the compounds of the invention are those of Formula (IX) or (X) wherein A is N—R₁; B is C—R₂; D is C—R₃; E is N; and R₁, R₂, R₃, R₈, R₉, R₁₀, R₁₁, Z, Y, n and X are as defined in above.

According to this sixth variant of the present invention, the compounds of the invention can be the ones wherein R₁ is preferably C₁-C₁₈ alkyl; more preferably R₁ is C₁-C₆ alkyl such as methyl; R₂ is preferably substituted aryl, more preferably R₂ is 4-t-butylphenyl or phenyl substituted by Z; R₃ is preferably C₁-C₁₈ alkyl or aryl; more preferably R₃ is aryl, for example 4-t-butylphenyl; R₈ is preferably H, C₁-C₁₈ alkyl or aryl; more preferably R₈ is H; R₉ is preferably H, C₁-C₁₈ alkyl or aryl; more preferably R₉ is H; R₁₀ is preferably H, C₁-C₁₈ alkyl or aryl; more preferably R₁₀ is H; R₁₁ is preferably H, C₁-C₁₈ alkyl or aryl; more preferably R₁₁ is H; Y is preferably C₁-C₁₈ alkyl or aryl; more preferably Y is C₁-C₆ alkyl such as methyl; n is 3 or 4; and X⁻ is tetrafluoroborate or hexafluorophosphate.

In a particularly preferred embodiment of the present invention, the compound of formula (I) is selected from the group consisting of:

| Compound | Structure |
|---|---|
| 1 | 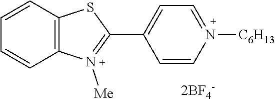 |
| 2 | 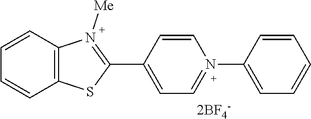 |
| 3 | 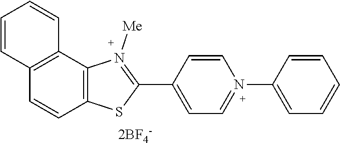 |
| 4 | 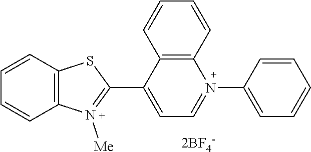 |
| 5 | 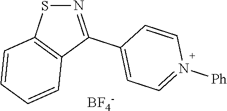 |
| 6 | 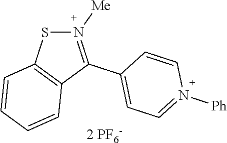 |
| 7 | 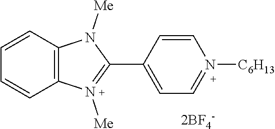 |
| 8 | 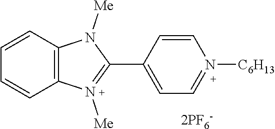 |
| 9 | 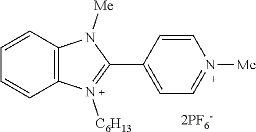 |
| 10 | 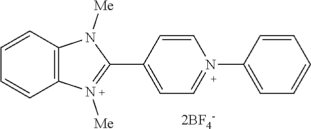 |
| 11 | 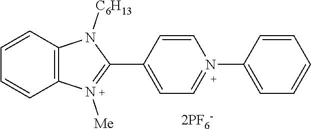 |
| 12 | 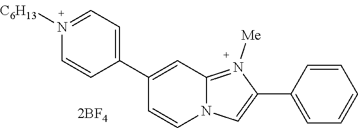 |
| 13 | 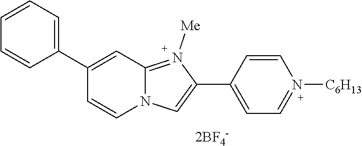 |
| 14 | 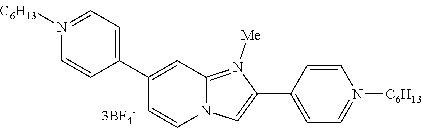 |
| 15 | 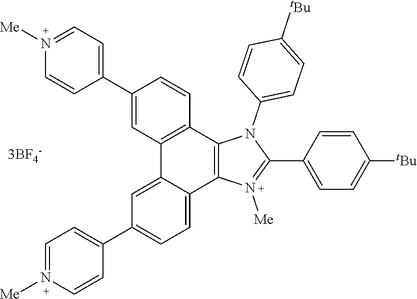 |
| 16 | 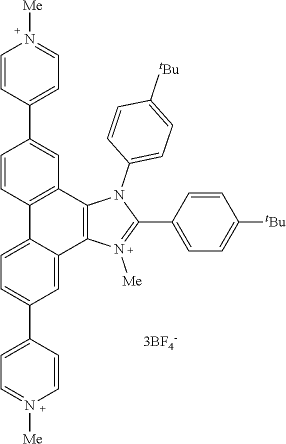 |
| 17 | 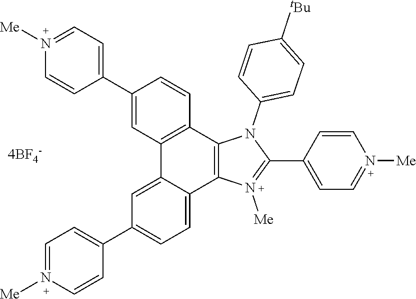 |
| 18 | 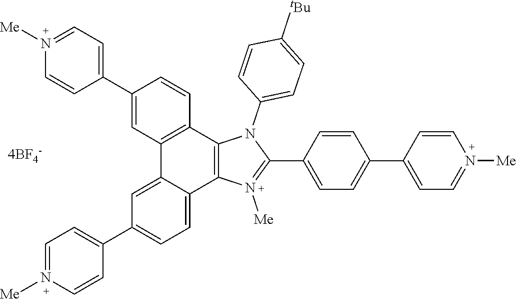 |
| 19 | 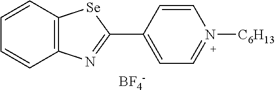 |
| 20 | 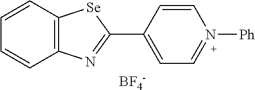 |
| 21 | 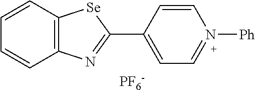 |
| 22 | 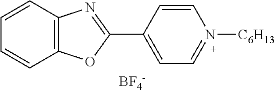 |
| 23 | 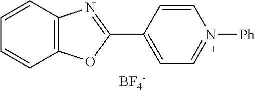 |
| 24 | 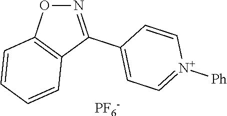 |

Compounds represented by formula (I) may be prepared according to various methods well known in the art.

For example, compounds represented by formula (I) may be obtained according to the synthetic routes detailed hereinafter.

A variety of routes to access benzimidazole and benzothiazole derivatives have been described; the simplest and most useful of which involve annulation of the heterocyclic ring onto a 1,2-phenylenediamine or a 2-aminothiophenol respectively. Synthetic routes involving these precursors have been reviewed. (J. Revuelta, F. Machetti and S. Cicchi in Modern Heterocyclic Chemistry eds. J. Alvarez-Builla, J. J. Vaquero and J. Barluenga, Wiley-WCH, Weinheim, 2011, vol. 2, pp. 809-923.)

Although 2-(hetero)arylbenzothiazoles are accessible via a tandem acylation-cyclodehydration sequence of a 2-aminothiophenol, other routes are known. For example, 2-(4-pyridyl)benzothiazole was first obtained from the high temperature reaction of 4-picoline with sulfur in the presence of aniline, or from isonicotinic acid and 2-aminothiophenol in the presence of thionyl chloride. Moreover the same compound could be obtained by cyclocondensation of 2-aminothiophenol with pyridine-4-carboxaldehyde and subsequent oxidation of the resulting benzothiazoline with iron(III) sulfate (P. E. Miller, G. L. Oliver, J. R. Dann and J. W. Gates Jr., J. Org. Chem., 1957, 22, 664). More recently it has been shown that benzothiazoles can also be obtained from aldehydes or alkanophenones and 2-aminothiophenol; the oxidation step is accomplished by aerial oxidation in DMSO (Y. Liao, H. Qi, S. Chen, P. Jiang, W. Zhou and G.-J. Deng, Org. Lett., 2012, 14, 6004). A combination of these approaches was used in the present work in which an ethanolic solution of 2-aminothiophenol and pyridine-4-carboxaldehyde was stirred in air. Subsequent N-alkyation with a haloalkane e.g. 1-iodohexane proceeded readily on the pyridine moiety as expected (K. Halman and O. H. Hankovszky, Acta Chim. Acad. Sci. Hung., 1965, 43, 263; P. Zavins, E. S. Lavinovitch and A. Arens, Khim. Geterotsikl. Soedin., 1973, 104). Thus 1-iodohexane provided a good yield of the iodide salt and ultimately the tetrafluoroborate following anion exchange. The latter could then be further alkylated on N-3 of the benzothiazole moiety with methyl tosylate as shown in scheme 1.

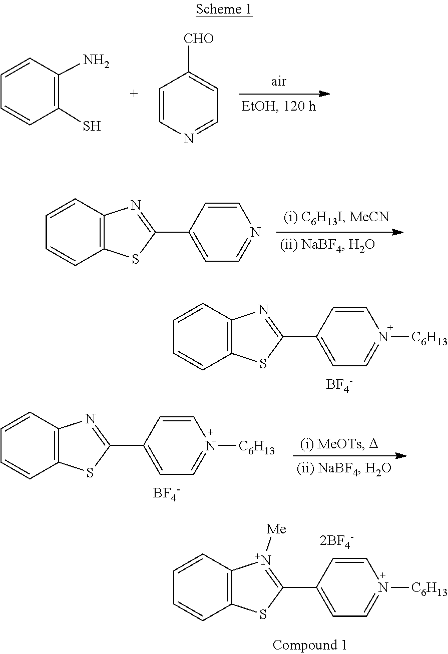

Although 2-(1-phenylpyridin-4-yl-1-ium)benzothiazole has been synthesised by a convoluted sequence using pyrylium salt chemistry (G. N. Dorofeenko, A. V. Koblik, B. A. Tertov and T. I. Polyakova, Khim. Geterotsikl. Soedin., 1973, 1016) a simpler approach to this and derived salts is illustrated in scheme 6. The key step involves N-arylation of the pyridine moiety with readily available diphenyliodonium triflate (M. Bielawski and B. Olofsson, Chem. Commun., 2007, 2521) under copper catalysis. This approach has been employed for the N-phenylation of a range of other heterocyclic systems (T. Lv, Z. Wang, J. You, J. Lan and G. Gao, J. Org. Chem., 2013, 73, 5723; C. Reus, M. Stolar, J. Vanderkley, J. Neubauer and T. Baumgartner, J. Am. Chem. Soc., 2015, 137, 11710). Alkylation of the benzothiazole with methyl tosylate proceeds straightforwardly.

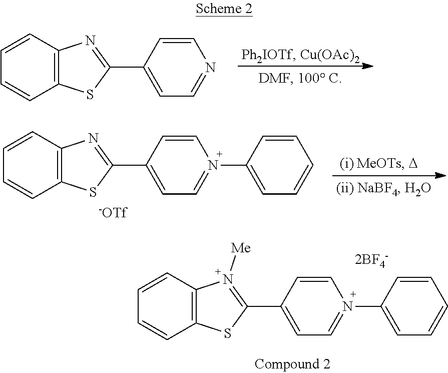

Similar sequences to those depicted in Schemes 1 and 2 are applicable to readily available quinoline-4-carbaldehyde (W.-Z. Weng, J.-S. Guo, K.-X. Liu, T.-Q. Shao, L.-Q. Song, Y.-P. Zhu, Y.-Y. Sun and Q.-G. Meng, Can. J. Chem., 2020, 98, 179) and the derived benzothiazole.

Although accessible from o-phenylenediamine (OPD) and carboxylic acid derivatives, both 2-(2-pyridyl)- and 2-(4-pyridyl)-benzimidazole were obtained efficiently via condensation-aerial oxidation of the appropriate pyridinecarboxaldehyde with OPD (cf. S. Haneda, Z. Gan, K. Eda and M. Hayashi, Organometallics, 2007, 26, 6551). 2-(4-Pyridyl)benzimidazoles could be selectively methylated in the imidazole ring by treatment with Mel under basic conditions. Sequential alkylations within the pyridine moiety and benzimidazole rings have been accomplished. In a similar manner to that employed previously, the Cu-mediated N-arylation of the pyridine moiety in 1-methyl-2-(4-pyridyl)benzimidazole with diphenyliodonium triflate followed by treatment with a haloalkane or with MeOTs has provided access to a novel series of compounds (Scheme 3).

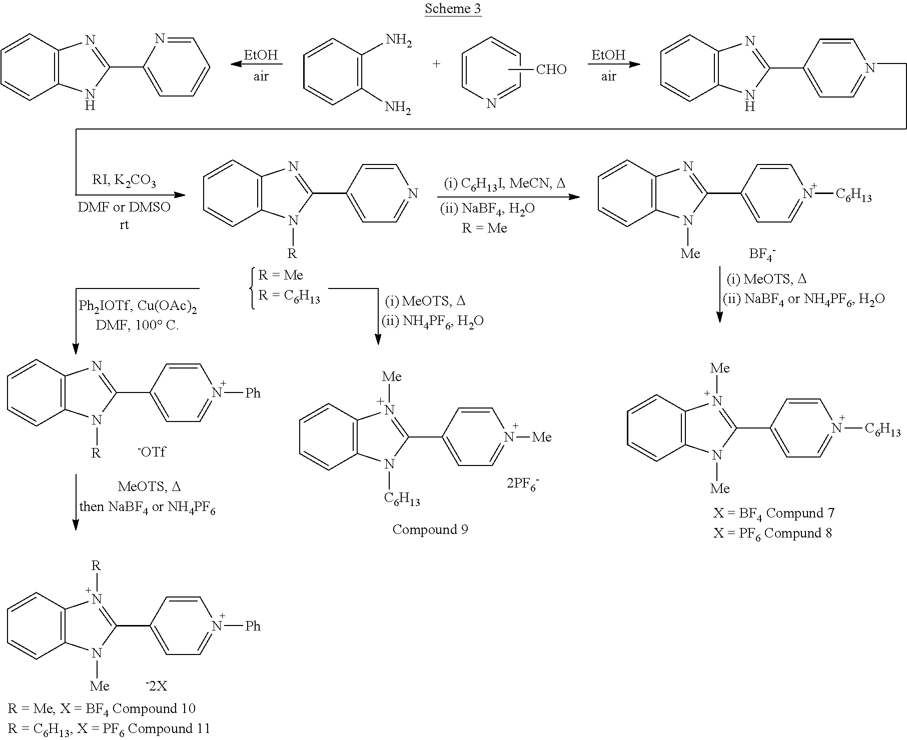

Imidazo[1,2-a]pyridines are available by a variety of routes and a number of reviews of the synthesis and chemistry of these compounds are available (H. L. Blewitt in Special Topics in Heterocyclic Chemistry eds. A. Weissberger and E. C. Taylor, Wiley-Interscience, New York, 1977, pp. 117-178; F. Couty and G. Evano, Comprehensive Heterocyclic Chemistry III eds. A. R. Katritzky, C. A. Ramsden, E. F. V. Scriven and R. J. K. Taylor, Elsevier, Oxford, 2008, vol. 11, pp. 409-499; A. K. Bagdi, S. Santra, K. Monir and A. Hajra, Chem. Commun., 2015, 51, 1555; S. M. Roopan, S. M. Patil and J. Palaniraja, Res. Chem. Intermed., 2016, 42, 2740). By far the most useful entry to these bicyclic rings involves the N-alkylation-cyclocondensation of 2-aminopyridines with α-bromo ketones. Thus, reaction of commercially available 2-amino-4-bromopyridine with phenacyl bromide in methanol in the presence of NaHCO₃ provided 7-bromo-2-phenylimidazo[1,2-a]pyridine A [K. C. L. Lee and E. T. Sun, PCT WO2006/101455A1 (2006)]. An analogous reaction in which 4-(bromoacetyl)pyridine hydrobromide (M. P. Hay, S. Turcette, J. U. Flannagan, M. Bonnet, D. A. Chan, P. D. Sutphin, P. Nguyen, A. J. Graccia and W. A. Denny, J. Med. Chem., 2010, 53, 787) replaced phenacyl bromide furnished 7-bromo-2-(4-pyridyl)imidazo[1,2-a]pyridine B in good yield. Suzuki-Miyaura coupling of A with 4-(4,4,5,5-tetramethyl-1,3,2-dioxaborolan-2-yl)pyridine provided 2-phenyl-7-(4-pyridyl)imidazo[1,2-a]pyridine C in high yield. Application of this Pd-coupling reaction to B was employed to generate the 7-phenyl derivative D and the bis-pyridyl compound E. Alkylation of C and D with 1-iodohexane in MeCN proceeded on the pyridine nitrogen to give the salts F and G respectively. Further alkylation of F and G at N−1 was accomplished by heating with methyl tosylate. Bis-alkylation of both pyridine moieties in E could be accomplished by heating with excess alkyl halide to provide H that could also be methylated on N−1 with methyl tosylate. (Scheme 4)

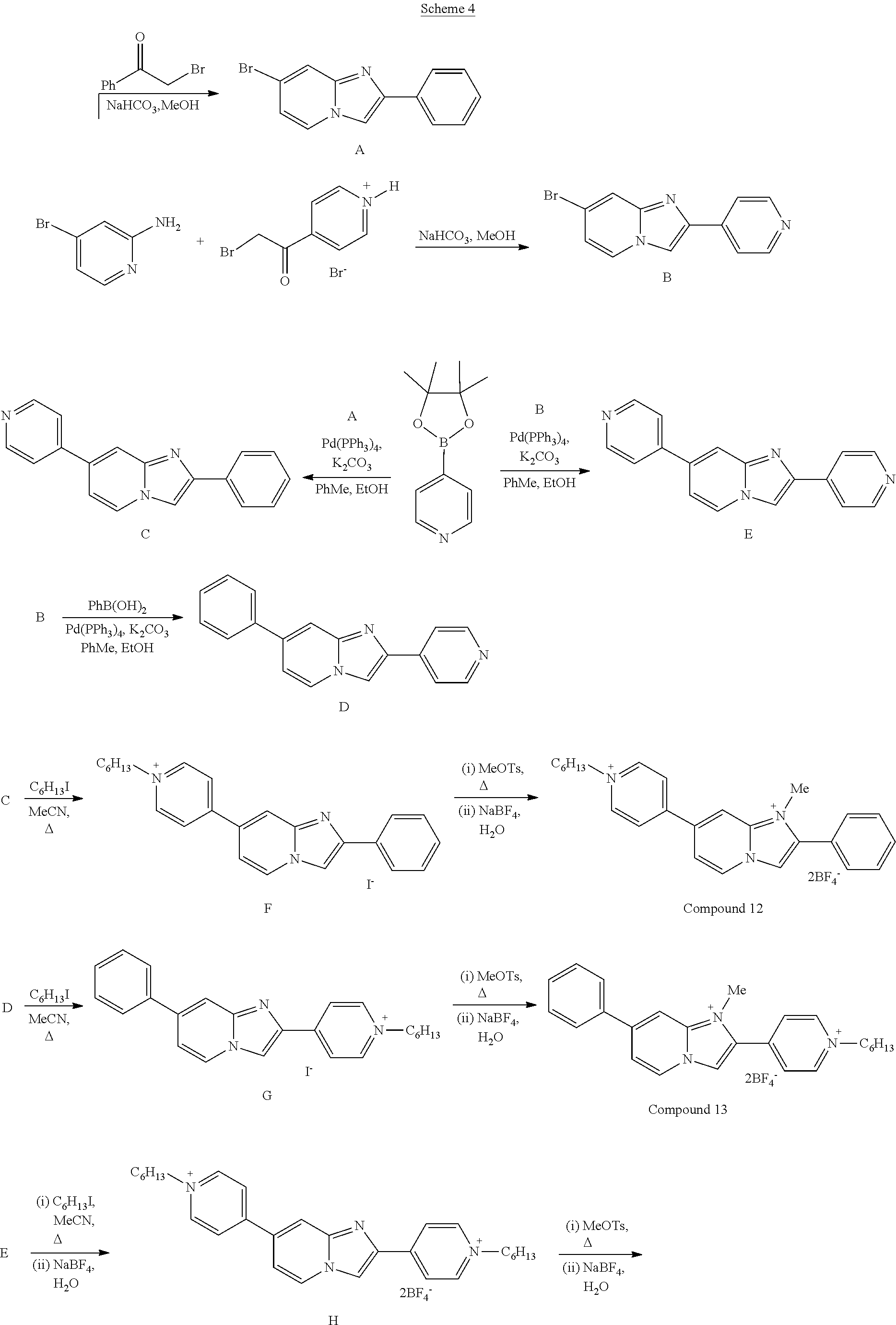

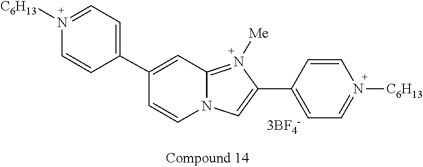

4,4′-Dibromobenzil is widely commercially available and the isomeric dibromophenanthrene-9,10-diones, 3,6-dibromophenanthrene-9,10-dione (M. O. BaniKhaled, J. D. Mottishaw, H. Sun, Cryst. Growth Des., 2015, 15, 2235; A. Patel, S. Y. Sharp, K. Hall, W. Lewis, M. F. G. Stevens, P. Workman, C. J. Moody, Org. Biomol. Chem., 2016, 14, 3889) and 2,7-dibromophenanthrene-9,10-dione (T. H. Vo, M. Shekhirev, D. A. Kunkel, F. Orange, M. J.-F. Guinel, A. Endersbe, A. Sinitskii, Chem. Commun., 2014, 50, 4172), were prepared by selective bromination of phenanthrene-9,10-dione according to published literature procedures.

The multicomponent condensation reaction between a 1,2-dicarbonyl compound an aromatic aldehyde, an aniline and a small molecule capable of providing a nitrogen atom such as ammonium acetate in a suitable medium is a versatile route to highly substituted imidazoles and fused imidazoles (K. Skonieczny, D. T. Gryko, J. Org. Chem., 2015, 80, 5753; M. M. Heravil, M. Daraiel, V. Zadsirjan, Mol. Divers., 2015, 19, 577; D. Kumar, K. R. J. Thomas, J. Photochem. Photobiol. A: Chem., 2011, 218, 162). The synthesis of 6,9-dibromo-1,2-bis(4-(tert-butyl)phenyl)-1H-phenanthro[9,10-d]imidazole has been accomplished by such a multicomponent condensation between 3,6-dibromophenanthrene-9,10-dione, 4-tert-butylbenzaldehyde, 4-tert-butylaniline and ammonium acetate in acetic acid (W-C. Chen, Y. Yuan, Y. Xiong, A. L. Rogach, Q-X. Tong, C-S. Lee, ACS Appl. Mater. Interfaces, 2017, 9, 26268). Using identical reaction conditions and reagents 2,7-dibromophenanthrene-9,10-dione was transformed into 5,10-dibromo-1,2-bis(4-(tert-butyl)phenyl)-1H-phenanthro[9,10-d]imidazole (Scheme 5).

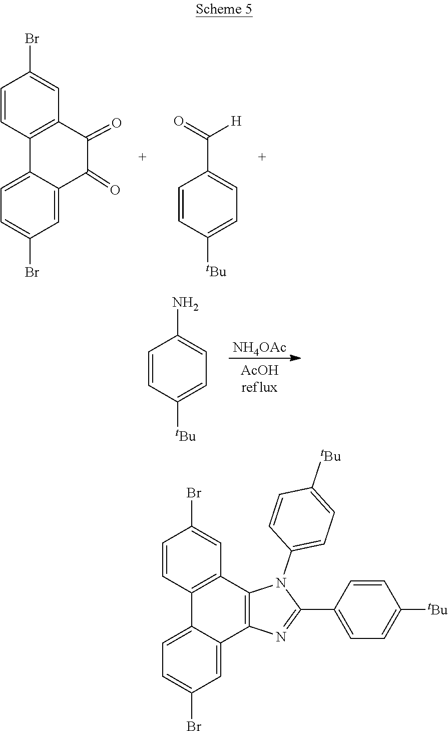

Repeating the multicomponent condensation depicted in scheme 1 but starting from 3,6-dibromophenanthrene-9,10-dione and using either 4-bromobenzaldehyde or pyridine-4-carboxaldehyde as the aldehyde component gave 6,9-dibromo-2-(4-bromophenyl)-1-(4-(tert-butyl)phenyl)-1H-phenanthro[9,10-d]imidazole or 6,9-dibromo-1-(4-(tert-butyl)phenyl)-2-(pyridin-4-yl)-1H-phenanthro[9,10-d]imidazole, respectively (Scheme 6).

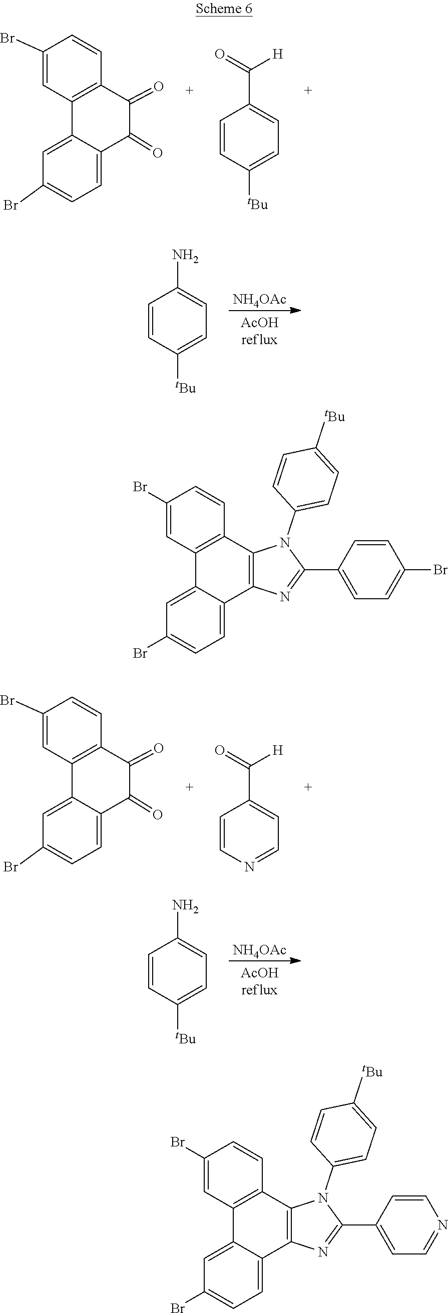

The foregoing bromo substituted imidazoles and phenanthro[9,10-d]imidazoles were each subjected to a generic Suzuki cross-coupling protocol with 4-(4,4,5,5-tetramethyl-1,3,2-dioxaborolan-2-yl)pyridine employing a carbonate base with a palladium catalyst, typically tetrakis(triphenylphosphine)palladium(0), in a mixed solvent system to afford the poly (4-pyridyl)substituted imidazoles and phenanthro[9,10-d]imidazoles (Scheme 7). The Suzuki coupling of 4-(4,4,5,5-tetramethyl-1,3,2-dioxaborolan-2-yl)pyridine with aryl bromides has been widely reported (M. Jurícek, J. C. Barnes, E. J. Dale, W-G. Liu, N. L. Strutt, C. J. Bruns, N. A. Vermeulen, K. C. Ghooray, A. A. Sarjeant, C. L. Stern, Y. Y. Botros, W. A. Goddard, J. F. Stoddart, J. Am. Chem. Soc., 2013, 135, 12736; Y. Nakamura, N. Aratani, A. Osuka, Chem. Asian J., 2007, 2, 860; V. Gray, K. Börjesson, D. Dzebo, M. Abrahamsson, B. Albinsson, K. Moth-Poulsen, J. Phys. Chem. C, 2016, 120, 19018) (Scheme 7).

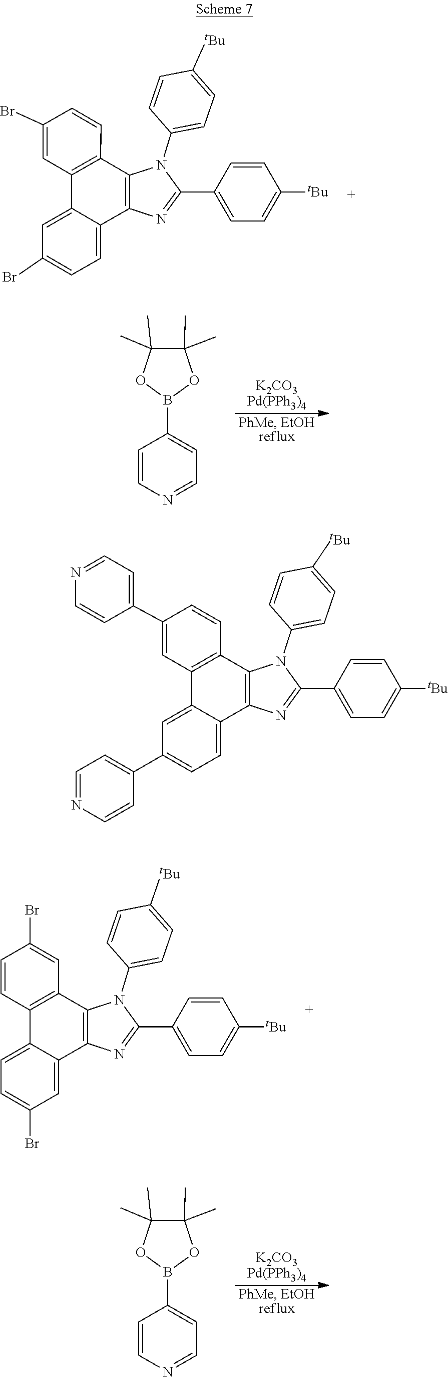

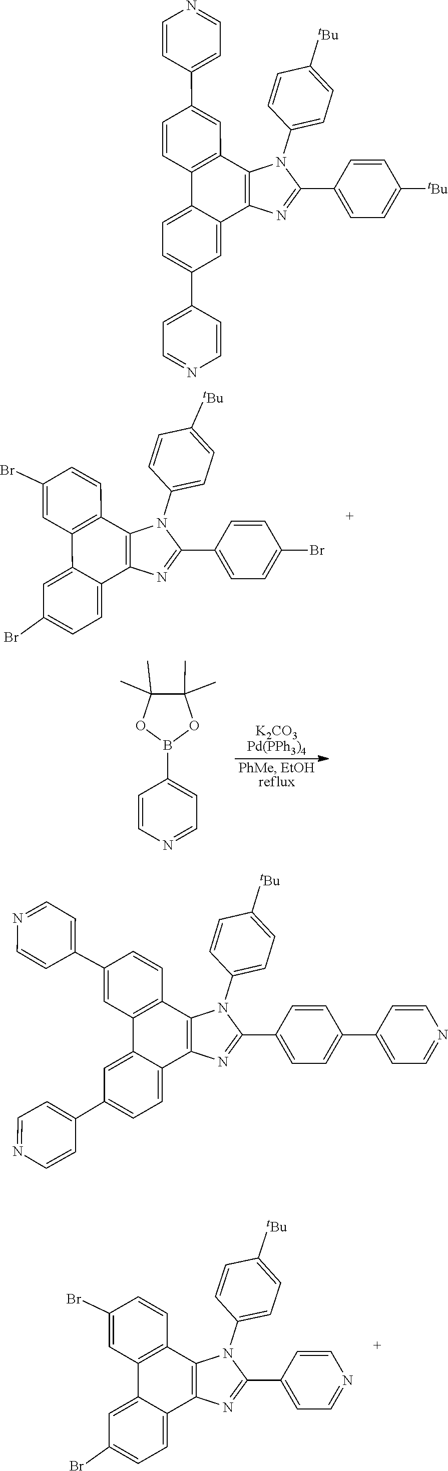

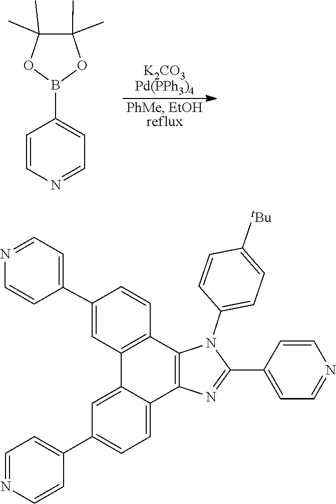

Methyl tosylate has been used as an effective methylating agent either neat or with a co-solvent (J. F. S. Carvalho, J. Louvel, M. L. J. Doornbos, E. Klaase, Z. Yu, J. Brussee, A. P. Ijzerman, J. Med. Chem., 2013, 56, 2828; A. N. Wodward, J. M. Kolesar, S. R. Hall, N-A. Saleh, D. S. Jones, M. G. Walter, J. Am. Chem. Soc., 2017, 139, 8467; L. Pescatori, A. Arduini, A. Pochini, A. Secchi, C. Massera, F. Ugozzoli, Org. Biomol. Chem., 2009, 7, 3698; M. Kuroboshi, T. Yamamoto, H. Tanaka, Synlett, 2013, 24, 0197; J. A. Zoltewicz, M. P. Cruskie, Jr., Tetrahedron, 1995, 51, 3103). In this work methyl tosylate was employed to simultaneously N-methylate the pyridine ring N-atoms and N3- of the imidazole ring. The resulting poly (tosylate) salts were directly converted into their fluoroborate salts (Scheme 8).

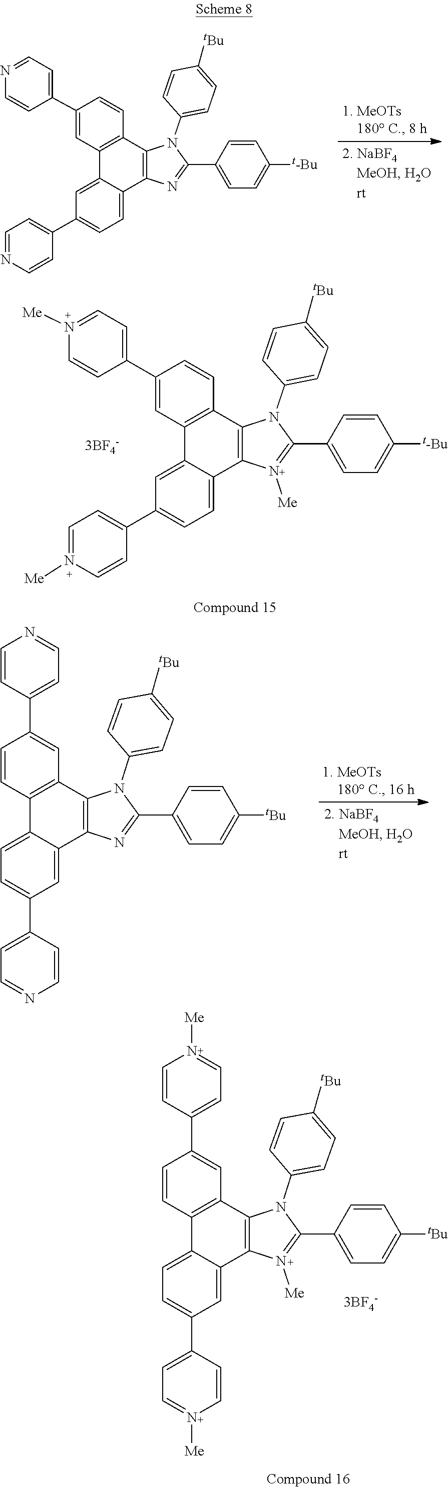

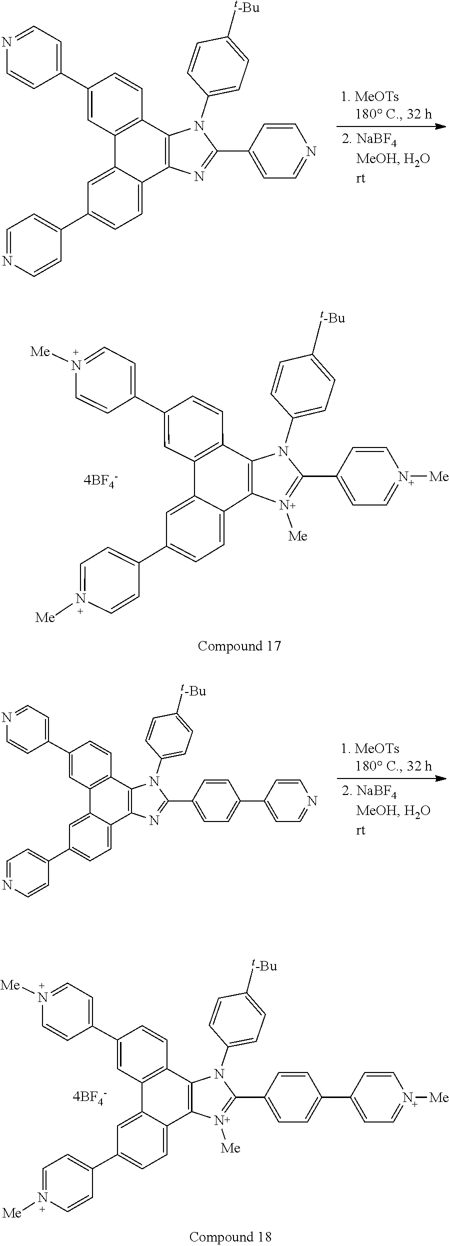

2-(Pyridin-4-yl)benzoselenazole was synthesised according to a modified literature procedure (T. Su, S. Xie, B. Li, J. Yan, L. Huang, X. Li, Synlett 2015; 26, 215). Selective alkylation of the pyridine N-atom of 2-(pyridin-4-yl)benzoselenazole was accomplished upon reaction with an appropriate alkyl halide (Scheme 11). Alternatively, pyridine N-atom arylation of 2-(pyridin-4-yl)benzoselenazole was accomplished using an aryliodonium salt according to general literature protocols (T. Lv, Z. Wang, J. You, J. Lan and G. Gao, J. Org. Chem., 2013, 73, 5723; C. Reus, M. Stolar, J. Vanderkley, J. Neubauer and T. Baumgartner, J. Am. Chem. Soc., 2015, 137, 11710). A final counterion exchange with either NaBF₄ or NH₄PF₆ afforded the target electrochromic compounds (Scheme 9).

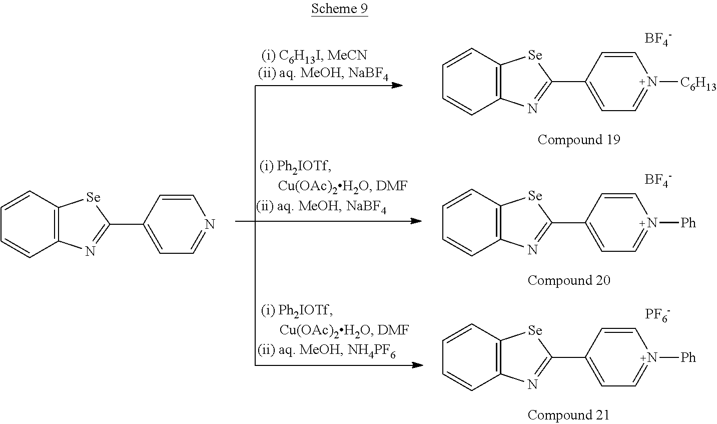

Benzoxazoles are accessible by similar routes used to access the benzothiazoles. The condensation of 2-aminophenol with pyridine-4-carboxaldehyde in the presence of air affords exclusively the 2,3-dihydrobenzoxazole derivative. Subsequent dehydrogenation to 2-(4-pyridyl)benzoxazole is readily accomplished by treatment with 2,3-dichloro-5,6-dicyano-1,4-benzoquinone (DDQ) in dichloromethane. Both N-alkylation and N-arylation of the pyridine moiety can be accomplished by standard procedures (Scheme 10).

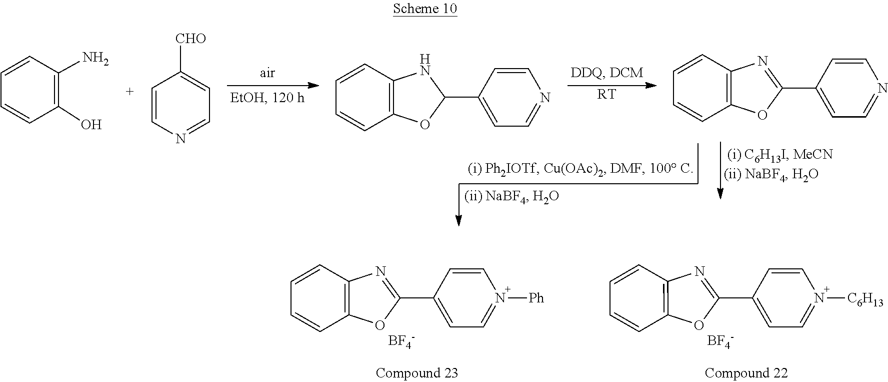

Numerous methods are available to construct other fused-ring azoles. For example, 2-arylnaphtho[1,2-d]thiazoles are accessible from a one-pot dehydrogenation (Semmler-Wolff aromatisation)-sulfur transfer-cyclocondensation sequence between 1-tetralone oxime 0-acetate and aryl aldehydes in the presence of elemental sulfur in DMSO (Z. Xu, H. Huang, H. Chen and G.-J. Deng, Org. Chem. Front., 2019, 6, 3060). In this way, 2-(pyridin-4-yl)benzothiazole has been obtained and the pyridine moiety selectively N-arylated by the usual means with diphenyliodonium triflate under Cu-catalysis. Subsequent N-methylation of the thiazole ring was accomplished by treatment with MeOTf. (Scheme 11) These quaternisation reactions are widely applicable to other bi-heterocyclic systems.

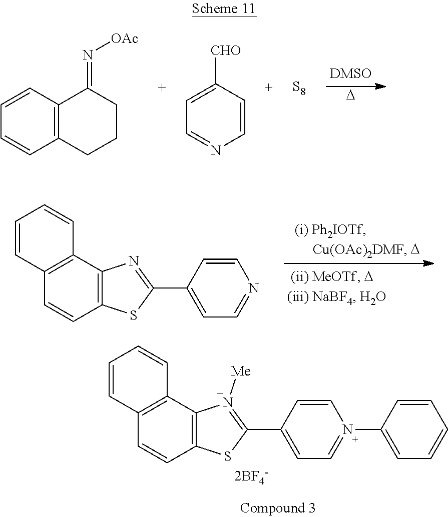

3-Aryl-1,2-benzisothiazoles including the 3-(pyridin-4-yl)- derivative can be accessed via quenching the thioanisole dianion (obtained by lithiation with BuLi-TMEDA in TBME) with 4-pyridinecarbonitrile (R. Zhu, Z. Liu, J. Chen, X. Xiong, Y. Wang, L. Huang, J. Bai, Y. Deng and J. Huang, Org. Lett., 2018, 20, 3161) (Scheme 12)

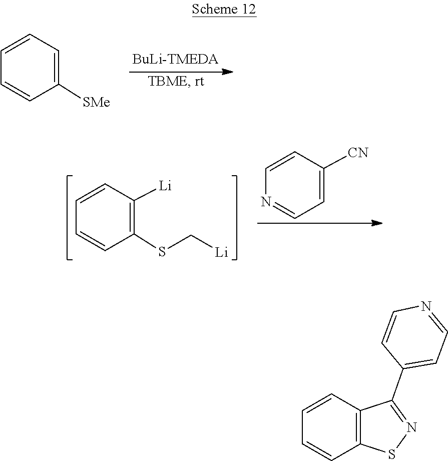

3-(Hetero)aryl-1,2-benzisoxazoles are readily available by an intramolecular cyclisation of oximes or imines derived from 2-halogeno- or 2-hydroxy-benzophenones (for a review see F. Gualtieri and M. Giannella in Isoxazoles eds. P. Grünanger and P. Vita Finzi, Chemistry of Heterocyclic Compounds, John Wiley & Sons Inc., 1999, vol. 48, part 2, p. 1). 3-(Pyridin-4-yl)-1,2-benzisoxazole was prepared via oxidative cyclisation of the imine derived from 4-(2-hydroxybenzoyl)pyridine with N-chlorosuccinimide (Scheme 13) according to the literature procedure (H. Hepburn and T. J. Donohoe, Chem. Eur. J., 2020, 26, 1963).

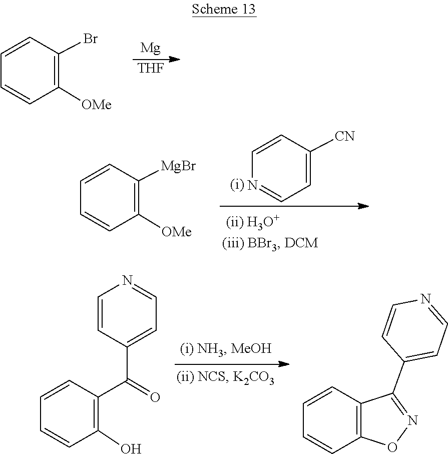

Electrochromic Composition

The present invention also relates to electrochromic compositions comprising at least one compound of formula (I) as defined above as an oxidizing electrochromic compound. One or more additional oxidizing electrochromic compounds can be added to the composition of the invention so as to adapt the colour or the intensity of the coloured state of the composition. Said additional compound can be another compound of formula (I) or a different compound such as compatible dyes or pigments. For example, the additional oxidizing electrochromic compound can be selected from alkylviologens, arylviologens, alkylarylviologens or anthraquinone and derivatives. Preferably, the additional compound has a redox potential close to the compound of formula (I).

The composition may also comprise at least one reducing compound. The reducing compound may also be an electrochromic compound. Example of reducing compounds include 5,10-dihydrophenazine, phenothiazine, phenoxazine, N,N,N′,N′-tetramethyl-p-phenylenediamine, thioanthrene, tetrathiafulvalene, ferrocene and their derivatives.

The composition of the invention may comprise a host medium that may be a fluid, a mesomorphous medium or a gel. The host medium is introduced in the composition of the invention to dissolve the electrochromic compounds. The host medium is preferably selected from the group consisting of organic solvents, liquid crystals, polymers, liquid crystal polymers and mixtures thereof.

Examples of suitable organic solvents that can be used as host medium are redox-compatible solvents which cannot react with the electrochromic compounds of the composition, such as ethylene carbonate, propylene carbonate, γ-butyrolactone, γ-valerolactone, acetronitrile, propionitrile, benzonitrile, glutaronitrile, methylglutaronitrile, dimethylformamide, N-methylpyrrolidone, sulfolane, 3-methyl sulfolane, benzene, toluene, methyl ethyl ketone, acetone, ethanol, tetrahydrofurfuryl alcohol, 2-methoxyethyl ether, xylene, cyclohexane, 3-methylcyclohexanone, ethyl acetate, ethyl phenylacetate, tetrahydrofuran, methanol, methyl propionate, ethylene glycol, ethylene carbonate, ionic liquids, and mixtures thereof. Preference is given to carbonates and particularly propylene carbonate.

Examples of suitable liquid crystals that can be used as host medium are nematic or chiral nematic media.

Examples of suitable polymers that can be used as host medium are polymers which are soluble with the solvent, in particular PMMA or other acrylate polymers, polyurethane, polyethylene oxide, polypropylene oxide, polyvinyl acetate, poly(N-vinyl pyrrolidone), and polyvinylidene fluoride.

Examples of suitable liquid crystal polymers that may be used as host medium are Merck RM257 (Merck), LC242 (BASF) or SLM 90519 (Wacker). These liquid crystal polymers are generally used in combination with an organic solvent, for example one of the organic solvents mentioned above.

### Electrochromic Device

The present invention also relates to an electrochromic device comprising a compound of formula (I) or a composition according to the invention. Said device may be selected from an optical article, preferably an optical lens, or an optical filter, a window, preferably an aircraft window, a visor, a mirror and a display, in particular a segmented or matrix display. Preferably, the device of the invention is an optical article, more preferably an optical lens, and even more preferably an ophthalmic lens.

Non-limiting examples of ophthalmic lens include corrective and non-corrective lenses, including single vision or multi-vision lenses, which may be either segmented or non-segmented, as well as other elements used to correct, protect, or enhance vision, including without limitation contact lenses, intra-ocular lenses, sunglasses, ski goggles, magnifying lenses, protective lenses and visors, for example motorcycle visors and helmets. Non-limiting examples of display elements and devices include screens and monitors. Non-limiting examples of windows include automotive, marine and aircraft windows, filters, shutters, and optical switches.

Preferably, the device of the invention comprises a mechanism for holding the compound or composition of the invention in a mechanically stable environment. More preferably, said device may comprise a pair of opposed substrates having a gap there between for receiving the mixture of the host medium and said compound or said composition of the present invention, and a frame for holding said pair of substrates adjacent one another.

The device of the present invention may thus comprise an optical component provided with at least one transparent cell arrangement juxtaposed in a parallel direction to the surface thereof, as disclosed in WO 2006/013250, each cell being tightly closed and containing at least one compound or composition of the present invention.

Other devices according to the invention can be devices as described in FR 2937154 or FR2950710 comprising at least one compound or composition of the invention.

### EXAMPLES

This invention will be further illustrated by the following non-limiting examples which are given for illustrative purposes only and should not restrict the scope of the appended claims.

### Synthesis of Compounds of the Invention

### 1. Iso)thiazole Benzologues

### Example 1

### 2-(Pyridin-4-yl)benzothiazole

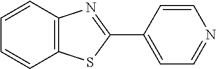 [SMILES: c1ccc2sc(-c3ccncc3)nc2c1]

A solution of 2-aminobenzenethiol (7.01 g, 56.1 mmol) and pyridine-4-carboxaldehyde (6.00 g, 56.1 mmol) in EtOH (28 mL) was stirred under air for 120 h. The resulting mixture was filtered, and the residue washed with cooled MeOH (20 mL) and air dried to give the title compound (7.63 g, 64%) as a cream powder. The liquors were reduced in volume and filtered to give a second crop (0.73 g, 6%), δ_{H} (CDCl₃, 400 MHz) 7.46 (1H, dt, J=1.2 and 8.2 Hz), 7.56 (1H, dt, J=1.2 and 8.2 Hz), 7.93-7.99 (3H, m), 8.14 (1H, ddd, J=8.2 Hz) and 8.78 (2H, dd, 1.6 and J=4.5 Hz); δ_{C} (CDCl₃, 100 MHz) 121.21, 121.89, 123.93, 126.22, 126.84, 135.21, 140.47, 150.79, 153.97 and 165.11.

### 4-(Benzothiazol-2-yl)-1-hexylpyridin-1-ium iodide

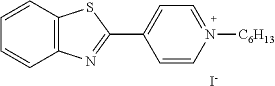

A solution of 2-(pyridin-4-yl)benzothiazole (0.54 g, 2.5 mmol) and 1-iodohexane (1.62 g, 7.6 mmol) in MeCN (40 mL) in the dark under N₂ was heated at reflux for 24 h, then cooled and diluted with Et₂O (50 mL). The precipitate was filtered off, washed with Et₂O (3×20 mL) and air dried to give the title compound (0.77 g, 71%) as a yellow powder, δ_{H} (DMSO-d₆, 400 MHz) 0.85 (3H, t, J=7.0 Hz), 1.21-1.37 (6H, m), 1.88-2.01 (2H, m), 4.65 (2H, t, J=7.3 Hz), 7.62-7.72 (2H, m), 8.25 (1H, dd, J=0.9 and 7.6 Hz), 8.35 (1H, dd, J=1.1 and 7.6 Hz), 8.76 (2H, d, 6.8 Hz) and 9.23 (2H, d, J=6.8 Hz); δ_{C} (DMSO-d₆, 100 MHz) 14.32, 22.34, 25.54, 31.06, 31.19, 61.11, 123.72, 124.82, 125.47, 128.23, 128.33, 136.64, 146.29, 146.73, 153.83 and 161.90.

### 4-(Benzothiazol-2-yl)-1-hexylpyridin-1-ium tetrafluoroborate

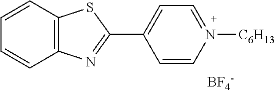

A solution of 4-(benzothiazol-2-yl)-1-hexylpyridin-1-ium iodide (0.77 g, 1.8 mol) in MeOH/H₂O (40 mL, 1:1) was added dropwise to a solution of NaBF₄ (3.96 g, 36 mmol) in water (80 mL) with stirring. Stirring was continued for 0.5 h and the resulting precipitate filtered, washed with water (2×5 mL) and air dried to give the title compound (0.69 g, 98%) as a pale yellow powder, δ_{H} (DMSO-d₆, 400 MHz) 0.88 (3H, t, J=6.9 Hz), 1.25-1.39 (6H, m), 1.89-2.02 (2H, m), 4.67 (2H, t, J=7.4 Hz), 7.64-7.75 (2H, m), 8.28 (1H, dd, J=1 and 8.3 Hz), 8.37 (1H, dd, J=1 and 7.6 Hz), 8.79 (2H, d, 6.8 Hz) and 9.23 (2H, d, J=6.8 Hz); δ_{C} (DMSO-d₆, 100 MHz) 13.75, 21.78, 24.98, 30.50, 30.63, 60.57, 123.14, 124.26, 124.90, 127.67, 127.77, 136.09, 145.72, 146.19, 153.27 and 161.33.

### Compound 1: 2-(1-Hexylpyridin-1-ium-4-yl)-3-methylbenzothiazol-3-ium bis(tetrafluoroborate)

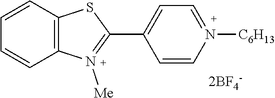

A mixture of 4-(benzothiazol-2-yl)-1-hexylpyridin-1-ium tetrafluoroborate (0.59 g, 1.5 mmol) in MeOTs (1.71 g) was heated at 180° C. for 2 h, cooled, triturated with Et₂O (3×40 mL) and dried under N₂ to give 0.81 g of a light tan powder. The solid was redissolved in MeOTs (1.71 g), heated at 180° C. for 1 h, cooled, triturated again with Et₂O (4×30 mL) and air dried. The gummy solid was dissolved in MeOH/water (25 mL, 1:4) and added dropwise to a solution of NaBF₄ (3.38 g, 30.7 mmol) in water (50 mL) with stirring. Stirring was continued for 0.5 h and the resulting precipitate filtered, washed with water (2×5 mL), air dried and triturated with hot MeOH (8 mL). The residue was cooled, filtered and washed with cold MeOH (2 mL) to give the title compound (0.38 g, 51%) as a cream powder, δ_{H} [(CD₃)₂CO, 400 MHz] 0.91 (3H, t, J=7.0 Hz), 1.29-1.47 (4H, m), 1.49-1.61 (2H, m), 2.20-2.32 (2H, m), 4.58 (3H, s), 5.04 (2H, t, J=7.7 Hz), 8.07 (1H, t, J=7.9 Hz), 8.17 (1H, t, J=7.8 Hz), 8.58 (1H, d, J=8.4 Hz), 8.68 (1H, d, J=8.4 Hz), 8.89 (2H, bd, J=5.7 Hz) and 9.59 (2H, d, J=6.5 Hz); δ_{F} [(CD₃)₂CO, 376 MHz]-151.40, −151.35; δ_{C} [(CD₃)₂CO, 100 MHz] 13.30, 22.14, 25.54, 30.98, 31.37, 38.42, 62.91, 117.97, 124.70, 129.92, 130.08, 131.05, 131.42, 141.05, 142.81, 146.47 and 168.29.

### Example 2

### 4-(Benzothiazol-2-yl)-1-phenylpyridin-1-ium triflate

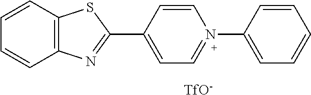

A mixture of 2-(pyridin-4-yl)benzothiazole (1.34 g, 6.3 mmol), diphenyliodonium triflate (3.47 g, 8.1 mmol), Cu(OAc)₂·H₂O (0.11 g, 0.55 mmol, 10 mol %) in dry DMF (25 mL) was heated at 100° C. for 16 h, cooled and the solvent removed under reduced pressure. The residue was triturated with Et₂O (3×40 mL) and air dried to give the title compound (2.66 g, 96%) as a yellow powder, δ_{H} [(CD₃)₂CO, 400 MHz] 7.67-7.86 (5H, m), 8.01-8.09 (2H, m), 8.27-8.37 (2H, m), 9.02 (2H, d, 6.8 Hz) and 9.56 (2H, d, J=6.8 Hz); δ_{F}[(CD₃)₂CO, 376 MHz]−77.76; δ_{C} (DMSO-d₆, 100 MHz) 122.93, 124.60, 124.85, 125.25, 128.01, 128.18, 130.60, 131.78, 136.92, 142.95, 142.95, 145.92, 148.33, 154.18 and 160.76.

### Compound 2: 3-Methyl-2-(1-phenylpyridin-1-ium-4-yl)benzothiazol-3-ium bis(tetrafluoroborate)

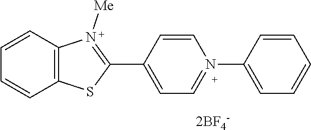

A mixture of 4-(benzothiazol-2-yl)-1-phenylpyridin-1-ium triflate (1.07 g, 2.4 mmol) MeOTs (3.62 g, 19 mmol) was heated at 180° C. for 3 h, cooled and triturated with Et₂O (3×40 mL) and the residue dried under vacuum. The residue was dissolved in MeOH (5 mL) and added dropwise to a solution of NaBF₄ (5.37 g, 48.8 mmol) in water (40 mL) with stirring. Stirring was continued for 0.5 h and the resulting precipitate was filtered, washed with water (2×5 mL) and air dried. The residue was triturated with hot MeOH (2×20 mL), filtered and air dried to give the title compound (0.72 g, 62%) as a grey powder, δ_{H} (DMSO-d₆, 400 MHz) 4.32 (3H, s), 7.80-7.90 (3H, m), 7.92-7.99 (2H, m), 8.04 (1H, t, J=7.8 Hz), 8.14 (1H, t, J=7.5 Hz), 8.55 (1H, d, J=8.6 Hz), 8.69 (1H, d, J=8.2), 8.79 (2H, d, J=6.7 Hz) and 9.77 (2H, d, J=6.7 Hz); δ_{F} (DMSO-d₆, 376 MHz) −148.23, −148.18; δ_{C} (DMSO-d₆, 100 MHz) 118.42, 125.21, 125.57, 129.92, 130.10, 130.99, 131.21, 131.61, 132.40, 141.77, 142.47, 142.92, 146.55 and 168.49.

### Example 3

### (E)-3,4-Dihydronaphthalen-1(2H)-one O-acetyl oxime

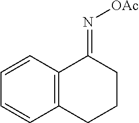 [SMILES: CC(=O)O/N=C1\CCCc2ccccc21]

Solid hydroxylamine hydrochloride (7.14 g, 103 mmol) was added in one portion to a solution of 3,4-dihydronaphthalen-1(2H)-one (10.00 g, 68.5 mmol) in EtOH (30 mL). The resulting solution was heated at 60° C. for 1 h, poured into HCl (200 mL, 2 M) and extracted with EtOAc (3×50 mL). The combined organic extracts were washed with brine (50 mL), then water (50 mL) and dried (Na₂SO₄) and the solvent removed under reduced pressure. The residue was dissolved in pyridine (32 mL) to which Ac₂O (13.97 g, 137 mmol) and DMAP (16 mg) were then added. The resulting solution was stirred at rt for 1 h, poured into HCl (300 mL, 2 M) and extracted with EtOAc (3×200 mL). After drying (Na₂SO₄) the solvent was removed under reduced pressure. The residue was twice crystallized from EtOAc/hexanes to give the title compound (10.51 g, 76%) as colourless needles, δ_{H} (CDCl₃, 400 MHz) 1.87-1.93 (2H, m), 2.27 (3H, s), 2.77-2.82 (2H, br. t, J=6.1 Hz), 2.86-2.90 (2H, m), 7.16-7.20 (1H, m), 7.22-7.26 (1H, m), 7.34 (1H, dt, J=1.4 and 7.4 Hz) and 8.13-8.16 (1H, m); δ_{C} (CDCl₃, 100 MHz) 19.92, 21.33, 25.60, 29.55, 125.61, 126.59, 128.72, 128.95, 130.74, 140.93, 161.31 and 169.22.

### 2-(Pyridin-4-yl)naphtho[1,2-d]thiazole

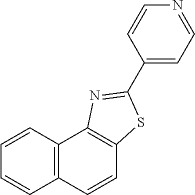 [SMILES: c1ccc2c(c1)ccc1sc(-c3ccncc3)nc12]

A mixture of (E)-3,4-dihydronaphdialen-1(2H-one O-acetyl oxime (4.00 g, 19.7 mmol), pyridine-4-carboxaldehyde (1.40 g, 13.1 mmol) and sulfur (10.09 g, 39.4 mmol) in DMSO (60 mL) under N₂ was heated at 120° C. for 4 h, poured into water (200 mL) and extracted with EtOAc (3×100 mL). The combined extracts were washed with water (100 mL), dried (Na₂SO₄) and the solvent removed under reduced pressure. The residue was chromatographed on silica using EtOAc (25-30% in hexanes gradient) as eluent. The fluorescent band was collected and the solvent removed under reduced pressure. The residue was crystallised from hot EtOAc/hexanes at 0° C. to give the title compound (1.14 g, 33%) as brown needles, δ_{H} (CDCl₃, 600 MHz) 7.64 (1H, ddd, J=1.3, 6.9 and 8.1 Hz), 7.74 (1H, ddd, J=1.2, 6.9 and 8.2 Hz), 7.87-7.90 (1H, br. dm, J=8.7 Hz), 7.95 (1H, d, J=8.7 Hz), 7.98-8.01 (1H, br. m, J=8.1 Hz), 8.06 (2H, d, J=6.0 Hz), 7.80 (2H, d, J=6.0 Hz) and 8.93 (1H, br. d, J=8.2 Hz); δ_{C} (CDCl₃, 125 MHz) 118.90, 120.98, 123.96, 126.61, 127.17, 127.40, 128.22, 128.92, 132.16, 132.33, 140.77, 150.56, 150.78 and 163.88.

### 4-(Naphtho[1,2-d]thiazol-2-yl)-1-phenylpyridin-1-ium trifluoromethanesulfonate

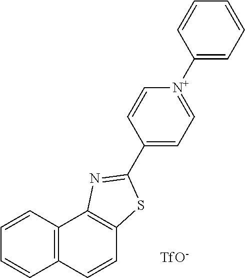

A mixture of 2-(pyridin-4-yl)naphtho[1,2-d]thiazole (1.12 g, 4.3 mmol), diphenyliodonium trifluoromethanesulfonate (2.20 g, 5.1 mmol) and Cu(OAc)₂·H₂O (86 mg, 10 mol %) in DMF (30 mL) under N₂ was heated at 100° C. for 16 h. The resulting solution was cooled and the solvent removed under reduced pressure. The residue was triturated with Et₂O (3×30 mL) and air dried to give the title compound (2.09 g, 100%) as a brown powder which was used without further purification in the next step, δ_{H} (DMSO-d₆, 400 MHz) 7.71-7.91 (5H, m), 7.93-8.01 (2H, m), 8.16-8.23 (2H, m), 8.40 (1H, d, J=8.9 Hz), 8.92 (1H, d, J=8.1 Hz), 8.96 (2H, d, J=7.0 Hz) and 9.49 (2H, d, J=7.0 Hz); δ_{F} (DMSO-d₆, 376 MHz) −77.75.

### Compound 3: 1-Methyl-2-(1-phenylpyridin-1-ium-4-yl)naphtho[1,2-d]thiazol-1-ium bis(tetrafluoroborate)

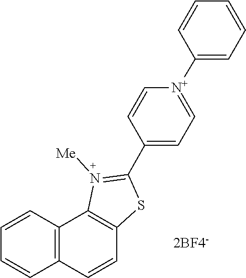

A solution of 4-(naphtho[1,2-d]thiazol-2-yl)-1-phenylpyridin-1-ium trifluoromethanesulfonate (2.09 g, 4.3 mmol) in methyl trifluoromethanesulfonate (9.03 g, 55.1 mmol) was heated at reflux. After 2 days the solution was diluted with Et₂O (40 mL) and the residue collected by filtration. The filtrand was washed with Et₂O (2×10 mL) and air dried. The solid was extracted with water (2×100 mL) and the solvent removed under reduced pressure. The residue was then dissolved in MeOH (5 mL) and added dropwise to a solution of NaBF₄ (5.05 g, 45.9 mmol) in water (40 mL) with stirring. Stirring was continued for 0.5 h and the resulting precipitate filtered, washed with water (3×5 mL) and triturated with hot MeOH (20 mL). After cooling, the residue was filtered off and air dried to give the title compound (0.23 g, 8%) as a pale yellow solid. δ_{H} (DMSO-d₆, 400 MHz) 4.74 (3H, s), 7.76-8.12 (7H, m), 8.40-8.70 (3H, br. m), 8.82 (2H, br. d, J=3.3 Hz), 9.11 (1H, d. dm, J=9.0 Hz) and 9.79 (2H, br. d, J=3.3 Hz); δ_{F} (DMSO-d₆, 376 MHz) −148.25 and −148.20.

### Example 4

### Quinoline-4-carbaldehyde

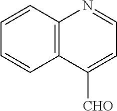 [SMILES: O=Cc1ccnc2ccccc12]

A mixture of 4-methylquinoline (6.00 g, 41.9 mmol), p-toluenesulfonic acid (7.22 g, 42 mmol) and iodine (4.26 g, 16.8 mmol, 40 mol %) in DMSO (300 mL) was heated at 130° C. for 16 h, cooled, poured into water (400 mL) and basified (K₂CO₃). The mixture was extracted with EtOAc (5×100 mL). The combined organic extracts were washed with aq. Na₂S₂O₃ (300 mL), then brine (200 mL) and dried (Na₂SO₄). The solvent was removed under reduced pressure to give the title compound (6.11 g, 93%) as a beige solid, δ_{H} (CDCl₃, 400 MHz) 7.75 (1H, ddd, J=1.4, 6.9 and 8.4 Hz), 7.80 (1H, d, J=4.3 Hz), 7.83 (1H, d, J=1.4, 6.9 and 8.4), 8.23 (1H, ddd, J=0.7, 1.4 and 8.4 Hz), 9.11 (1H, ddd, J=0.7, 1.4 and 8.4 Hz), 9.21 (1H, d, J=4.3 Hz) and 10.53 (1H, s); δ_{C} (CDCl₃, 100 MHz) 123.89, 124.44, 125.87, 129.42, 130.06, 130.22, 136.76, 149.28, 150.48 and 192.92.

### 2-(Quinolin-4-yl)-2,3-dihydrobenzothiazole

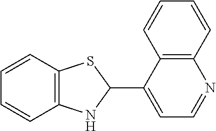 [SMILES: c1ccc2c(c1)NC(c1ccnc3ccccc13)S2]

A solution of quinoline-4-carbaldehyde (3.00 g, 19.1 mmol) and 2-aminobenzenethiol (2.39 g, 19.1 mmol) in EtOH (40 mL) was stirred under air for 2 days. The solvent was decanted and the residue crystallised from hot EtOH (40 mL) and cooled to 0° C., The product was collected by filtration, washed with EtOH (10 mL) and air dried to give the title compound (2.38 g, 47%) as orange prisms. The filtrate liquors were reduced to give a second crop (1.19 g, 23%) as a yellow powder, δ_{H} (DMSO-d₆, 400 MHz) 6.62 (1H, dt, J=1.2 and 7.5 Hz), 6.78 (1H, br. dd, J=1.1 and 7.8 Hz), 6.94 (1H, dt, J=1.3 and 7.7 Hz), 6.99 (1H, dd, J=1.2 and 7.4 Hz), 7.15 (2H, s), 7.62 (1H, d, J=4.5 Hz), 7.67 (1H, ddd, J=1.4, 6.9 and 8.4 Hz) 7.79 (1H, ddd, J=1.4, 6.9, 8.4 Hz) 8.07 (2H, br. t, J=8.8 Hz) and 8.88 (1H, d, J=4.4 Hz); δ_{C} (DMSO-d₆, 100 MHz) δ4.46, 109.88, 117.60, 119.59, 121.94, 123.84, 124.74, 125.14, 126.21, 127.39, 129.94, 130.26, 148.02, 148.16, 148.40 and 151.08.

### 2-(Quinolin-4-yl)benzothiazole

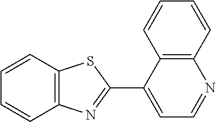 [SMILES: c1ccc2sc(-c3ccnc4ccccc34)nc2c1]

Solid DDQ (1.29 g, 5.7 mmol) was added to a solution of 2-(quinolin-4-yl)-2,3-dihydrobenzothiazole (1.50 g, 5.7 mmol) in DCM (1.2 L). The mixture was stirred at rt for 1 h, then filtered through silica using EtOAc (60% in hexanes) as eluent. The solvent was removed under reduced pressure and the residue dissolved in EtOAc (200 mL) and washed sequentially with KOH (200 mL, 2 M), water (2×100 mL) and brine (100 mL). The extracts were dried (Na₂SO₄) and the solvent removed under reduced pressure. The residue was crystallised from hot EtOAc/hexanes. After cooling to −4° C., the product was collected by vacuum filtration and washed with cold hexanes to give the title compound (1.19 g, 80%) as pale lime needles, δ_{H} (CDCl₃, 400 MHz) 7.52 (1H, ddd, J=1.3, 7.3 and 7.9 Hz), 7.60 (1H, ddd, J=1.3, 7.2 and 8.2 Hz), 7.70 (1H, ddd, J=1.3, 6.8 and 8.7 Hz), 7.80 (1H, d, J=4.4 Hz), 7.82 (1H, ddd, J=1.4, 6.9 and 8.4 Hz), 8.02 (1H, ddd, J=0.7, 1.3 and 8.0 Hz), 8.22 (1H, ddd, J=0.7, 1.4 and 8.4 Hz) 8.24 (1H, ddd, J=0.6, 1.2 and 8.2 Hz), 9.00 (1H, ddd, J=0.7, 1.4 and 8.5 Hz) and 9.05 (1H, d, J=4.4 Hz); δ_{C} (CDCl₃, 100 MHz) 121.63, 122.19, 124.12, 124.98, 126.04, 126.17, 126.76, 128.19, 130.00 (2×C), 135.34, 138.32, 149.20, 149.80, 154.18, 164.80.

### 4-(Benzothiazol-2-yl)-1-phenylquinolin-1-ium tetrafluoroborate

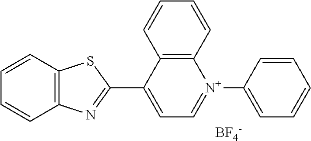

A mixture of 2-(quinolin-4-yl)benzothiazole (1.00 g, 3.8 mmol), diphenyliodonium trifluoromethanesulfonate (2.46 g, 5.7 mmol) and Cu(OAc)₂·H₂O (76 mg, 10 mol %) in DMF (30 mL) under N₂ was heated at 100° C. for 16 h. The resulting solution was cooled and the solvent removed under reduced pressure, the residue was triturated with Et₂O (3×40 mL) to give a mixture (62:38) of product and starting material. The residue and diphenyliodonium trifluoromethanesulfonate (1.64 g, 3.8 mmol) and Cu(OAc)₂·H₂O (76 mg, 10 mol %) in DMF (30 ml) under N₂ was heated at 100° C. for 16 h. The resulting solution was cooled and the solvent removed under reduced pressure. The residue was triturated with Et₂O (3×40 mL) to give a mixture of product and reactant (70:30). This solid was dissolved in warm MeOH (20 mL) and added dropwise to a solution of NaBF₄ (8.40 g, 76 mmol) in water (50 mL) with stirring. Stirring was continued for 0.5 h and the resulting precipitate collected by filtration and washed with water (5 mL). The solid was dissolved in hot MeOH (20 mL) and added dropwise to NaBF₄ (8.40 g, 76 mmol) in water (50 mL) with stirring. Stirring was continued for 0.5 h and the resulting precipitate filtered off, washed with water (5 mL) and air dried to give a yellow powder. The powder was triturated with hot EtOH (2×5 mL) to give the title compound (1.01 g, 54%) as a yellow powder, δ_{H} (CDCl₃, 400 MHz) 7.67-7.79 (2H, m), 7.79-8.92 (6H, m), 8.12-8.24 (2H, m), 8.27 (1H, br. d, J=7.6 Hz), 8.38 (1H, br. d, J=7.9 Hz), 8.68 (1H, d, J=6.2 Hz), 9.52 (1H, d, J=6.2 Hz) and 9.69 (1H, br. dd, J=1.5 and 8.3 Hz); δ_{F} (CDCl₃, 376 MHz) −154.67 and −154.62; Sc (CDCl₃, 100 MHz) 120.31, 122.03, 122.56, 124.59, 126.34, 126.89, 127.61, 127.76, 128.91, 130.47, 131.13, 131.79, 135.76, 136.23, 140.35, 140.92, 148.20, 149.02, 154.37 and 161.44.

### Compound 4: 3-Methyl-2-(1-phenylquinolin-1-ium-4-yl)benzothiazol-3-ium tetrafluoroborate

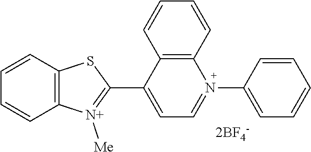

A mixture of 4-(benzothiazol-2-yl)-1-phenylquinolin-1-ium tetrafluoroborate (1.01 g, 2.4 mmol) in MeOTs (4.61 g, 24.8 mmol) was heated at 180° C. for 3 h, then cooled and triturated with Et₂O (3×30 mL). The washings were discarded and the residue then dried under N₂. The residue was dissolved in hot MeOH (40 mL) and added dropwise to a solution of NaBF₄ (4.55 g, 41.4 mmol) in water (40 mL) with stirring. Stirring was continued for 0.5 h and the precipitate collected by filtration and washed with water (2×5 mL), and then with hot MeOH (5 mL) and air dried. The residue was triturated with AcMe (3 mL) to give the title compound (0.70 g, 56%) as an off white powder, δ_{H} [(CD₃)₂CO, 600 MHz] 4.52 (3H, s), 7.88-8.02 (5H, m), 8.07 (1H, app. br. d, J=9.0 Hz), 8.12 (1H, ddd, J=0.7, 7.3 and 8.7 Hz), 8.19-8.24 (2H, m), 8.39 (1H, ddd, J=1.3, 7.0 and 9.0 Hz), 8.57 (1H, ddd, J=0.6, 1.3 and 8.5 Hz), 8.64 (1H, ddd, J=0.7, 1.6, 7.9 Hz), 8.75 (1H, ddd, J=0.6, 1.2 and 7.3 Hz), 9.00 (1H, d, J=5.9 Hz) and 9.97 (1H, d, J=5.9 Hz); δ_{F} [(CD₃)₂CO, 470 MHz] −151.44 and −151.39; δ_{C} [(CD₃)₂CO, 125 MHz] 38.76, 118.09, 121.40, 124.76, 125.84, 126.59, 127.07, 128.87, 130.00, 130.73, 131.06, 132.25, 132.32, 132.52, 137.16, 140.39, 140.69, 141.56, 142.98, 150.58 and 166.95.

### Example 5

### 3-(Pyridin-4-yl)-1,2-benzisothiazole

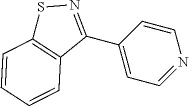 [SMILES: c1ccc2c(-c3ccncc3)nsc2c1]

“BuLi (2.5 M in hexane) (32.2. mL, 80.5 mmol) was added dropwise to a stirred solution of thioanisole (2.00 g, 16.1 mmol) and N,N,N′,N′-tetramethylethylenediamine (5.60 g, 7.24 mL, 48.3 mmol) in tert-butyl methyl ether (80 mL) under N₂. The reaction mixture was then stirred under N₂ at room temperature for 3 h. 4-Pyridinecarbonitrile (16.76 g, 161 mmol) was then added portion wise and the reaction mixture was then stirred under N₂ at room temperature for 24 h. The reaction mixture was then carefully quenched with sat. NH₄Cl (100 mL) and extracted with DCM (3×200 mL). The organic layers were combined, dried (Na₂SO₄), filtered and the solvent was removed under reduced pressure. The resulting residue was chromatographed on silica gel [eluent=1:9 graduated to 4:6 EtOAc:DCM]. The solvent of the resulting column fractions was removed under reduced pressure and the resulting residue was again chromatographed on silica gel [eluent=1:9 graduated to 4:6 EtOAc:DCM]. The solvent of the resulting column fractions was removed under reduced pressure and the resulting residue was recrystallised from hot petroleum ether and filtered to give the title compound (0.33 g, 15%) as colourless crystals. The filtrate from the recrystallisation was left to stand at −20° C. for 16 h whereupon a second crop was obtained (0.15 g, 7%) as colourless crystals. Yield 0.48 g, 22%. δ_{H} (CDCl₃, 400 MHz) 8.81 (2H, d, J=5.3 Hz), 8.18 (1H, d, J=8.2 Hz), 8.02 (1H, d, J=8.2 Hz), 7.79 (2H, d, J=5.9 Hz), 7.59 (1H, t, J=7.3 Hz) and 7.50 (1H, t, J=7.3 Hz); δ_{C} (CDCl₃, 100 MHz) 161.4, 153.9, 150.5, 142.2, 133.36, 127.9, 125.6, 124.1, 123.0 and 120.2.

### Compound 5: 4-(1,2-Benzisothiazol-3-yl)-1-phenylpyridin-1-ium tetrafluoroborate

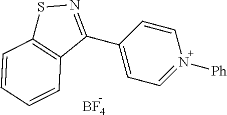

3-(Pyridin-4-yl)-1,2-benzisothiazole (0.36 g, 1.7 mmol), diphenyliodonium trifluoromethanesulfonate (1.12 g, 2.6 mmol) and Cu(OAc)₂. H₂O (34 mg, 0.17 mmol) were dissolved in DMF (20 mL) under N₂ and the reaction mixture was stirred at 100° C. for 16 h. The reaction mixture was cooled to room temperature and the solvent was removed under reduced pressure. The resulting pale yellow solid was triturated with diethyl ether (3×50 mL), filtered and dried under reduced pressure. The resulting pale yellow solid was dissolved in hot MeOH (150 mL) and added dropwise through a cotton wool plug to a stirred solution of NaBF₄ (15.0 g) in H₂O (300 mL) whereupon a pale yellow precipitate formed. The suspension was stirred for 30 minutes and then filtered under reduced pressure. The resulting solid was washed with water (30 mL) and then dried under reduced pressure to give a pale yellow powder. The solid was then dissolved in hot acetone and added dropwise through a cotton wool plug back into the filtrate (which had been reduced by ca. 30% under vacuum) to give a pale yellow precipitate. The resulting suspension was filtered under reduced pressure and the solid so obtained, washed with water (30 mL) and then dried under reduced pressure to give the title compound as a pale yellow powder. Yield 0.38 g, 59%. δ_{H} (DMSO-d₆, 400 MHz) 9.55 (2H, d, J=6.9 Hz), 8.84 (2H, d, J=6.9 Hz), 8.52 (2H, d, J=8.9 Hz), 8.07-7.95 (2H, m) and 7.92-7.71 (5H, m); δ_{C} (DMSO-d₆, 100 MHz) 157.4, 154.0, 148.8, 145.6, 142.5, 132.8, 131.4, 130.3, 128.6, 126.8, 126.6, 124.8, 123.9 and 121.5; δ_{F} (DMSO-d₆, 376 MHz) −148.22 (4F, br. m).

### Example 6

### Compound 6: 2-Methyl-3-(1-phenylpyridin-1-ium-4-yl)-1,2-benzisothiazol-2-ium bis(hexafluorophosphate)

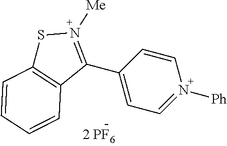

4-(1,2-Benzisothiazol-3-yl)-1-phenylpyridin-1-ium tetrafluoroborate (0.30 g, 0.8 mmol) was suspended in methyl trifluoromethanesulfonate (0.8 g, 5.4 mmol) and the reaction mixture was stirred under N₂ at 100° C. for 80 minutes. The reaction mixture was then stirred under N₂ at 40° C. for 4 h. After cooling to room temperature, diethyl ether (50 mL) was added and the resulting suspension was filtered to give a tan powder that was then triturated with ether (3×50 mL) and dried under reduced pressure. This tan powder was then dissolved in hot MeOH (50 mL) and added dropwise through a cotton wool plug to a stirred solution of NH₄PF₆ (10.0 g) in H₂O (200 mL) whereupon a tan coloured precipitate formed. The precipitate was then washed with water (30 mL) and then dried under reduced pressure to give a pale tan powder. The pale tan powder was then recrystallised from hot MeOH to give the title compound as a pale tan powder Yield 0.18 g, 38%. δ_{H} (DMSO-d₆, 400 MHz) 9.81 (2H, d, J=6.9 Hz), 8.75 (2H, d, J=6.9 Hz), 8.71 (1H, d, J=6.6 Hz), 8.18 (1H, ddd, J=8.5, 7.3, 0.9 Hz), 8.10 (1H, d, J=6.6 Hz), 8.05-7.82 (6H, m) and 4.33 (3H, s); δ_{C} (DMSO-d₆, 100 MHz) 159.3, 147.8, 146.2, 142.5, 142.4, 134.5, 131.9, 130.9, 130.5, 129.3, 128.8, 127.0, 124.7, 122.9 and 39.5; δ_{F} (DMSO-d₆, 376 MHz) −70.14 (12F, d, J=711.5 Hz).

### 2. (Benz)Imidazoles and Fused-Ring Derivatives

### Example 7

### 2-(Pyridin-4-yl)-1H-benzimidazole

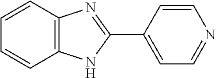 [SMILES: c1ccc2[nH]c(-c3ccncc3)nc2c1]

A solution of o-phenylenediamine (10.10 g, 93.5 mmol) and pyridine-4-carboxaldehyde (10.00 g, 93.5 mmol) in EtOH (500 mL) was stirred under air for 72 h and the solvent removed under reduced pressure. The residue was crystallised from EtOAc/hexanes then triturated with hot EtOAc three times to give the title compound (11.44 g, 63%) as a tan powder, δ_{H} (DMSO-d₆, 400 MHz) 7.26 (2H, bs), 7.59 (1H, vbs), 7.71 (1H, vbs), 8.80 (2H, d, J=6.1 Hz), 8.75 (2H, d, J=6.1 Hz) and 13.26 (1H, bs).

### 1-Methyl-2-(pyridin-4-yl)-1H-benzimidazole

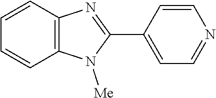 [SMILES: Cn1c(-c2ccncc2)nc2ccccc21]

A mixture of 2-(pyridin-4-yl)-1H-benzimidazole (5.33 g, 27.3 mmol), Mel (5.33 mL, 12.15 g, 85.6 mmol) and KOH (7.83 g, 140 mmol) in acetone (660 mL) was stirred at rt for 2 h and poured into PhMe (700 mL). The resulting solution was washed with water (1 L), brine (100 mL), dried (Na₂SO₄) and the solvent removed under reduced pressure. The residue was filtered through neutral alumina using EtOAc as eluent. The solvent was removed under reduced pressure and the residue crystallised from EtOAc/hexanes to give the title compound (2.80 g, 49%) as yellow plates, δ_{H} (CDCl₃, 400 MHz) 3.83 (3H, s), 7.72-7.46 (3H, m), 7.72 (2H, d, J=6.0 Hz), 7.83-7.87 (1H, m) and 8.06 (2H, d, J=6.0 Hz); δ_{C} (CDCl₃, 100 MHz) 31.84, 109.88, 120.37, 123.03, 123.45, 123.74, 136.74, 137.86, 142.91, 150.38 and 150.76.

### 1-Hexyl-4-(1-methyl-1H-benzimidazol-2-yl)pyridin-1-ium tetrafluoroborate

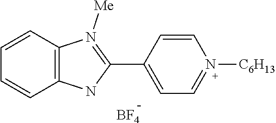

A solution of 1-methyl-2-(pyridin-4-yl)-1H-benzimidazole (1.47 g, 7 mmol) and 1-iodohexane (4.47 g, 21.1 mmol) in MeCN (40 mL) was heated at reflux in the dark under N₂ with stirring. After 24 h, the resulting mixture was cooled, diluted with Et₂O (60 mL), filtered, washed with Et₂O (30 mL) and dried under vacuum. The hygroscopic orange powder was dissolved in MeOH (30 mL) and added dropwise to a solution of NaBF₄ (4.55 g, 41.4 mmol) in water (200 mL) with stirring. Stirring was continued for 0.5 h and the resulting precipitate was filtered, washed with water (3×10 mL) and air dried to give the title compound (1.91 g, 73%) as a pale yellow powder, δ_{H} [(CD₃)₂CO, 400 MHz] 0.88 (3H, t, J=7.3 Hz), 1.27-1.56 (6H, m), 2.15-2.27 (2H, m), 4.23 (3H, s), 4.93 (2H, t, J=7.7 Hz), 7.39 (1H, ddd, J=1.1, 7.1 and 8.2 Hz), 7.48 (1H, ddd, J=1.2, 7.1 and 8.2 Hz), 7.70-7.74 (1H, m), 7.80-7.84 (1H, m), 8.80 (2H, d, J=6.9 Hz) and 9.34 (2H, d, J=6.9 Hz Hz); δ_{F} [(CD₃)₂CO, 376 MHz]-151.70 and −151.54; δ_{C} [(CD₃)₂CO, 100 MHz] 13.31, 22.17, 25.54, 31.00, 31.24, 32.08, 61.60, 111.15, 120.61, 123.44, 125.00, 127.04, 137.95, 143.21, 145.02, 145.87 and 147.05.

### Compound 7: 2-(1-Hexylpyridin-1-ium-4-yl)-1,3-dimethyl-1H-benzimidazol-3-ium bis(tetrafluoroborate)

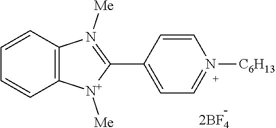

A mixture of 1-hexyl-4-(1-methyl-1H-benzimidazol-2-yl)pyridin-1-ium tetrafluoroborate (0.50 g, 1.3 mmol) in MeOTs (4.00 g, 21.5 mmol) was heated at 180° C. for 90 min, cooled and then triturated with Et₂O (3×30 mL). The residue was dissolved in MeOH (20 mL) and added drop wise to a solution of NaBF₄ (20.97 g, 190 mmol) in water (100 mL) with stirring. Stirring was continued for 0.5 h and the resulting precipitate filtered, washed with water (2×3 mL) and air dried to give 2-(1-hexylpyridin-1-ium-4-yl)-1,3-dimethyl-1H-benzimidazol-3-ium bis(tetrafluoroborate) (0.45 g, 71%) as a grey powder, δ_{H} (CD₃OD, 400 MHz) 0.91-0.99 (3H, m), 1.35-1.59 (6H, m), 2.10-2.22 (2H, m), 4.07 (6H, s), 4.81 (2H, t, J=7.6 Hz), 7.81-7.88 (2H, m), 8.05-8.12 (2H, m), 8.61 (2H, d, J=5.4 Hz) and 9.43 (2H, d, J=5.4 Hz); δ_{F} (DMSO-d₆, 376 MHz) −148.25; Sc (DMSO-d₆, 100 MHz) 14.34, 22.34, 25.60, 31.14, 31.37, 33.44, 62.15, 114.19, 128.06, 130.84, 132.49, 136.91, 145.57 and 146.60.

### Example 8

### Compound 8: 2-(1-hexylpyridin-1-ium-4-yl)-1,3-dimethyl-1H-benzimidazol-3-ium bis(hexafluorophosphate)

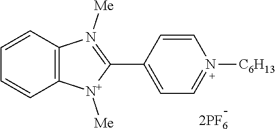

Solid ammonium hexafluorophosphate (1.16 g, 7.1 mmol) was added to the aqueous filtrate and washings obtained during the preparation of compound 3 above and the resulting precipitate filtered, washed with water (2×2 mL), triturated with hot MeOH (2 mL) and air dried to give 2-(1-hexylpyridin-1-ium-4-yl)-1,3-dimethyl-1H-benzimidazol-3-ium bis(hexafluorophosphate) (0.21 g, 27%) as a cream powder; δ_{H} (DMSO-d₆, 400 MHz) 0.89 (3H, t, J=6.8 Hz), 1.25-1.47 (6H, m), 1.94-2.08 (2H, m), 3.97 (6H, s), 4.74 (2H, m), 7.81-7.88 (2H, m), 8.15-8.23 (2H, m), 8.63 (2H, d, J=6.4 Hz) and 9.48 (2H, d, J=6.4 Hz); δ_{F} (DMSO-d₆, 376 MHz) −70.13 (d, J=710 Hz); δ_{C} (DMSO-d₆, 100 MHz) 14.34, 22.35, 25.60, 31.14, 31.40, 33.47, 62.14, 114.20, 128.09, 130.87, 132.48, 136.90, 145.57 and 146.61.

### Example 9

### 1-Hexyl-2-(pyridin-4-yl)-1H-benzimidazole

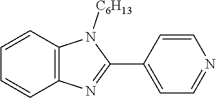

A mixture of 2-(pyridin-4-yl)-1H-benzimidazole (2.00 g, 10.3 mmol), powdered KOH (0.92 g, 16.4 mmol) and 1-iodohexane (2.39 g, 11.3 mmol) in anhydrous DMSO (30 mL) under N₂ was stirred at rt for 24 h then poured into water (100 ml) and extracted with DCM (3×100 mL). The combined extracts were washed with water (100 mL), dried (Na₂SO₄) and the solvent removed under reduced pressure. The residue was filtered through silica using EtOAc as eluent. The solvent was removed under reduced pressure and the residue triturated with hexanes (3×50 mL), filtered, washed with hexanes and air dried to give the title compound (2.53 g, 91%) as a tan powder, δ_{H} (CDCl₃, 400 MHz) 0.77-0.87 (3H, m), 1.17-1.35 (6H, m), 1.83-1.96 (2H, m), 4.450 (2H, t, J=7.6), 7.47-7.66 (3H, m), 7.90 (2H, d, J=6 Hz), 7.94 (1H, d, J=8 Hz) and 8.93 (2H, d, J=6 Hz); δ_{C} (CDCl₃, 100 MHz) 13.85, 22.36, 26.26, 29.70, 30.99, 46.00, 111.72, 118.10, 124.02, 126.43, 126.58, 133.56, 134.98, 135.17, 147.07 and 149.00.

### Compound 9: 3-Hexyl-1-methyl-2-(1-methylpyridin-1-ium-4-yl)-1H-benzimidazol-3-ium bis(hexafluorophosphate)

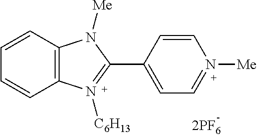

A mixture of 1-hexyl-2-(pyridin-4-yl)-1H-benzimidazole (0.51 g, 1.84 mmol) in MeOTs (1.71 g, 9.2 mmol) was heated at 180° C. for 3 h, cooled, triturated with Et₂O (3×40 mL) and the residue air dried. The residue was dissolved in MeOH (8 mL) and added dropwise to a solution of NaBF₄ (8.10 g, 73.6 mmol) in water (100 mL) with stirring. Stirring was continued for 0.5 h. Ammonium hexafluorophosphate (10 g, 61 mmol) was added and stirring continued for 0.5 h. The resulting precipitate was filtered, washed with water (2×10 mL), triturated with hot MeOH (40 mL), cooled, filtered, washed with MeOH (5 mL) and air dried to give the title compound (0.96 g, 87%) as a colourless powder, δ_{H} [(CD₃)₂CO, 400 MHz] 0.83 (3H, t, J=6.7), 1.17-1.45 (6H, m), 1.93-2.02 (2H, m), 4.17 (3H, s), 4.63 (2H, t, J=7.7 Hz), 4.84 (3H, s), 7.87-7.95 (2H, m), 8.17-8.33 (2H, m), 8.98 (2H, d, J=6 Hz) and 9.61 (2H, d, J=6 Hz); δ_{F} [(CD₃)₂CO, 376 MHz]-72.687 (d, J=710 Hz); δ_{C} [(CD₃)₂CO, 100 MHz] 13.20, 22.19, 25.80, 29.44, 31.07, 32.82, 47.06, 49.33, 99.81, 113.72, 113.86, 128.01, 130.43, 131.81, 132.86, 137.48, 145.90 and 147.99.

### Example 10

### 4-(1-Methyl-1H-benzimidazol-2-yl)-1-phenylpyridin-1-ium triflate

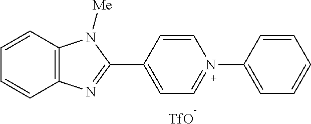

A mixture of 1-methyl-2-(pyridin-4-yl)-1H-benzimidazole (1.00 g, 4.8 mmol), diphenyliodonium triflate (3.08 g, 7.2 mmol), Cu(OAc)₂·H₂O (9.6 mg, 0.48 mmol, 10 mol %) in dry DMF (50 mL) was heated at 100° C. for 16 h, cooled and the solvent removed under reduced pressure. The residue was triturated with Et₂O (100 mL), air dried and then crystallised from hot MeOH (10 mL). The product was isolated by filtration, washed with MeOH and air dried to give the title compound (1.33 g, 64%) as a yellow powder, δ_{H} [(CD₃)₂CO, 400 MHz] 4.294 (3H, s), 7.38-7.45 (1H, m), 7.47-7.53 (1H, m), 7.71-7.90 (5H, m), 8.01-8.11 (2H, m), 8.95 (2H, d, 7 Hz) and 9.51 (2H, d, J=7 Hz); δ_{F} [(CD₃)₂CO, 376 MHz]-78.86; δ_{C} [(CD₃)₂CO, 100 MHz] 32.21, 111.26, 120.77, 123.65, 124.62, 125.32, 127.03, 130.60, 131.68, 138.12, 142.98, 143.39, 144.99, 146.72 and 146.88.

### Compound 10: 1,3-Dimethyl-2-(1-phenylpyridin-1-ium-4-yl)-1H-benzimidazol-3-ium bis(tetrafluoroborate)

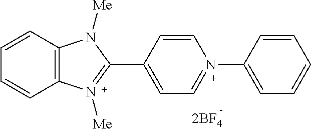

A mixture of 4-(1-methyl-1H-benzimidazol-2-yl)-1-phenylpyridin-1-ium triflate (0.54 g, 1.2 mmol) in MeOTs (4.62 g, 24.8 mmol) was heated at 180° C. for 3 h, cooled, triturated with Et₂O (50 mL), the residue was filtered off, washed with Et₂O (2×10 mL) and air dried. The residue was dissolved in MeOH (10 mL) and added dropwise to a solution of NaBF₄ (2.73 g, 24.8 mmol) in water (50 mL) with stirring. Stirring was continued for 0.5 h and the resulting precipitate filtered, washed with water (2×5 mL) and air dried. The residue was triturated with hot MeOH (5 mL), filtered and air dried to give the title compound (0.49 g, 83%) as a pale tan powder, δ_{H} (DMSO-d₆, 400 MHz) 4.04 (6H, s), 7.80-8.01 (7H, m), 8.19-8.29 (2H, m), 8.81 (2H, d, J=6.5 Hz) and 9.82 (2H, d, J=6.5 Hz); δ_{F} (DMSO-d₆, 376 MHz) −148.26, −148.20; δ_{C} (DMSO-d₆, 100 MHz) 33.49, 114.25, 125.24, 128.15, 130.84, 130.99, 132.41, 132.53, 137.83, 142.96, 145.50 and 146.81.

### Example 11

### Compound 11: 1-Hexyl-3-methyl-2-(1-phenylpyridin-1-ium-4-yl)-1H-benzimidazol-3-ium bis(hexafluorophosphate)

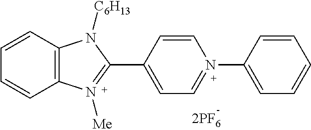

A mixture of 1-hexyl-2-(pyridin-4-yl)-1H-benzimidazole (0.8.0 g, 2.9 mmol), diphenyliodonium triflate (1.85 g, 4.3 mmol), Cu(OAc)₂·H₂O (58 mg, 0.29 mmol, 10 mol %) in dry DMF (30 mL) was heated at 100° C. for 16 h, cooled and the solvent removed under reduced pressure. The residue was triturated with Et₂O (3×30 mL) and dried under vacuum. The residue was heated in MeOTs (4.26 g, 22.4 mmol) at 180° C. for 2 h, cooled and then diluted with Et₂O (80 mL). The solvent was decanted and the residue triturated with Et₂O (3×30 mL) and dried under vacuum. The residue was dissolved in MeOH (20 mL) and added drop wise to a solution of ammonium hexafluorophosphate (4.16 g, 28.7 mmol) in water (100 mL). The precipitate was filtered and washed with water (2×10 mL) and air dried to give the title compound (0.68 g, 38%) as a cream powder, δ_{H} [(CD₃)₂CO, 400 MHz] 0.82 (3H, t, J=6.9 Hz), 1.17-1.45 (6H, m), 1.96-2.04 (2H, m), 4.19 (3H, s), 4.66 (2H, t, J=7.7 Hz), 7.81-7.95 (5H, m), 7.99-8.10 (2H, m), 8.17-8.32 (2H, m), 9.13 (2H, bd, J=5.2 Hz) and 9.89 (2H, bd, J=5.2 Hz); δ_{F} [(CD₃)₂CO, 376 MHz]-72.47 (d, J=710 Hz); δ_{C} [(CD₃)₂CO, 100 MHz] 13.31, 22.18, 25.82, 29.49, 31.06, 32.92, 47.14, 113.76, 113.94, 124.76, 128.08, 130.75, 130.95, 131.89, 132.35, 132.96, 138.59, 143.02, 144.71 and 147.04.

### 3. Imidazo[1,2-a]pyridines

### Example 12

### 7-Bromo-2-phenylimidazo[1,2-a]pyridine A

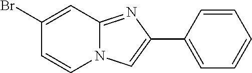 [SMILES: Brc1ccn2cc(-c3ccccc3)nc2c1]

A mixture of phenacyl bromide (4.00 g, 20.1 mmol), 4-bromopyridin-2-amine (2.90 g, 16.8 mmol) and NaHCO₃ (1.69 g, 20.1 mmol) in MeOH (80 mL) was heated at reflux for 5 h, cooled and the solvent reduced. Water (200 mL) was added and the resulting mixture extracted with DCM (3×100 mL), dried (Na₂SO₄) and the solvent removed under reduced pressure. The residue was chromatographed on silica using MeOH (5% in DCM) as eluent. The solvent was removed under reduced pressure and the residue crystallised from DCM/hexanes to give the title compound (2.07 g, 39%) as a cream powder, δ_{H} (CDCl₃, 400 MHz) 6.91 (1H, dd, J=1.9 and 7.1 Hz), 7.34-7.40 (1H, m), 7.43-7.49 (2H, m), 7.83-7.87 (2H, m), 7.92-7.98 (2H, m) and 8.00 (2H, dd, J=0.5 and 7.1 Hz); δ_{C} (CDCl₃, 100 MHz) 108.27, 116.34, 118.21, 119.80, 125.72, 126.12, 128.30, 128.80, 133.27, 145.83 and 146.71.

### 2-Phenyl-7-(pyridin-4-yl)imidazo[1,2-a]pyridine C

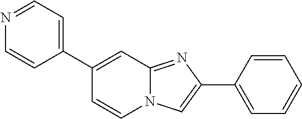 [SMILES: c1ccc(-c2cn3ccc(-c4ccncc4)cc3n2)cc1]

A mixture of 7-bromo-2-phenylimidazo[1,2-a]pyridine A (1.18 g, 4.3 mmol), and 4-(4,4,5,5-tetramethyl-1,3,2-dioxaborolan-2-yl)pyridine (1.32 g, 6.4 mmol), K₂CO₃ (0.90 g, 6.5 mmol) and Pd(PPh₃)₄ (0.20 g, 4 mol %) in degassed PhMe (40 mL) and EtOH (40 mL) under N₂ was heated at reflux for 16 h, cooled, poured into water (50 ml) and extracted with DCM (3×40 mL). The combined extracts were dried (Na₂SO₄) and the solvent removed under reduced pressure. The residue was chromatographed on silica using MeOH (5% in DCM) as eluent. The band with Rf=0.5 was collected, the solvent removed and the residue triturated with hot PhMe (15 mL). After cooling the product was collected and air dried to give the title compound (1.02 g, 87%) as a cream powder, δ_{H} (CDCl₃, 400 MHz) 7.12 (1H, dd, J=1.6 and 7 Hz), 7.35-7.42 (1H, m), 7.45-7.52 (2H, m), 7.59 (2H, dd, J=1.5 and 4.6 Hz), 7.94-8.03 (4H, m), 8.25 (1H, dd, J=0.9 and 7 Hz) and 8.74 (2H, dd, J=1.4 and 4.7 Hz); δ_{C} (CDCl₃, 100 MHz) 108.43, 111.29, 115.22, 120.96, 125.90, 126.12, 128.34, 128.83, 133.42, 134.26, 145.67, 145.87, 147.38 and 150.66.

### 1-Hexyl-4-(2-phenylimidazo[1,2-a]pyridin-7-yl)pyridin-1-ium iodide F

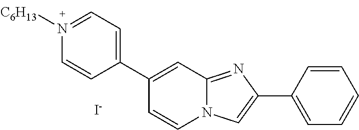

A mixture of 2-phenyl-7-(pyridin-4-yl)imidazo[1,2-a]pyridine C (1.02 g, 3.8 mmol) and 1-iodohexane (2.39 g, 11.3 mmol) in MeCN (40 mL) was heated at reflux for 16 h, cooled, the solvent reduced, and the mixture diluted with Et₂O (50 mL). The precipitate was filtered, washed with Et₂O (2×30 mL) and air dried to give the title compound (1.81 g, 99%) as an orange powder, δ_{H} (CD₃OD, 400 MHz) 0.94 (3H, t, J=6.9 Hz), 1.32-1.51 (6H, m), 2.00-2.14 (2H, m), 4.63 (2H, t, J=7.4 Hz), 7.37-7.57 (4H, m), 7.99 (2H, d, J=7.9 Hz), 8.29 (1H, s), 8.43 (1H, s), 8.53 (2H, d, J=6.3 Hz), 8.68 (1H, d, J=7.1 Hz) and 9.01 (2H, d, J=6.3 Hz); δ_{C} (CD₃OD, 100 MHz) 12.85, 22.08, 25.50, 30.91, 30.98, 60.92, 110.62, 110.81, 116.23, 124.51, 125.89, 127.48, 128.49, 128.61, 130.34, 132.67, 144.53 and 153.97

### 1-Hexyl-4-(2-phenylimidazo[1,2-a]pyridin-7-yl)pyridin-1-ium tetrafluoroborate

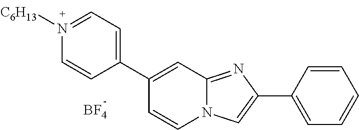

A solution of 1-hexyl-4-(2-phenylimidazo[1,2-a]pyridin-7-yl)pyridin-1-ium iodide F (1.81 g, 3.7 mmol) in MeOH (20 mL) was added dropwise to a solution of NaBF₄ (2.47 g, 22.4 mmol) in water (100 mL) with stirring. Stirring was continued for 0.5 h and the resulting precipitate filtered, washed with water (5 mL) and air dried to give the title compound (1.66 g, 100%) as a golden yellow powder which was used without further purification in the next step.

### Compound 12: 7-(1-Hexylpyridin-1-ium-4-yl)-1-methyl-2-phenylimidazo[1,2-a]pyridin-1-ium bis(tetrafluoroborate)

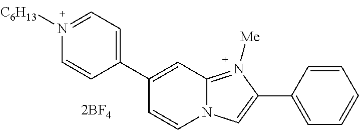

A mixture of 1-hexyl-4-(2-phenylimidazo[1,2-a]pyridin-7-yl)pyridin-1-ium tetrafluoroborate (1.66 g, 3.7 mmol) and MeOTs (2.79 g, 15 mmol) was heated at 180° C. for 2 h, cooled, triturated with Et₂O (3×50 mL) and air dried. The residue was dissolved in MeOH (25 mL) and added dropwise to a solution of NaBF₄ (16.48 g, 150 mmol) with stirring. The resulting precipitate was filtered, washed with water (5 mL), then dissolved in warm MeOH (50 mL) and added dropwise to a solution of NaBF₄ (16.48 g, 150 mmol) in water (250 mL) with stirring. Stirring was continued for 0.5 h and the resulting precipitate filtered. The residue was triturated with EtOH (3 ml), filtered and air dried to give the title compound (0.38 g, 19%) as a pale green powder. The ethanolic liquors were added dropwise to a solution of NaBF₄ (16.48 g, 150 mmol) in water (50 mL) with stirring. The resulting precipitate was filtered, washed with water (10 mL) and air dried to give a second crop (0.45 g, 22%) as a khaki powder; δ_{H} [(CD₃)₂CO, 400 MHz] 0.88 (3H, t, J=6.8 Hz), 1.26-1.55 (6H, m), 2.12-2.27 (2H, m), 4.26 (3H, s), 4.91 (2H, t, J=7.2 Hz), 7.70 (3H, bs), 7.82 (2H, bs), 8.26 (1H, bs, J=6.6 Hz), 8.69 (1H, bs), 8.86 (2H, bd, J=5.8 Hz), 9.05 (1H, bs), 9.21 (1H, bd, J=6.6 Hz) and 9.34 (2H, bd, J=5.8 Hz); δ_{F} (CDCl₃, 376 MHz) −151.02, −150.97; δ_{C} (CDCl₃, 100 MHz) 13.31, 22.16, 25.54, 30.98, 31.26, 32.42, 61.73, 111.65, 113.97, 116.12, 125.24, 126.39, 129.44, 130.01, 130.29, 131.13, 138.32, 140.23, 140.38, 145.62 and 151.68.

### Example 13

### 7-Bromo-2-(pyridin-4-yl)imidazo[1,2-a]pyridine B

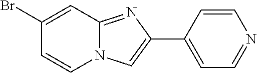 [SMILES: Brc1ccn2cc(-c3ccncc3)nc2c1]

A mixture of 4-(bromoacetyl)pyridine hydrobromide (8.00 g, 28.5 mmol), 4-bromopyridin-2-amine (4.1 g, 23.7 mmol) and NaHCO₃ (4.78 g, 56.9 mmol) in MeOH (80 mL) was heated at reflux for 5 h, cooled and the solvent reduced in volume. Water (200 mL) was added and the resulting mixture extracted with DCM (3×150 mL), dried (Na₂SO₄) and the solvent removed under reduced pressure. The residue was chromatographed on silica using MeOH (5% in DCM) as eluent. The fluorescent band with Rf=0.6 (5% MeOH in DCM) was collected. The solvent was removed under reduced pressure and the residue crystallised from MeOH (5 mL) at 3° C., filtered and air dried to give the title compound (0.68 g, 10%) as a light tan powder, δ_{H} (CDCl₃, 400 MHz) 6.96 (1H, dd, J=1.7 and 7.2 Hz), 7.82 (2H, d, J=6 Hz), 7.86 (1H, bs), 8.00 (1H, bs), 8.04 (1H, d, J=7.2 Hz) and 8.69 (2H, d, J=6 Hz); δ_{C} (CDCl₃, 100 MHz) 110.02, 117.09, 119.21, 120.19, 120.33, 125.95, 140.69, 143.88, 146.07 and 150.40.

### 7-Phenyl-2-(pyridin-4-yl)imidazo[1,2-a]pyridine D

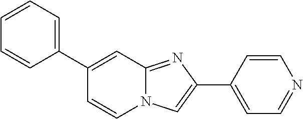 [SMILES: c1ccc(-c2ccn3cc(-c4ccncc4)nc3c2)cc1]

A mixture of 7-bromo-2-(pyridin-4-yl)imidazo[1,2-a]pyridine B (1.17 g, 4.3 mmol) and phenylboronic acid (0.78 g, 6.4 mmol), K₂CO₃ (0.88 g, 6.4 mmol) and Pd(PPh₃)₄ (0.25 g, 5 mol %) in degassed PhMe (30 mL) and EtOH (30 mL) under N₂ was heated at reflux for 24 h, cooled, poured into water (100 mL) and extracted with DCM (3×80 mL). The extracts were dried (Na₂SO₄) and the solvent removed under reduced pressure. The residue was chromatographed on silica using MeOH (5% in DCM) as eluent. The solvent was removed under reduced pressure and the residue triturated with hexanes containing a few drops of DCM. The residue was filtered off, washed with hexanes and air dried to give the title compound (1.02 g, 88%) as a light tan powder, δ_{H} (CDCl₃, 400 MHz) 7.16 (1H, dd, J=1.6 and 7 Hz), 7.41-7.57 (2H, m), 7.66-7.72 (2H, m), 7.83-7.92 (3H, m), 8.03 (1H, s), 8.21 (1H, d, J=7 Hz) and 8.70 (2H, d, J=6 Hz); δ_{C} (CDCl₃, 100 MHz) 109.55, 113.14, 114.54, 120.29, 125.66, 126.74, 128.53, 129.19, 138.40, 138.57, 141.16, 143.81, 146.49 and 150.37.

### 1-Hexyl-4-(7-phenylimidazo[1,2-a]pyridin-2-yl)pyridin-1-ium iodide G

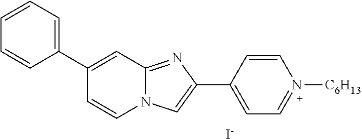

A mixture of 7-phenyl-2-(pyridin-4-yl)imidazo[1,2-a]pyridine D (1.00 g, 3.7 mmol) and 1-iodohexane (2.62 g, 12.4 mmol) in MeCN (40 mL) was heated at reflux for 16 h, cooled, diluted with Et₂O (100 ml) and stirred for 0.5 h. The resulting precipitate was filtered, washed with Et₂O (3×40 mL) and air dried to give the title compound (1.73 g, 97%) as a dull yellow powder, δ_{H} (CD₃OD, 400 MHz) 0.95 (3H, t, J=6.8 Hz), 1.32-1.54 (6H, m), 2.00-2.13 (2H, m), 4.61 (2H, t, J=7.3 Hz), 7.40-7.59 (4H, m), 7.81 (2H, d, J=7.6 Hz), 7.87 (1H, s), 8.56 (2H, d, J=6 Hz), 8.61 (1H, d, J=7.2 Hz), 8.87 (1H, s) and 8.96 (2H, d, J=6 Hz); δ_{C} (CD₃OD, 100 MHz) 12.85, 22.07, 25.49, 30.90, 60.82, 112.96, 114.05, 115.63, 123.02, 126.49, 127.25, 128.74, 128.94, 137.62, 138.98, 140.88, 144.41, 147.30 and 149.56.

### Compound 13: 2-(1-Hexylpyridin-1-ium-4-yl)-1-methyl-7-phenylimidazo[1,2-a]pyridin-1-ium bis(tetrafluoroborate)

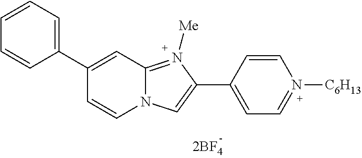

A solution of 1-hexyl-4-(7-phenylimidazo[1,2-a]pyridin-2-yl)pyridin-1-ium iodide G (1.73 g, 3.8 mmol) in MeOH (50 mL) was added dropwise by filtration through a cotton wool plug to NaBF₄ (10.00 g, 90 mmol) water (100 mL) with stirring. Stirring was continued for 0.5 h and the resulting precipitate was collected by filtration, washed with water (2×5 mL) and air dried. The resulting solid and MeOTs (2.87 g, 15.4 mmol) was heated at 180° C. with stirring for 2 h. The resulting oil was cooled, triturated with Et₂O (5×50 mL) and air dried. The resulting gummy solid was dissolved in MeOH (30 mL) and added dropwise to a solution of NaBF₄ (7.54 g, 68.5 mmol) in water (100 mL) with stirring. Stirring was continued for 0.5 h and the resulting precipitate collected, dissolved in hot MeOH (60 mL) and added dropwise to NaBF₄ (7.54 g, 68.5 mmol) in water (100 mL) with stirring. Stirring was continued for 0.5 h and the resulting precipitate filtered, washed with water (2×10 mL) and air dried. The solid was crystallised from hot MeOH (30 mL), filtered and air dried to give the title compound (1.37 g, 74%) as a tan powder, δ_{H} [(CD₃)₂CO, 400 MHz] 0.90 (3H, t, J=7 Hz), 1.29-1.59 (6H, m), 2.16-2.28 (2H, m), 4.41 (3H, s), 4.96 (2H, t, J=7.5 Hz), 7.59-7.69 (3H, m), 8.02-8.16 (3H, m), 8.65 (1H, bs), 8.70 (2H, d, J=6.2 Hz), 9.02 (1H, bs), 9.12 (1H, d, J=7 Hz) and 9.41 (2H, d, J=6.2 Hz); δ_{F} [(CD₃)₂CO, 376 MHz] −151.24, −151.18; δ_{C} [(CD3)₂CO, 100 MHz] 13.29, 22.14, 25.54, 30.98, 31.25, 32.80, 62.15, 107.70, 116.81, 117.43, 127.72, 128.36, 129.55, 130.05, 130.73, 133.24, 135.84, 142.18, 142.50, 145.77 and 147.56.

### Example 14

### 2,7-Di(pyridin-4-yl)imidazo[1,2-a]pyridine E

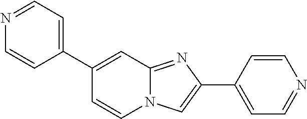 [SMILES: c1cc(-c2ccn3cc(-c4ccncc4)nc3c2)ccn1]

A mixture of 7-bromo-2-(pyridin-4-yl)imidazo[1,2-a]pyridine B (1.25 g, 4.6 mmol) and 4-(4,4,5,5-tetramethyl-1,3,2-dioxaborolan-2-yl)pyridine (1.22 g, 5.9 mmol), K₂CO₃ (0.82 g, 5.9 mmol) and Pd(PPh₃)₄ (0.26 g, 5 mol %) in degassed PhMe (30 mL) and EtOH (30 mL) under N₂ was heated at reflux for 16 h, cooled and the solvent removed under reduced pressure. The residue was washed with water (3×50 mL) and air dried. The residue was chromatographed on silica (pretreated with Et₃N 10% in DCM) using Et₃N (10% in DCM) to Et₃N/MeOH/DCM (12/3/85) gradient as eluent. The fractions were evaporated under reduced pressure and the residue triturated with hexanes. The resulting solid was dissolved in MeOH (10 mL) and water (100 mL) added. The resulting precipitate was filtered, washed with water and air dried to give a yellow powder which was used without further purification in the next step, δ_{H} (CD₃OD, 400 MHz) 7.38 (1H, dd, J=1.7 and 7.2 Hz), 7.84 (2H, d, J=6.3 Hz), 7.96 (2H, d, J=6.2 Hz), 7.98 (1H, bs), 8.51 (1H, bs), 8.54-8.62 (2H, m) and 8.65 (2H, d, J=6.2 Hz); δ_{C} (CD₃OD, 100 MHz) 111.88, 111.99, 114.05, 120.46, 121.38, 127.36, 135.64, 141.74, 142.94, 145.89, 146.28, 149.28 and 149.58.

### 4,4′-(Imidazo[1,2-a]pyridine-2,7-diyl)bis(1-hexylpyridin-1-ium) bis(tetrafluoroborate)

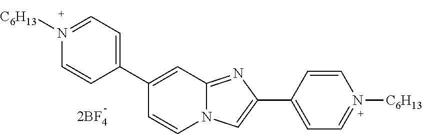

A solution of 2,7-di(pyridin-4-yl)imidazo[1,2-a]pyridine E (0.80 g, 2.9 mmol) and 1-iodohexane (3.74 g, 17.6 mmol) in MeCN (50 mL) was heated at reflux for 16 h, cooled, the solvent reduced in volume (˜20 mL) and Et₂O (60 mL) added. The resulting precipitate was filtered, washed with Et₂O (2×30 mL) and air dried to give 1.69 g. The yellow powder dissolved in MeOH (60 mL) was added dropwise with via filtration through a cotton wool plug to NaBF₄ (6.47 g, 58.8 mmol) in water (300 mL) with stirring. The resulting precipitate was filtered, washed with ice cold water (2×20 mL) and air dried to give the title compound (1.32 g, 72%) as a green fluorescent powder, δ_{H} (DMSO-d₆, 400 MHz) 0.83-0.94 (6H, m), 1.24-1.39 (12H, m), 1.90-2.03 (4H, m), 4.54-4.65 (4H, m), 7.75 (1H, dd, J=1.4 and 7.3 Hz), 8.62-8.76 (5H, m), 8.93 (2H, d, J=7.2 Hz), 9.10 (2H, d, J=6.7 Hz), 9.16 (1H, bs) and 9.20 (2H, d, J=6.7 Hz); δ_{F} (DMSO-d₆, 376 MHz) −148.21, −148.16; δ_{C} (DMSO-d₆, 100 MHz) 14.31, 14.32, 22.34, 22.35, 25.57, 25.59, 31.02, 31.07, 31.10, 60.57, 60.61, 112.43, 117.54, 118.58, 123.59, 125.09, 129.20, 132.08, 141.17, 145.40, 145.51, 145.89, 148.60 and 152.42.

### Compound 14: 4,4′-(1-Methylimidazo[1,2-a]pyridine-1-ium-2,7-diyl)bis(1-hexylpyridin-1-ium) tris(tetrafluoroborate)

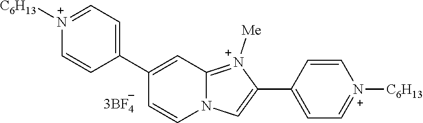

A mixture of 4,4′-(imidazo[1,2-a]pyridine-2,7-diyl)bis(1-hexylpyridin-1-ium) bis(tetrafluoroborate) (1.17 g, 1.9 mmol) and MeOTs (2.83 g, 15.2 mmol) was heated at 180° C. for 2 h, cooled and triturated with Et₂O (50 mL). The residue was filtered and dried under vacuum to give 1.85 g. The solid was dissolved in MeOH (30 mL), added dropwise to a solution of NaBF₄ (30 g, 270 mmol) in water (250 mL) with stirring. Stirring was continued for 0.5 h and the resulting precipitate filtered, washed with water (2×5 mL) and air dried. The residue was dissolved in MeOH/H₂O (30 mL, 1:1) and added dropwise to NaBF₄ (20 g, 182 mmol) in water (150 ml) with stirring. Stirring was continued for 0.5 h and the resulting precipitate filtered, washed with water (2×5 mL) and air dried to give the title compound (0.99 g, 73%) as a grey powder, δ_{H} [(CD₃)₂CO, 400 MHz] 0.84-0.96 (6H, m), 1.27-1.59 (12H, m), 2.16-2.28 (4H, m), 4.42 (3H, s), 4.91-5.04 (4H, m), 8.33 (1H, bd, J=6.7 Hz), 8.73 (2H, bd, J=6 Hz), 8.90 (2H, bd, J=6 Hz), 8.12 (2H, app. bs), 9.29 (1H, bd, J=7 Hz), 8.40 (2H, bd, J=6.4 Hz) and 9.45 (2H, bd, J=6 Hz); δ_{F} [(CD₃)₂CO, 376 MHz]−151.17, −151.12; δ_{C} [(CD3)₂CO, 100 MHz] 13.29, 22.14, 25.54, 30.98, 31.25, 31.28, 33.13, 61.85, 62.27, 111.97, 116.76, 117.63, 126.64, 128.75, 130.89, 134.01, 140.01, 141.69, 141.84, 145.70, 145.85 and 151.48.

### 4. Phenanthroimidazoles

### Example 15

### 6,9-Dibromo-1,2-bis[4-(tert-butyl)phenyl]-1H-phenanthro[9,10-d]imidazole

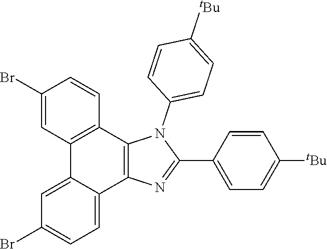 [SMILES: CC(C)(C)c1ccc(-c2nc3c4ccc(Br)cc4c4cc(Br)ccc4c3n2-c2ccc(C(C)(C)C)cc2)cc1]

To a stirred mixture of 4-tert-butylaniline (2.20 g, 14.6 mmol, 2.35 mL, 1.5 equiv.) and 4-tert-butylbenzaldehyde (1.58 g, 9.75 mmol, 1.53 mL) was added acetic acid (100 mL), followed by 3,6-dibromophenanthrene-9,10-dione (3.58 g, 9.75 mmol) and ammonium acetate (9.38 g, 122 mmol, 12.5 equiv.), and the mixture heated at reflux under argon for 2 days. Methanol (20 mL) was added carefully, followed by water until the solution became cloudy. After cooling, the precipitate was collected by vacuum filtration and washed thoroughly with 1:1 water:methanol to give the title compound (6.20 g, 99%) a green powder, δ_{H} (CDCl₃, 400 MHz) 1.29 (9H, s), 1.45 (9H, s), 6.96 (1H, d, J=8.9 Hz), 7.30 (2H, app d, J=8.5 Hz), 7.37 (1H, dd, J=1.9 and 8.9 Hz), 7.40 (2H, app. d, J=8.5 Hz), 7.51 (2H, app. d, J=8.5 Hz), 7.61 (2H, app. d, J=8.5 Hz), 7.83 (1H, dd, J=1.7 and 8.5 Hz) and 8.69-8.76 (3H, m); δ_{C} (CDCl₃, 100 MHz) 31.16, 31.41, 34.70, 35.07, 119.07, 119.84, 122.01, 122.35, 124.59, 125.25, 125.95, 126.17, 126.84, 127.18, 127.22, 128.00, 128.41, 128.63, 128.88, 129.53, 129.89, 130.87, 135.61, 137.06, 151.67, 152.23 and 153.56.

### 1,2-Bis[4-(tert-butyl)phenyl]-6,9-di(pyridin-4-yl)-1H-phenanthro[9,10-d]imidazole

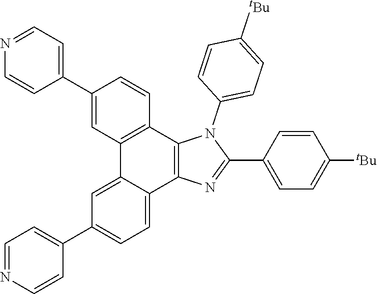 [SMILES: CC(C)(C)c1ccc(-c2nc3c4ccc(-c5ccncc5)cc4c4cc(-c5ccncc5)ccc4c3n2-c2ccc(C(C)(C)C)cc2)cc1]

From 6,9-dibromo-1,2-bis-[4-(tert-butyl)phenyl]-1H-phenanthro[9,10-d]imidazole (5.25 g, 8.20 mmol), 4-(4,4,5,5-tetramethyl-1,3,2-dioxaborolan-2-yl)pyridine (3.53 g, 17.22 mmol, 2.1 equiv.), Pd(PPh₃)₄ (0.34 g, 0.287 mmol, 3.5 mol %), and K₂CO₃ (3.74 g, 17.22 mmol, 2.1 equiv.) in degassed PhMe/EtOH (1:1, 120 mL) for 5 days. After extraction and subsequent removal of the solvent the residue was slurried with DCM and passed through silica, followed by MeOH. The solvent was removed in vacuo, the residue triturated with Et₂O, and the resulting solid collected by vacuum filtration and air dried to give the title compound (4.28 g, 82%) as a dark powder. δ_{H} (CDCl₃, 400 MHz) 1.31 (9H, s), 1.48 (9H, s), 7.27 (1H, d, J=8.5 Hz), 7.32 (2H, d, J=8.5 Hz), 7.47 (2H, d, J=8.4 Hz), 7.55-7.58 (3H, m), 7.64-7.67 (4H, m), 7.75 (2H, d, J=6 Hz), 8.01 (1H, dd, J=1.2 and 8.3 Hz), 8.72 (2H, d, J=6 Hz), 7.76 (2H, d, J=6 Hz), 8.95-9.03 (3H, m); δ_{C} (CDCl₃, 100 MHz) 31.18, 31.45, 34.73, 35.11, 121.74, 121.78, 121.81, 122.02, 122.48, 123.70, 123.95, 125.29, 125.33, 126.32, 127.20, 127.36, 127.92, 128.39, 128.50, 128.57, 128.92, 129.28, 134.30, 135.29, 135.80, 137.70, 148.25, 148.84, 150.41, 150.42, 151.97, 152.27 and 153.55 Compound 15: 4,4′-([1,2-bis(4-(tert-Butyl)phenyl]-3-methyl-1H-phenanthro[9,10-d]imidazole-3-ium-6,9-diyl)bis(1-methylpyridin-1-ium) tris(tetrafluoroborate)

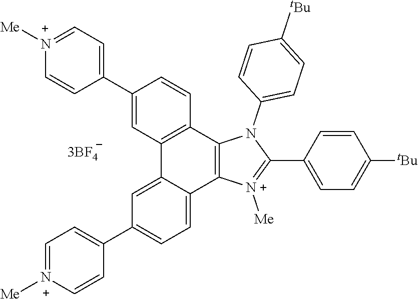 [SMILES: C[n+]1ccc(-c2ccc3c(c2)c2cc(-c4cc[n+](C)cc4)ccc2c2c3n(-c3ccc(C(C)(C)C)cc3)c(-c3ccc(C(C)(C)C)cc3)[n+]2C)cc1]

A mixture of 1,2-bis[4-(tert-butyl)phenyl]-6,9-di(pyridin-4-yl)-1H-phenanthro[9,10-d]imidazole (1.27 g, 2.0 mmol) and MeOTs (3.36 g, 18 mmol, 9.0 equiv.) was heated to 180° C. whilst stirring under argon for 8 hours, cooled, triturated with Et₂O, and the solid collected by vacuum filtration to give a grey powder (2.75 g). A mixture of the crude product (1.17 g) and MeOTs (3.6 g) was heated to 180° C. whilst stirring under argon for 32 hours, cooled, triturated with Et₂O, and the solid collected by vacuum filtration. A filtered solution of the solid in hot 1:1 MeOH:water was added dropwise whilst stirring to a solution of NaBF₄ (2.64 g, 24 mmol, 12.0 equiv) in water (25 mL) to give the title compound (0.71 g, 36%) as a green powder, δ_{H} (DMSO-d₆, 400 MHz) 1.28 (9H, s), 1.35 (9H, s), 4.39 (3H, s), 4.45 (6H, bs), 7.19 (1H, d, J=8.8 Hz), 7.59-7.77 (9H, m), 8.26 (1H, dd, J=1.5 and 8.9 Hz), 8.65 (1H, dd, J=1.5 and 8.9 Hz), 8.85 (2H, app. d, J=7 Hz), 8.95 (2H, app. d, J=6.7 Hz), 9.10-9.22 (5H, m), 9.88 (1H, d, J=1.5 Hz) and 9.93 (1H, d, J=1.6 Hz). δ_{F} (DMSO-d₆, 376 MHz) −148.26 and −148.21; δ_{C} (DMSO-d₆, 100 MHz) 31.17, 31.39, 35.40, 38.42, 47.76, 47.83, 118.83, 122.65, 122.76, 123.48, 124.70, 125.48, 125.64, 125.78, 126.23, 126.36, 127.19, 127.65, 127.90, 128.27, 128.42, 128.53, 130.25, 130.36, 131.57, 132.61, 133.41, 133.51, 138.02, 146.12, 146.25, 151.71, 153.58, 153.72, 155.16 and 155.90.

### Example 16

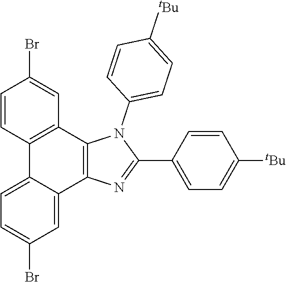 [SMILES: CC(C)(C)c1ccc(-c2nc3c4cc(Br)ccc4c4ccc(Br)cc4c3n2-c2ccc(C(C)(C)C)cc2)cc1]

To a stirred mixture of 4-tert-butylaniline (2.20 g, 14.6 mmol, 2.35 mL, 1.5 equiv.) and 4-tert-butylbenzaldehyde (1.58 g, 9.75 mmol, 1.53 mL) was added acetic acid (100 mL), followed by 2,7-dibromophenanthrene-9,10-dione (3.58 g, 9.75 mmol) and ammonium acetate (9.38 g, 122 mmol, 12.5 equiv.), and the mixed heated under reflux under argon for 2 days. Methanol (20 mL) was added carefully, followed by water until the solution became cloudy. After cooling, the precipitate was collected by vacuum filtration and washed with 1:1 water:methanol to give a tan powder. The crude material was purified by column chromatography (neat DCM) to give the title compound (2.74 g, 44%) an off-white powder, δ_{H} (CDCl₃, 400 MHz) 1.30 (9H, s), 1.47 (9H, s), 6.91 (1H, d, J=1.9 Hz), 7.33 (2H, d, J=8.5 Hz), 7.41 (2H, d, J=8.3 Hz), 7.52 (1H, dd, J=1.9 and 8.7 Hz), 7.62 (2H, d, J=8.5 Hz), 7.66 (2H, d, J=8.3 Hz), 7.69 (1H, dd, J=2 and 9 Hz), 8.42 (1H, d, J=8.7 Hz), 8.47 (1H, d, J=9 Hz) and 9.00 (1H, d, J=2 Hz); δ_{C} (CDCl₃, 100 MHz) 31.17, 31.43, 34.73, 35.12, 120.83, 122.00, 123.76, 124.26, 124.76, 125.36, 125.41, 125.35, 127.05, 127.16, 127.22, 127.34, 127.76, 127.92, 128.38, 128.58, 128.79, 128.84, 135.41, 137.02, 151.40, 152.31 and 153.90.

### 1,2-Bis[4-(tert-butyl)phenyl]-5,10-di(pyridin-4-yl)-1H-phenanthro[9,10-d]imidazole

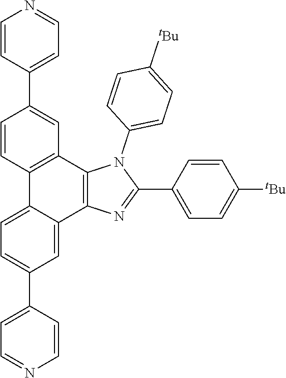 [SMILES: CC(C)(C)c1ccc(-c2nc3c4cc(-c5ccncc5)ccc4c4ccc(-c5ccncc5)cc4c3n2-c2ccc(C(C)(C)C)cc2)cc1]

From 5,10-dibromo-1,2-bis[4-(tert-butyl)phenyl]-1H-phenanthro[9,10-d]imidazole (2.63 g, 4.10 mmol), 4-(4,4,5,5-tetramethyl-1,3,2-dioxaborolan-2-yl)pyridine (1.77 g, 8.61 mmol, 2.1 equiv.), Pd(PPh₃)₄ (0.17 g, 0.14 mmol, 3.5 mol %), and K₂CO₃ (1.87 g, 8.61 mmol, 2.1 equiv.) in degassed PhMe/EtOH (1:1, 80 mL) for 5 days. After extraction and subsequent removal of solvent, the solid was crystallised from hot PhMe to give the title compound (1.33 g, 51%) as a green powder, δ_{H} (CDCl₃, 400 MHz) 1.31 (9H, s), 1.49 (9H, s), 7.22 (2H, dd, J=1.5, 4.6 Hz), 7.35 (2H, app. d, J=8.5 Hz), 7.50-7.56 (3H, m), 7.60 (2H, app. d, J=8.5 Hz), 7.69 (2H, app. d, J=8.8.5 Hz), 7.80-7.86 (3H, m), 7.95 (1H, dd, J=2, 8.6 Hz), 8.57 (2H, dd, J=1.4, 4.6 Hz), 8.76 (2H, dd, J=1.4, 4.6 Hz), 8.81 (1H, d, J=8.8 Hz), 8.86 (1H, d, J=8.8 Hz), 9.21 (1H, d, J=1.9 Hz); δ_{C} (CDCl₃, 100 MHz) 31.17, 31.51, 34.74, 35.13, 119.32, 121.08, 121.22, 121.94, 123.00, 123.78, 124.08, 124.33, 125.17, 125.38, 127.27, 127.32, 128.00, 128.10, 128.24, 128.60, 128.94, 129.05, 135.25, 135.98, 136.95, 137.81, 147.45, 147.93, 150.27, 150.38, 151.60, 152.30 and 153.66.

### Compound 16: 4,4′-([1,2-Bis(4-(tert-butyl)phenyl]-3-methyl-1H-phenanthro[9,10-d]imidazole-3-ium-5,10-diyl)bis(1-methylpyridin-1-ium) tris(tetrafluoroborate)

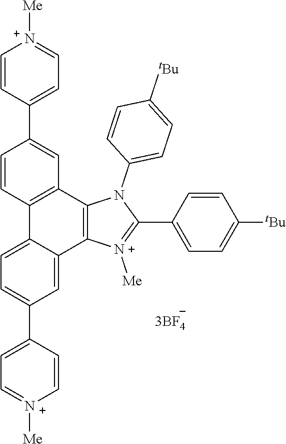 [SMILES: C[n+]1ccc(-c2ccc3c4ccc(-c5cc[n+](C)cc5)cc4c4c(c3c2)n(-c2ccc(C(C)(C)C)cc2)c(-c2ccc(C(C)(C)C)cc2)[n+]4C)cc1]

A mixture of 1,2-bis[4-(tert-butyl)phenyl]-5,10-di(pyridin-4-yl)-1H-phenanthro[9,10-d]imidazole (0.50 g, 0.79 mmol) and MeOTs (2.21 g, 11.9 mmol, 15 equiv.) was heated to 180° C. whilst stirring under argon for 24 hours, cooled, triturated with Et₂O, and the solid collected by vacuum filtration. A filtered solution of the solid in hot 4:1 MeOH:H₂O was slowly added to a stirred solution of NaBF₄ (0.78 g, 7.11 mmol, 9.0 equiv.) in water (15 mL) and stirred for 30 minutes. The precipitate was collected by vacuum filtration and washed with water to give the title compound as a green powder (0.65 g, 86%) as a green powder, δ_{H} (DMSO-d₆, 400 MHz) 1.27 (9H, s), 1.32 (9H, s), 4.35 (3H, s), 4.43 (3H, s), 4.57 (3H, s), 7.56 (1H, d, J=1.7 Hz), 7.63 (2H, d, J=8.5 Hz), 8.69-7.82 (6H, m), 7.98 (2H, d, J=6.8 Hz), 8.44 (1H, dd, J=1.7 and 8.8 Hz), 8.60 (1H, dd, J=1.3 and 8.8 Hz), 8.88 (2H, d, J=6.8 Hz), 8.96 (2H, d, J=6.8 Hz), 9.17 (2H, d, J=6.8 Hz), 9.28 (1H, d, J=1.2 Hz), 9.47-9.57 (2H, m); δ_{F} (DMSO-d₆, 376 MHz) −148.25 and −148.2; δ_{C} (DMSO-d₆, 100 MHz) 31.16, 31.52, 35.40, 35.41, 38.06, 47.83, 47.89, 118.85, 121.13, 121.70, 122.46, 123.15, 124.81, 125.76, 125.90, 126.38, 126.92, 127.30, 127.65, 127.80, 127.87, 127.95, 128.38, 128.67, 131.03, 131.32, 131.61, 132.63, 134.18, 134.99, 146.26, 151.19, 153.50, 153.72, 155.16 and 155.89.

### Example 17

### 6,9-Dibromo-1-[4-(tert-butyl)phenyl]-2-(pyridin-4-yl)-1H-phenanthro[9,10-d]imidazole

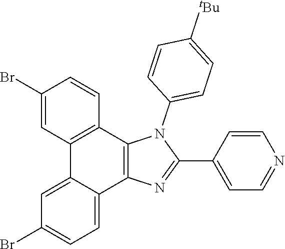 [SMILES: CC(C)(C)c1ccc(-n2c(-c3ccncc3)nc3c4ccc(Br)cc4c4cc(Br)ccc4c32)cc1]

To a stirred mixture of 4-pyridinecarboxaldehyde (1.04 g, 9.75 mmol, 0.91 mL) and 4-tert-butylaniline (2.20 g, 14.6 mmol, 2.35 mL, 1.5 equiv.) was added acetic acid (100 mL), followed by 3,6-dibromophenanthrene-9,10-dione (3.58 g, 9.75 mmol) and ammonium acetate (9.38 g, 122 mmol, 12.5 equiv.), and the mixture heated under reflux under argon for 3 days. Methanol (20 mL) was added carefully, followed by water until the solution became cloudy. After cooling, the precipitate was collected by vacuum filtration and washed with 1 M K₂CO₃ solution, and air dried to give the title compound (4.76 g, 83%) as a green powder, δ_{H} (CDCl₃, 400 MHz) 1.47 (9H, s), 6.99 (1H, d, J=8.9 Hz), 7.37-7.56 (5H, m), 7.66 (2H, d, J=8.5 Hz), 7.84 (1H, dd, J=1.8 and 8.5 Hz), 8.54 (2H, d, J=6.2 Hz) and 8.65-8.75 (3H, m); δ_{C} (CDCl₃, 100 MHz) 154.42, 149.94, 148.27, 137.89, 137.36, 134.98, 131.13, 130.17, 129.05, 128.82, 128.24, 128.10, 127.59, 126.99, 126.06, 125.31, 124.51, 122.74, 122.47, 121.71, 120.39, 119.91, 35.19 and 31.40.

### 1-[4-(tert-Butyl)phenyl]-2,6,9-tri(pyridin-4-yl)-1H-phenanthro[9,10-d]imidazole

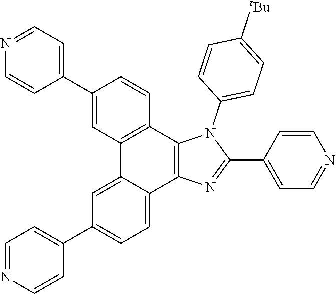 [SMILES: CC(C)(C)c1ccc(-n2c(-c3ccncc3)nc3c4ccc(-c5ccncc5)cc4c4cc(-c5ccncc5)ccc4c32)cc1]

From 6,9-dibromo-1-(4-(tert-butyl)phenyl)-2-(pyridin-4-yl)-1H-phenanthro[9,10-d]imidazole (2.93 g, 5.00 mmol), 4-(4,4,5,5-tetramethyl-1,3,2-dioxaborolan-2-yl)pyridine (2.15 g, 10.5 mmol, 2.1 equiv.), Pd(PPh₃)₄ (0.20 g, 0.18 mmol, 3.5 mol %), and K₂CO₃ (1.45 g, 10.5 mmol, 2.1 equiv.) in degassed PhMe/EtOH (1:1, 120 mL) for 6 days. After extraction and subsequent removal of solvent, the residue was triturated with water (250 mL), then triturated with Et₂O (50 mL). The resulting solid was dissolved in hot 10:1 PhMe:EtOH, cooled to −20° C. overnight, then concentrated in vacuo. The resulting precipitate was collected by vacuum filtration and washed with Et₂O to give the title compound (1.50 g, 52%) as a tan powder, δ_{H} (CDCl₃, 400 MHz) 1.50 (9H, s), 7.31 (1H, d, J=8.7 Hz), 7.48-7.51 (4H, m), 7.56-7.77 (7H, m), 8.04 (1H, dd, J=1.2 and 8.4 Hz), 8.56 (2H, d, J=6.1 Hz), 8.73 (2H, d, 6 Hz), 8.76 (2H, d, J=6 Hz), 8.94-9.05 (3H, m); δ_{C} (CDCl₃, 100 MHz) 31.43, 35.22, 121.81, 121.85, 122.01, 122.03, 122.60, 122.82, 123.38, 123.91, 125.62, 126.62, 127.61, 127.67, 128.19, 128.64, 129.29, 129.82, 135.12, 135.90, 137.63, 137.92, 148.05, 148.61, 149.95, 150.44, 150.47 and 154.42.

### Compound 17: 4,4′,4″-{[1-(4-(tert-Butyl)phenyl]-3-methyl-1H-phenanthro[9,10-d]imidazole-3-ium-2,6,9-triyl}tris(1-methylpyridin-1-ium) tetrakis(tetrafluoroborate)

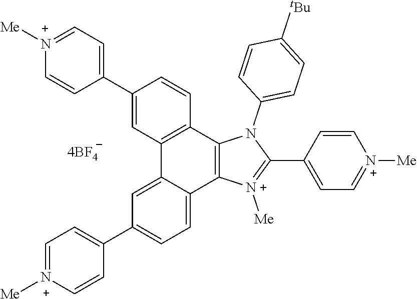 [SMILES: C[n+]1ccc(-c2ccc3c(c2)c2cc(-c4cc[n+](C)cc4)ccc2c2c3n(-c3ccc(C(C)(C)C)cc3)c(-c3cc[n+](C)cc3)[n+]2C)cc1]

A mixture of 1-[4-(tert-butyl)phenyl]-2,6,9-tri(pyridin-4-yl)-1H-phenanthro[9,10-d]imidazole (1.17 g, 2.0 mmol) and MeOTs (2.60 g, 14 mmol, 7.0 equiv.) was heated to 180° C. whilst stirring under argon for 16 hours, cooled, triturated with Et₂O, and the solid collected by vacuum filtration. The solid was ground with a pestle and mortar along with a small amount of Et₂O, triturated with Et₂O, and air drive to give a green-yellow powder. A mixture of the powder (1.55 g) and MeOTs (1.0 g, 5.4 mmol) was heated to 180° C. whilst stirring under argon for 16 hours, cooled, triturated with Et₂O, and the solid collected by vacuum filtration. A filtered solution of the solid in hot MeOH:water (1:1) was added dropwise to a stirred solution of NaBF₄ (2.64 g, 24.0 mmol, 12.0 equiv.) in water (25 mL), stirred for 30 minutes, and the precipitate collected by vacuum filtration to give the title compound (0.71 g, 36%) as a green powder, δ_{H} (DMSO-d₆, 400 MHz) 1.38 (9H, s), 4.39 (3H, s), 4.43 (3H, s), 4.45 (3H, s), 4.51 (3H, s), 7.02 (1H, d, J=8.8 Hz), 8.26 (1H, dd, J=1.8 and 8.8 Hz), 8.50 (2H, d, J=6.8 Hz), 8.65 (1H, dd, J=1.8 and 8.8 Hz), 8.84 (2H, d, J=7.1 Hz), 8.95 (2H, d, J=7 Hz), 9.12-9.22 (5H, m), 9.27 (2H, d, J=7 Hz), 9.87 (1H, d, J=1.8 Hz) and 9.93 (1H, d, J=1.8 Hz); δ_{F} (DMSO-d₆, 376 MHz) −148.27, −148.22; δ_{C} (DMSO-d₆, 400 MHz) 31.41, 35.53, 38.62, 47.81, 47.88, 49.35, 122.24, 122.59, 122.95, 124.97, 125.58, 125.77, 126.37, 127.91, 128.22, 128.36, 128.55, 128.75, 130.62, 130.71, 131.28, 134.02, 134.15, 137.00, 145.64, 146.16, 146.28, 147.59, 153.41, 153.58 and 156.09.

### Example 18

### 6,9-Dibromo-1-[4-(tert-butyl)phenyl]-2-(pyridin-4-yl)-1H-phenanthro[9,10-d]imidazole

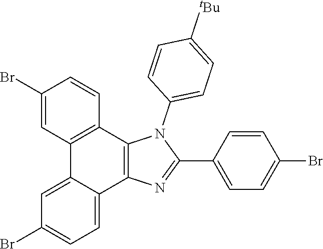 [SMILES: CC(C)(C)c1ccc(-n2c(-c3ccc(Br)cc3)nc3c4ccc(Br)cc4c4cc(Br)ccc4c32)cc1]

To a stirred mixture of 4-tert-butylaniline (2.20 g, 14.6 mmol, 2.35 mL, 1.5 equiv.) and 4-bromobenzaldehyde (1.80 g, 9.75 mmol) was added acetic acid (100 mL), followed by 3,6-dibromophenanthrene-9,10-dione (3.58 g, 9.75 mmol) and ammonium acetate (9.38 g, 122 mmol, 12.5 equiv.), and the mixture heated under reflux under argon for 2 days. Methanol (20 mL) was added carefully, followed by water until the solution became cloudy. After cooling, the precipitate was collected by vacuum filtration and washed with 1:1 water:methanol to give the title compound (6.10 g, 94%) as a green powder, δ_{H} (CDCl₃, 400 MHz) 1.45 (9H, s), 6.97 (1H, d, J=8.9 Hz), 7.34-7.44 (7H, m), 7.61 (2H, d, J=8.4 Hz), 7.82 (1H, dd, J=1.5 and 8.5 Hz) and 8.64-8.74 (3H, m); δ_{C} (CDCl₃, 100 MHz) 31.40, 35.12, 119.40, 120.05, 121.83, 122.34, 123.61, 124.48, 126.00, 126.02, 126.90, 127.37, 128.18, 128.25, 128.68, 129.12, 129.66, 130.01, 130.67, 130.96, 131.49, 135.25, 137.11, 150.33 and 153.93.

### 1-[4-(tert-Butyl)phenyl]-6,9-di(pyridin-4-yl)-2-[4-(pyridin-4-yl)phenyl]-1H-phenanthro[9,10-d]imidazole

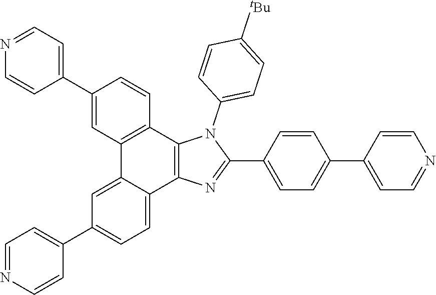 [SMILES: CC(C)(C)c1ccc(-n2c(-c3ccc(-c4ccncc4)cc3)nc3c4ccc(-c5ccncc5)cc4c4cc(-c5ccncc5)ccc4c32)cc1]

From 6,9-dibromo-1-[4-(tert-butyl)phenyl]-2-(4-bromophenyl)-1H-phenanthro[9,10-d]imidazole (5.44 g, 8.20 mmol), 4-(4,4,5,5-tetramethyl-1,3,2-dioxaborolan-2-yl)pyridine (5.30 g, 25.8 mmol, 3.15 equiv.), Pd(PPh₃)₄ (0.34 g, 0.38 mmol, 3.5 mol %), and K₂CO₃ (5.61 g, 25.8 mmol, 3.15 equiv.) in degassed PhMe/EtOH (1:1, 120 mL) for 7 days. After extraction and subsequent removal of solvent, the residue was dissolved in hot 1:1 DCM:isopropanol, cooled to −20° C. overnight, then concentrated in vacuo. The resulting precipitate was collected by vacuum filtration and washed with Et₂O to give the title compound (3.73 g, 59%) as a green powder, δ_{H} (CDCl₃, 400 MHz) 1.49 (9H, s), 7.32 (1H, d, J=8.6 Hz), 7.48-7.53 (4H, m), 7.57-7.62 (3H, m), 7.64-7.71 (4H, m), 7.73-7.79 (4H, m), 8.06 (1H, dd, J=1.4, 8.3 Hz), 8.67 (2H, dd, J=1.5, 4.6 Hz), 8.73 (2H, dd, J=1.5, 4.6 Hz), 8.77 (2H, dd, J=1.5, 4.6 Hz), 8.98-9.07 (3H, m); δ_{C} (CDCl₃, 100 MHz) 31.45, 35.17, 121.46, 121.81, 121.91, 122.05, 122.58, 123.57, 123.93, 125.50, 126.50, 126.84, 127.40, 127.81, 128.23, 128.42, 128.53, 128.87, 129.53, 129.90, 130.98, 134.71, 135.58, 135.62, 137.86, 138.44, 147.30, 148.18, 148.77, 150.38, 150.44, 150.92 and 153.92.

### Compound 18: 4,4′-{1-[4-(tert-Butyl)phenyl]-3-methyl-2-(4-(1-methylpyridin-1-ium-4-yl)phenyl)-1H-phenanthro[9,10-d]imidazole-3-ium-6,9-diyl}bis(1-methylpyridin-1-ium) tetrakis(tetrafluoroborate)

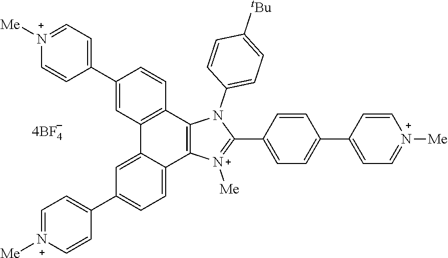 [SMILES: C[n+]1ccc(-c2ccc(-c3n(-c4ccc(C(C)(C)C)cc4)c4c5ccc(-c6cc[n+](C)cc6)cc5c5cc(-c6cc[n+](C)cc6)ccc5c4[n+]3C)cc2)cc1]

A mixture of 1-[4-(tert-butyl)phenyl]-6,9-di(pyridin-4-yl)-2-[4-(pyridin-4-yl)phenyl]-1H-phenanthro[9,10-d]imidazole (3.29 g, 5.0 mmol) and MeOTs (5.59 g, 30 mmol, 6.0 equiv.) was heated to 180° C. whilst stirring under argon for 16 hours, cooled, triturated with Et₂O, and the solid collected by vacuum filtration. The solid was ground with a pestle and mortar along with a small amount of Et₂O, triturated with Et₂O, and air drive to give a green powder. A mixture of the powder (4.91 g) and MeOTs (4.00 g, 21.5 mmol) was heated to 180° C. whilst stirring under argon for 16 hours, cooled, triturated with Et₂O, and the solid collected by vacuum filtration. A filtered solution of the solid in hot 1:1: MeOH:water was added dropwise to a stirred solution of NaBF₄ (6.59 g, 50 mmol, 12.0 equiv.) in water (65 mL), stirred for 30 minutes, and the precipitate collected by vacuum filtration to give the title compound (4.17 g, 78%) as a khaki-green powder, δ_{H} (DMSO-d₆, 400 MHz) 1.35 (9H, s), 4.36 (3H, s), 4.39 (3H, s), 4.44 (3H, s), 4.48 (3H, s), 7.09 (1H, d, J=8.8 Hz), 7.75 (2H, d, 8.6 Hz), 7.80 (2H, d, J=8.6 Hz), 8.08 (2H, d, J=8.4 Hz), 8.25 (1H, dd, J=1.7, 8.9 Hz), 8.32 (2H, d, J=8.5 Hz), 8.56 (2H, d, J=6.9 Hz), 8.65 (1H, dd, J=1.7, 8.8 Hz), 8.85 (2H, d, J=7.1 Hz), 8.95 (2H, d, J=7.1, Ar—H), 9.05-9.27 (7H, m), 9.88 (1H, d, J=1.7 Hz) and 9.95 (1H, d, J=1.7); δ_{F} (DMSO-d₆, 376 MHz) −148.25 and −148.20; δ_{C} (DMSO-d₆, 100 MHz) 31.41, 35.45, 38.48, 47.78, 47.84, 122.63, 123.35, 124.77, 125.21, 125.51, 125.67, 125.78, 126.30, 127.41, 127.88, 128.17, 128.43, 128.60, 129.09, 130.35, 130.48, 132.33, 133.04, 133.57, 133.69, 137.67, 147.13, 146.27, 146.43, 150.26, 152.92, 153.51, 153.67 and 155.48.

### 5. Benzoselenazoles

### Example 19

### 4-(Benzoselenazol-2-yl)-1-hexylpyridin-1-ium iodide

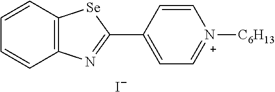

2-(Pyridin-4-yl)benzoselenazole (0.75 g, 2.89 mmol) and 1-iodohexane (1.23 mL, 8.67 mmol) were suspended in MeCN (40 mL) and then stirred at 80° C. for 16 h. The reaction mixture was cooled to ambient temperature and the solvent was removed under reduced pressure. The resulting solid was triturated with acetone (20 mL), filtered, washed with acetone (20 mL) and dried under reduced pressure to give the desired product as an orange powder. Yield 1.10 g, 81%. δ_{H} (CD₃OD, 300 MHz) 9.11 (d, J=6.9 Hz, 2H), 8.68 (d, J=6.9 Hz, 2H), 8.28 (d, J=8.2 Hz, 1H), 8.23 (d, J=8.2 Hz, 1H), 7.65 (dd, J=7.3, 1.2 Hz, 1H), 7.53 (dd, J=7.3, 1.2 Hz, 1H), 4.67 (t, J=7.6, 2H), 1.95 (quint., J=7.4 Hz, 2H), 1.55-1.23 (m, 6H), 0.94 (t, J=6.8 Hz, 3H); δ_{C} (DMSO-d₆, 100 MHz) 166.72, 156.98, 151.53, 146.78, 142.04, 128.82, 128.80, 127.52, 126.90, 126.53, 62.87, 32.41, 32.32, 26.91, 23.48, 14.28.

### Compound 19: 4-(Benzoselenazol-2-yl)-1-hexylpyridin-1-ium tetrafluoroborate

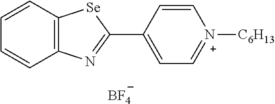

4-(Benzoselenazol-2-yl)-1-hexylpyridin-1-ium iodide (0.65 g, 1.38 mmol) was dissolved in hot MeOH (50 mL) and added dropwise through a cotton wool plug to a stirred solution of NaBF₄ (5.0 g) in H₂O (200 mL) whereupon a pale yellow precipitate formed. The solution was stirred for 30 minutes and then filtered under reduced pressure. The resulting solid was washed with water (30 mL) and then dried under reduced pressure to give the desired product as a pale yellow powder. Yield 0.59 g, 76%. δ_{H} (DMSO-d₆, 400 MHz) 9.20 (d, J=6.7 Hz, 2H), 8.74 (d, J=6.7 Hz, 2H), 8.39 (d, J=8.0 Hz, 1H), 8.28 (d, J=8.0 Hz, 1H), 7.66 (dd, J=7.3, 0.9 Hz, 1H), 7.55 (dd, J=7.3, 0.9 Hz, 1H), 4.64 (t, J=7.4, 2H), 1.95 (quint., J=6.5 Hz, 2H), 1.43-1.20 (m, 6H), 0.87 (t, J=6.7 Hz, 3H); δ_{C} (DMSO-d₆, 100 MHz) 166.57, 154.99, 148.64, 145.71, 140.40, 127.61, 127.34, 126.49, 125.91, 125.21, 60.57, 30.68, 30.56, 25.05, 21.84, 13.81; ⁶B (DMSO-d₆, 128 MHz) −1.3; δ_{F} (DMSO-d₆, 376 MHz) −148.27 (br. m, 4F).

### Example 20

### 4-(Benzoselenazol-2-yl)-1-phenylpyridin-1-ium trifluoromethanesulfonate

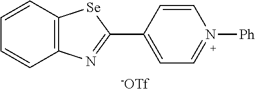

2-(Pyridin-4-yl)benzoselenazole (1.00 g, 3.86 mmol), diphenyliodonium triflate (2.49 g, 5.79 mmol) and Cu(OAc)₂. H₂O (116 mg, 0.58 mmol) were dissolved in DMF (30 mL) under N₂ and the reaction mixture was stirred at 100° C. for 16 h. The reaction mixture was cooled to room temperature and the solvent was removed under reduced pressure. The resulting yellow solid was triturated in hot MeOH, filtered and dried under reduced pressure to give the desired product as a yellow powder. Yield 1.70 g, 91%. δ_{H} (DMSO-d₆, 400 MHz) 9.46 (d, J=6.9 Hz, 2H), 8.86 (d, J=6.9 Hz, 2H), 8.42 (d, J=7.8 Hz, 1H), 8.33 (d, J=7.8 Hz, 1H), 8.00-7.90 (m, 2H), 7.84-7.74 (m, 3H), 7.69 (dd, J=7.4, 1.1 Hz, 1H), 7.58 (dd, J=7.4, 1.1 Hz, 1H); δ_{C} (DMSO-d₆, 100 MHz) 166.28, 155.14, 149.35, 145.83, 142.39, 140.80, 131.40, 130.24, 127.75, 127.55, 126.60, 126.14, 124.97, 124.68, 120.65 (d, J_{C-F}=320.4 Hz); δ_{F} (DMSO-d₆, 376 MHz) −77.76 (s, 3F).

### Compound 20: 4-(Benzoselenazol-2-yl)-1-phenylpyridin-1-ium tetrafluoroborate

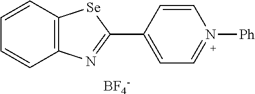

4-(Benzoselenazol-2-yl)-1-phenylpyridin-1-ium trifluoromethanesulfonate (1.00 g, 2.06 mmol) was dissolved in hot MeOH (50 mL) and added dropwise through a cotton wool plug to a stirred solution of NaBF₄ (5.0 g) in H₂O (200 mL) whereupon a yellow precipitate formed. The solution was stirred for 30 minutes and then filtered under reduced pressure. The resulting solid was washed with water (30 mL) and then dried under reduced pressure to give the desired product as a yellow powder. Yield 0.61 g, 70%. δ_{H} (DMSO-d₆, 400 MHz) 9.45 (d, J=6.9 Hz, 2H), 8.55 (d, J=6.9 Hz, 2H), 8.41 (d, J=7.8 Hz, 1H), 7.32 (d, J=7.8 Hz, 1H), 8.00-7.87 (m, 2H), 7.83-7.72 (m, 3H), 7.67 (dd, J=7.4, 1.1 Hz, 1H), 7.57 (dd, J=7.4, 1.1 Hz, 1H); δ_{C} (DMSO-d₆, 100 MHz) 166.30, 155.16, 149.36, 145.84, 142.40, 140.82, 131.43, 130.27, 127.77, 127.57, 126.61, 126.16, 124.99, 124.69; ⁶b (DMSO-d₆, 128 MHz) −1.3; S_{F} (DMSO-d₆, 376 MHz) −148.21 (br. m, 4F).

### Example 21

### Compound 21: 4-(Benzoselenazol-2-yl)-1-phenylpyridin-1-ium hexafluorophosphate

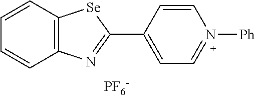

4-(Benzoselenazol-2-yl)-1-phenylpyridin-1-ium trifluoromethanesulfonate (0.20 g, 0.47 mmol) was dissolved in hot MeOH (20 mL) and added dropwise through a cotton wool plug to a stirred solution of NH₄PF₆ (0.50 g) in H₂O (100 mL) whereupon a yellow precipitate formed. The solution was stirred for 30 minutes and then filtered under reduced pressure. The resulting solid was washed with water (30 mL) and then dried under reduced pressure to give the desired product as a yellow powder. Yield 0.13 g, 71%. δ_{H} [(CD₃)₂CO, 400 MHz] 9.52 (d, J=6.3 Hz, 2H), 8.98 (d, J=6.3 Hz, 2H), 8.38 (d, J=8.5 Hz, 1H), 8.36 (d, J=8.5 Hz, 1H), 8.13-7.95 (m, 2H), 7.92-7.78 (m, 3H), 7.73 (t, J=7.6 Hz, 1H), 7.61 (t, J=7.6 Hz, 1H); δ_{C} [(CD₃)₂CO, 100 MHz] 166.22, 156.65, 151.54, 146.67, 143.78, 141.91, 132.64, 131.45, 128.74, 128.71, 127.39, 126.99, 126.34, 125.43; δ_{P} [(CD₃)₂CO, 162 MHz] −144.27 (sept., J=707.8 Hz, 1P); δ_{F} [(CD₃)₂CO, 376 MHz] −72.51 (d, J=707.8 Hz, 6F).

### 6. Benzoxazoles and Benzoisoxazoles

### Example 22

### 2-(Pyridin-4-yl)-2,3-dihydrobenzoxazole

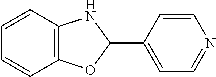 [SMILES: c1ccc2c(c1)NC(c1ccncc1)O2]

A solution of 2-aminophenol (8.00 g, 73.4 mmol) and pyridine-4-carboxaldehyde (7.85 g, 73.4 mmol) in EtOH (350 mL) was stirred under air for 5 days. The solvent was reduced in volume and the resulting solid filtered off, washed with EtOH (20 mL) and air dried to give the title compound (13.36 g, 92%) as an orange powder, δ_{H} (CDCl₃, 400 MHz) 6.93 (1H, app. t, J=7.7 Hz), 7.04 (1H, app. d, J=8.1 Hz), 7.22-7.31 (2H, m), 7.35 (1H, app. d, J=8.0 Hz), 7.76 (2H, bd, J=4.5 Hz), 8.69 (1H, s) and 8.78 (2H, bs).

### 2-(Pyridin-4-yl)benzoxazole

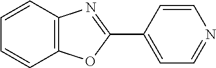 [SMILES: c1ccc2oc(-c3ccncc3)nc2c1]

2,3-Dichloro-5,6-dicyano-1,4-benzoquinone (1.15 g, 5 mmol) was added in one portion to a solution of 2-(pyridin-4-yl)-2,3-dihydrobenzoxazole (1.00 g, 5 mmol) in DCM (50 mL) with stirring. Stirring was continued for 1 h and Et₃N (5 mL) was added. The resulting solution was filtered through silica using DCM (100-0% in EtOAc) as eluent. The first band was collected to give the title compound (0.43 g, 43%) as a pale yellow powder. The second band was collected to give unreacted starting material (0.28 g, 28%), δ_{H} (CDCl₃, 400 MHz) 7.39-7.47 (2H, m), 7.61-7.66 (1H, m), 7.81-7.85 (1H, m), 8.10 (2H, dd, J=1.6 and 4.6 Hz) and 8.83 (2H, dd, J=1.6 and 4.6 Hz).

### 4-(Benzoxazol-2-yl)-1-hexylpyridin-1-ium iodide

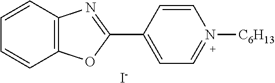

A solution of 2-(pyridin-4-yl)benzoxazole (1.00 g, 5.1 mmol) and 1-iodohexane (3.24 g, 15.3 mmol) in MeCN (30 mL) was heated at reflux in the dark under N₂ with stirring. After 16 h, the resulting mixture was cooled and diluted with Et₂O (50 mL). The precipitate was filtered off, washed with Et₂O (3×10 mL) and air dried to give the title compound (2.06 g, 99%) as a yellow powder.

### Compound 22: 4-(Benzoxazol-2-yl)-1-hexylpyridin-1-ium tetrafluoroborate

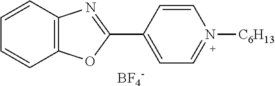

A solution of 4-(benzoxazol-2-yl)-1-hexylpyridin-1-ium iodide (2.01 g, 4.9 mmol) in warm MeOH/water (50 mL, 1:1) was added dropwise to a solution of NaBF₄ (5.42 g, 40 mmol) in water (100 mL) with stirring. Stirring was continued for 0.5 h and the resulting precipitate filtered off, washed with water (3×10 mL) and air dried to give the title compound (1.77. g, 98%) as a colourless powder, δ_{H} (CD₃OD, 400 MHz) 0.89-0.97 (3H, bt, J=5.7 Hz), 1.30-1.51 (6H, m), 2.00-2.14 (2H, m), 4.69 (2H, bt, J=7.4 Hz), 7.50-7.65 (2H, bm), 7.83 (1H, bd, J=8.2 Hz), 8.93 (1H, bd, J=8 Hz), 8.78 (2H, bd, J=5.5 Hz) and 9.16 (2H, bd, J=5.5 Hz).

### Example 23

### 4-(Benzoxazol-2-yl)-1-phenylpyridin-1-ium triflate

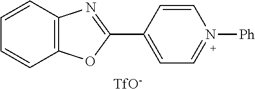

A mixture of 2-(pyridin-4-yl)benzoxazole (1 g, 5.1 mmol), diphenyliodonium triflate (3.29 g, 7.6 mmol), Cu(OAc)₂·H₂O (100 mg, 10 mol %) in dry DMF (50 mL) was heated at 100° C. for 16 h, cooled and the solvent removed under reduced pressure. The residue was triturated with Et₂O (50 mL), washed with Et₂O (3×10 mL) and air dried. The resulting solid was triturated with hot MeOH (20 mL), cooled, filtered and air dried to give the title compound (1.86 g, 86%) as a colourless powder, δ_{H} (CD₃OD, 400 MHz) 7.54-7.70 (2H, bm), 7.76-8.00 (7H, bm), 8.93 (2H, bd, J=5.9 Hz) and 9.42 (2H, bd, J=5.9 Hz).

### Compound 23: 4-(Benzoxazol-2-yl)-1-phenylpyridin-1-ium tetrafluoroborate

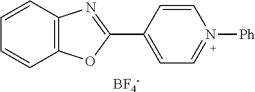

A solution of 4-(benzoxazol-2-yl)-1-phenylpyridin-1-ium triflate (0.25 g, 0.59 mmol) in warm MeOH (10 mL) was added dropwise to a solution of NaBF₄ (0.65 g, 5.9 mmol) in water (30 mL) with stirring. The resulting precipitate was filtered, washed with water (2×3 mL), dissolved in warm MeOH (10 mL) and added dropwise to a solution of NaBF₄ (0.65 g, 5.9 mmol) in water (30 mL) and stirred for 0.5 h. The resulting precipitate was filtered off, washed with water (2×3 mL) and air dried to give the title compound (0.20 g, 95%) as a pale yellow powder, δ_{H} (DMSO-d₆, 400 MHz) 7.56-7.62 (1H, m), 7.64-7.70 (1H, m), 7.74-7.82 (3H, m), 7.91-8.01 (3H, m), 8.05 (1H, d, J=7.9 Hz), 8.87 (2H, bd, J=6.8 Hz) and 9.53 (2H, bd, J=6.8 Hz).

### Example 24

### 4-(2-Methoxybenzoyl)pyridine

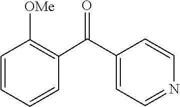 [SMILES: COc1ccccc1C(=O)c1ccncc1]

A freshly prepared solution of 2-methoxyphenylmagnesium bromide (40 mL, ca. 1.38 M, 55.4 mmol) was added to a stirred solution of 4-cyanopyridine (2.78 g, 26.7 mmol) in dry THF (20 mL) which had been cooled to 0° C. under N₂. After the addition was complete the reaction mixture was stirred and heated at 50° C. under N₂ for 16 h. The solution was then cooled to 0° C. and H₂O (20 mL) was slowly added. The reaction mixture was then evaporated to dryness under reduced pressure and then 2M HCl (100 mL) was added, and the solution was heated at 80° C. for 8 h. The reaction mixture was then cooled to room temperature and the solution was washed with EtOAc (2×100 mL). The aqueous phase was basified with NaOH (5 M) and extracted with DCM (3×100 mL). The organic layers were combined, dried (Na₂SO₄), filtered and the solvent was removed under reduced pressure. The resulting residue was chromatographed on silica gel [eluent=1:9 graduated to 1:1 EtOAc:Petroleum ether]. The solvent of the resulting column fractions was removed under reduced pressure to give the title compound as a yellow oil. Yield 4.60 g, 82%. δ_{H} (CDCl₃, 400 MHz) 8.75 (2H, dd, J=4.4, 1.6 Hz), 7.57-7.51 (3H, m), 7.46 (1H, dd, J=7.6, 1.8 Hz), 7.07 (1H, td, J=7.55, 0.9 Hz), 6.99 (1H, d, J=8.3 Hz) and 3.68 (3H, s); δ_{C} (CDCl₃, 100 MHz) 195.6, 158.0, 150.5, 144.8, 133.5, 130.5, 127.3, 122.4, 121.0, 111.7 and 55.6.

### 3-(Pyridin-4-yl)-1,2-benzisoxazole

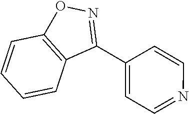 [SMILES: c1ccc2c(-c3ccncc3)noc2c1]

4-(2-Methoxybenzoyl)pyridine (3.60 g, 16.8 mmol) was dissolved in dry DCM (100 mL) and cooled to 0° C. under N₂. BBr₃ (8.42 g, 3.24 mL, 33.6 mmol) was added dropwise and the reaction mixture was stirred for 16 h. The reaction mixture was added to ice, neutralized with NaHCO₃ and stirred for 1 h. The reaction mixture was then extracted with DCM (2×200 mL) and the organic layers were combined, dried (Na₂SO₄), filtered, and the solvent removed under reduced pressure. The resulting residue was chromatographed on silica gel [eluent=1:9 graduated to 4:6 EtOAc:Petroleum ether]. The solvent of the resulting column fractions was removed under reduced pressure to give impure 4-(2-hydroxybenzoyl)pyridine (2.60 g) as a yellow solid which was used in the subsequent reactions without further purification. The impure 4-(2-hydroxybenzoyl)pyridine (2.60 g) was dissolved in 7N ammonia in methanol (30 mL) and stirred at room for 48 h to give an orange coloured solution. The reaction mixture was then evaporated to dryness under reduced pressure and redissolved in dry THF (40 mL) under N₂. N-Chlorosuccinimide (2.60 g, 19.5 mmol) and K₂CO₃ (3.60 g, 138 mmol) were then added to the reaction mixture which was then stirred for 16 h at room temperature under N₂. The reaction mixture was then diluted with diethyl ether (100 mL) and quenched with water (100 mL). The organic layer was separated and the aqueous layer was extracted with diethyl ether (2×100 mL). The organic layers were combined, dried (Na₂SO₄), filtered and the solvent removed under reduced pressure. The resulting residue was chromatographed on silica gel [eluent=1:9 graduated to 3:7 EtOAc:petroleum ether]. The solvent of the resulting column fractions was removed under reduced pressure to give a pale yellow solid that was then triturated with pentane and dried under reduced pressure to give the title compound as an off-white solid. Yield 1.2 g, 36%. δ_{H} (CDCl₃, 400 MHz) 8.84 (2H, dd, J=4.5, 1.5 Hz), 7.95 (1H, dt, J=8.0, 0.9 Hz), 7.89 (2H, dd, J=4.4, 1.6 Hz) 7.70 (1H, dt, J=8.4, 0.8 Hz), 7.67 (1H, ddd, J=8.5, 7.0, 1.0 Hz) and 7.44 (1H, ddd, J=8.5, 7.0, 1.0 Hz); δ_{C} (CDCl₃, 100 MHz) 164.3, 155.4, 150.9, 136.7, 130.4, 124.6, 122.3, 1217, 119.9, 110.6.

### Compound 24: 4-(1,2-Benzisoxazol-3-yl)-1-phenylpyridin-1-ium hexafluorophosphate

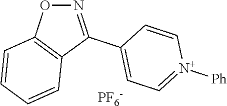

3-(Pyridin-4-yl)-1,2-benzisoxazole (1.00 g, 5.1 mmol) diphenyliodonium trifluoromethanesulfonate (3.29 g, 7.64 mmol) and Cu(OAc)₂. H₂O (0.1 g, 0.51 mmol) were dissolved in DMF (40 mL) under N₂ and the reaction mixture was stirred at 100° C. for 16 h. The reaction mixture was cooled to room temperature and the solvent was removed under reduced pressure. The resulting pale green solid was triturated with diethyl ether (3×50 mL), filtered and dried under reduced pressure. The resulting hydroscopic pale green solid was dissolved in hot MeOH:H₂O (ca. 2:1, 150 mL) and added dropwise through a cotton wool plug to a stirred solution of NH₄PF₆ (17.5.0 g) in H₂O (350 mL) whereupon a pale yellow precipitate formed. The suspension was stirred for 30 minutes and then filtered under reduced pressure. The resulting solid was washed with water (50 mL) and then dried under reduced pressure to give a pale green powder. The solid was then dissolved in hot acetone:H₂O (ca. 2:1, 200 mL) and added dropwise through a cotton wool plug back into the filtrate (which had been reduced by ca. 20% under vacuum) to give a pale green precipitate. The resulting suspension was then filtered under reduced pressure and the solid was washed with water (50 mL) and then dried under reduced pressure to give a pale green powder. The green powder was triturated with MeOH (50 mL), filtered and dried under reduced pressure to give the title compound as a colourless powder. Yield 1.17 g, 55%. δ_{H} (DMSO-d₆, 400 MHz) 9.60 (2H, d, J=7.0 Hz), 8.96 (2H, d, J=6.9 Hz), 8.43 (1H, d, J=8.1 Hz), 8.09 (1H, d, J=8.6 Hz) 8.05-7.98 (2H, m), 7.93 (1H, ddd, J=8.5, 7.1, 1.0 Hz), 7.89-7.79 (3H, m) and 7.72 (1H, ddd, J=8.5, 7.3, 0.5 Hz); δ_{C} (DMSO-d₆, 100 MHz) 164.1, 153.3, 146.0, 144.1, 142.5, 131.6, 131.5, 130.3, 126.2, 125.8, 124.8, 122.4, 118.6 and 110.7; δ_{F} (DMSO-d₆, 376 MHz) −170.13 (6F, d, J=711.0 Hz).

### Evaluation of Oxido-Reduction Potentials and Absorption Spectra of the Compounds of the Invention

### Method for Measuring Oxido-Reduction Potentials

The oxido-reduction potentials of the compounds are measured by cyclic voltammetry with 3 electrodes.

The 3 electrodes used are:
1 Platinum working electrode
1 Platinum auxiliary or counter electrode
1 Platinum reference electrode which is immersed into a solution constituted of 0.01 M AgNO₃+0.1 M TBAP (tetrabutylammonium perchlorate) in acetonitrile.

The scan rate of the potential is fixed to 100 mV/s.

E₁^{red} corresponds to the first reduction peak of the analyzed compound.

E₂^{red} corresponds to the second reduction peak of the analyzed compound.

E₁^{1/2} corresponds to the oxido-reduction potential of an oxidant/reductor system as calculated below:

E₁^{1/2}=(E₁^{red}+E₁^{ox})/2

wherein E₁^{ox} corresponds to the first oxidation peak of the analyzed compound.

ΔE^{red} corresponds to the difference between E₁^{red} and E₂^{red} as calculated below: ΔE_{red}=|E₂^{red}|−|E₁^{red}|.

The indicated potential values are the first reduction potentials for the compounds, with respect to the standard hydrogen reference electrode (SHE).

The analyzed solution comprises 0.005 M of the compound to be analyzed and 0.25 M of TBABF₄ salt in propylene carbonate as solvent.

### Method for Measuring Absorption Spectra

This solution is introduced into a quartz cell where.

This solution is introduced into a quartz cell where at least one working electrode in the form of a platinum grid is placed to colour the analysed compound on this electrode. The absorption spectrum of the analysed compound in the time domain is measured by uv-visible spectroscopy.

The results for each of the synthesized compounds are indicated in Table 1 below. E₁^{red} corresponds to the first reduction potential. The colour indicated in Table 1 is the visual colour perceived by emmetropic eyes under day light conditions. It should be noted that the λₘₐₓ value just gives an approximate indication of the colour of a particular compound. However, as a consequence of the broad nature of the absorption bands, the whole absorption spectrum has to be taken into account in order to understand the final perceived colour of any one compound.

In comparison, Table 2 shows the results obtained for 3 known compounds.

By comparing compound 2 (orange) and compounds 6-7 (red) of the invention with known compound COMP1 (green), it appears that replacing one phenyl pyridinium group by one substituted benzimidazolium or substituted benzothiazolium group reduces the maximum absorption wavelength in the visible range from 645 nm to 550 nm or below. At their activated state these molecules are red or orange rather than green.

By comparing compounds 1, 10, 16 (orange), 3, 4, 5 (purple), 8 (green), 9, 11 (yellow/green), 13-14 (yellow), with known compounds COMP2 (blue) and COMP3 (blue), it appears that including different groups like imidazolium, benzimidazolium, benzothiazolium, alone or included in more complex molecular structures have also this effect of shifting the maximum absorption wavelength to lower values. These groups can be either introduced in between two alkyl bi pyridinium groups or could replace the central pyridinium group of a ter pyridinium. The molecules thus obtained are yellow, orange, red, green or purple rather than blue.

The results show that the known compound COMP3, which contains a phenyl group in between two alkyl bi pyridinium groups, is also orange with an activation potential of −1.21V. The compounds of the invention have a similar or lower activation potential than COMP3, except compounds 11 and 14.

TABLE 1

| | | | | | | | | | E₁^{red} | E1 peak | E₂^{red} | E2 peak | |
|---|---|---|---|---|---|---|---|---|---|---|---|---|---|
| | | | E₁ ^{1/2} | | | λ1 | λ2 | λ cut | peak | potential | peak | potential | |
| | | E₁ ^{1/2} | (V) | reversi- | | max | max | (nm) | potential | (V) vs | potential | (V) vs | |
| Cpd | Structure | (V) | vs SHE | bility | color | (nm) | (nm) | clear | (V) | SHE | (V) | SHE | ΔE^{red} |
| 1 | 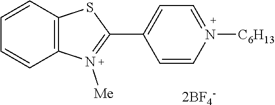 | −0.62 | −0.078 | Y | orange | 421 | 530 | 399 | −0.65 | −0.108 | −0.92 | −0.378 | −0.27 |
| 2 | 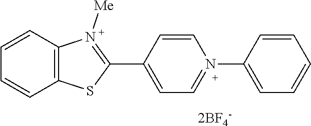 | −0.53 | 0.012 | Y | orange | 446 | 550 | 435 | −0.57 | −0.028 | −0.78 | −0.238 | −0.21 |
| 3 | 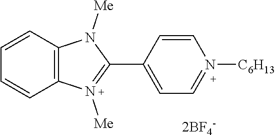 | — | NA | N | purple | 390 | wide absorb- tion | 380 | −1 | −0.458 | — | NA | NA |
| 4 | 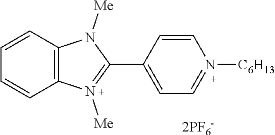 | −0.98 | −0.438 | Y | purple | 390 | 525 | 382 | −1 | −0.458 | −1.35 | −0.808 | −0.35 |
| 5 | 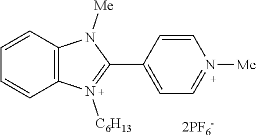 | — | NA | N | purple | 402 | 530 | 373 | −0.48 | 0.062 | −0.86 | −0.318 | −0.38 |
| 6 | 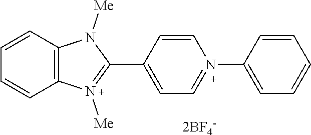 | −0.8 | −0.258 | Y | red | 416 | 530 | 397 | −0.87 | −0.328 | −1.22 | −0.678 | −0.35 |
| 7 | 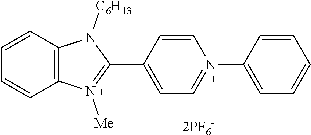 | −0.82 | −0.278 | Y | red | 415 | 530 | 393 | −0.9 | −0.358 | −1.24 | −0.698 | −0.34 |
| 8 | 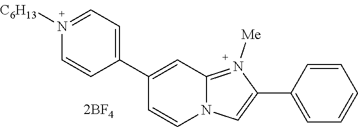 | −1.07 | −0.528 | Y | green | 437 | 650 | 473 | −1.1 | −0.558 | −1.5 | −0.958 | −0.4 |
| 9 | 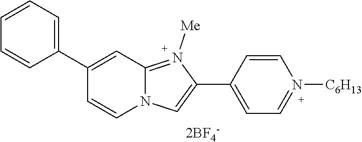 | −1.2 | −0.658 | Y | yellow | 412 | — | 412 | −1.24 | −0.698 | −1.65 | −1.108 | −0.41 |
| 10 | 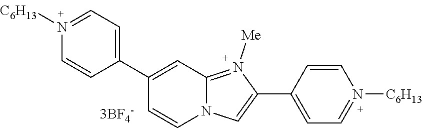 | −0.96 | −0.418 | Y | orange | 470 | 580 | | | 0.542 | | 0.542 | 0 |
| 11 | 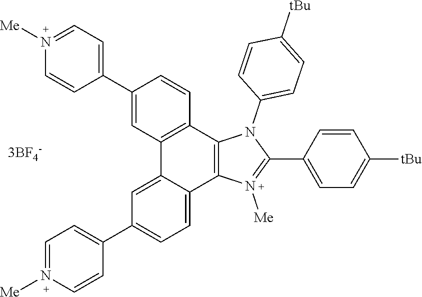 | −1.39 | −0.848 | Y | green | 410 | 640 | 450 | −1.45 | −0.908 | — | NA | NA |
| 12 | 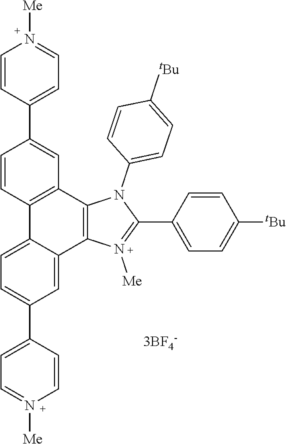 | −1.27 | −0.728 | Y | orange | 486 | 750 | 439 | −1.32 | −0.778 | — | NA | NA |
| 13 | 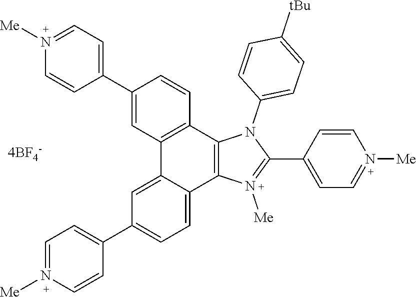 | | 0.542 | N | yellow | 443 | 650 | 456 | −0.98 | −0.438 | −1.48 | −0.938 | −0.5 |
| 14 | 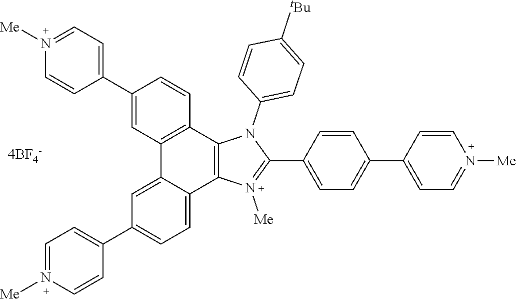 | −1.4 | −0.858 | Y | yellow | 409 | 750 | 426 | −1.53 | −0.988 | — | NA | NA |
| 15 | 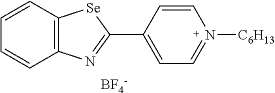 | −1.12 | −0.578 | Y | red | 533 | | 437 | −1.15 | −0.608 | — | NA | NA |
| 16 | 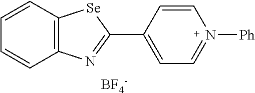 | −0.94 | −0.398 | Y | red | 541 | | 458 | −1.02 | −0.478 | — | NA | NA |
| 17 | 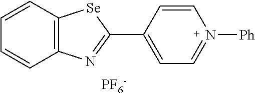 | −1 | −0.458 | Y | red | 545 | | 463 | −1 | −0.458 | — | NA | NA |
| 18 | 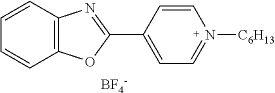 | −1.14 | −0.598 | Y | orange | 405 | 496 | 407 | −1.17 | −0.628 | — | NA | NA |
| 19 | 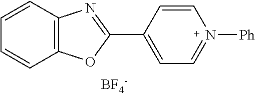 | −0.97 | −0.428 | Y | orange | 405 | 495 | 425 | −1 | −0.458 | — | NA | NA |

TABLE 2

| | | | E₁ ^{1/2} | | | | |
|---|---|---|---|---|---|---|---|
| | | | (V) | | | λ1 | λ2 |
| | | E₁ ^{1/2} | vs N | reversi- | | max | max |
| Ref | Structure | (V) | SHE | bility | color | (nm) | (nm) |
| COMP 1 | 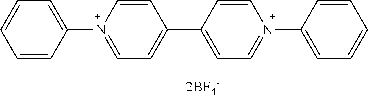 | −0.62 | −0.078 | Y | green | 442 | 645 |
| COMP 2 | 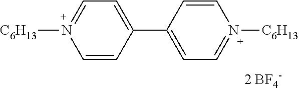 | −0.79 | −0.248 | Y | blue | 399 | 608 |
| COMP 3 | 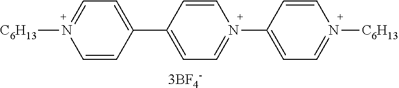 | −0.41 | 0.132 | Y | blue | 400 | 620 |
| | | | E₁^{red} | E1 peak | E₂^{red} | E2 peak | |
| | | λ cut | peak | potential | peak | potential | |
| | | (nm) | potential | (V) vs | potential | (V) vs | |
| | Ref | clear | (V) | SHE | (V) | SHE | ΔE^{red} |
| | COMP 1 | 395 | −0.64 | −0.098 | −0.93 | −0.388 | −0.29 |
| | COMP 2 | 316 | −0.84 | −0.298 | −1.2 | −0.658 | −0.36 |
| | COMP 3 | 410 | −0.47 | 0.072 | −0.69 | −0.148 | −0.22 |

1: An electrochromic compound represented by formula (I): 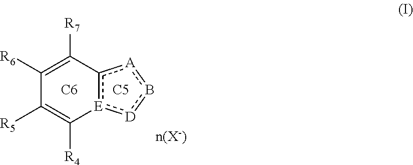
wherein:
A is N, ⁺N, N—R₁, ⁺N—R₁ or C—R₁;
B is C—R₂, S, Se, O, N, N—R₂ or ⁺N—R₂;
D is C—R₃, N, S, O, Se, N—R₃ or ⁺N—R₃;
E is C, N or ⁺N;
R₁ is H, C₁-C₁₈ alkyl, aryl or Z;
R₂ is H, C₁-C₁₈ alkyl, aryl, Z or aryl substituted by Z;
R₃ is H or C₁-C₁₈ alkyl, aryl or Z;
R₄ is H, C₁-C₁₈ alkyl, aryl or Z;
R₅ is H, C₁-C₁₈ alkyl, aryl or Z;
R₆ is H, C₁-C₁₈ alkyl, aryl or Z;
R₇ is H, C₁-C₁₈ alkyl, aryl or Z;
R₇ and R₆ and/or R₆ and R₅ and/or R₅ and R₄ may form together an aromatic ring or heteroaromatic ring fused to the six-membered(hetero)cyclic core (Cycle C6) they are attached to, optionally substituted by Z, 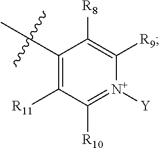
With Z is
Y is C₁-C₁₈ alkyl, (hetero)aryl or (hetero)arylalkyl;
R₈, R₉, R₁₀ and R₁₁ are independently selected from H and C₁-C₁₈ alkyl;
R₈ and R₉ or R₁₀ and R₁₁ may form an aromatic ring fused to the pyridium group they are attached to,
When B=C—Z, and when A=⁺N, R₈ or R₁₁ may form with A a ring, aromatic or not, fused with the five-membered heterocyclic ring (Cycle C5) A is attached to,
n is selected to counterbalance the number of positive charges;
X is a counterion; [symbol]
is a single bond or a double bond;
with the 3 following provisos:
1) Cycle C5 is a five membered heterocyclic ring with 2 of A, B, D and E being independently selected from: N, N—R₁, ⁺N—R₁, N—R₂, ⁺N—R₂, ⁺N—R₃, ⁺N—R₃, S, Se and O;
2) Cycle C5 and Cycle C6 form a conjugated system; and
3) at least one of R₁, R₂, R₃, R₄, R₅, R₆ or R₇ is Z or at least R₇ and R₆ form together an aromatic ring substituted by Z or at least R₅ and R₆ form together an aromatic ring substituted by Z or at least R₅ and R₄ form together an aromatic ring substituted by Z.

2: The compound of formula (I) according to claim 1, wherein said compound is represented by formulae (II), (III), (IV), (V), (VI), (VII), (VIII), (IX) or (X): 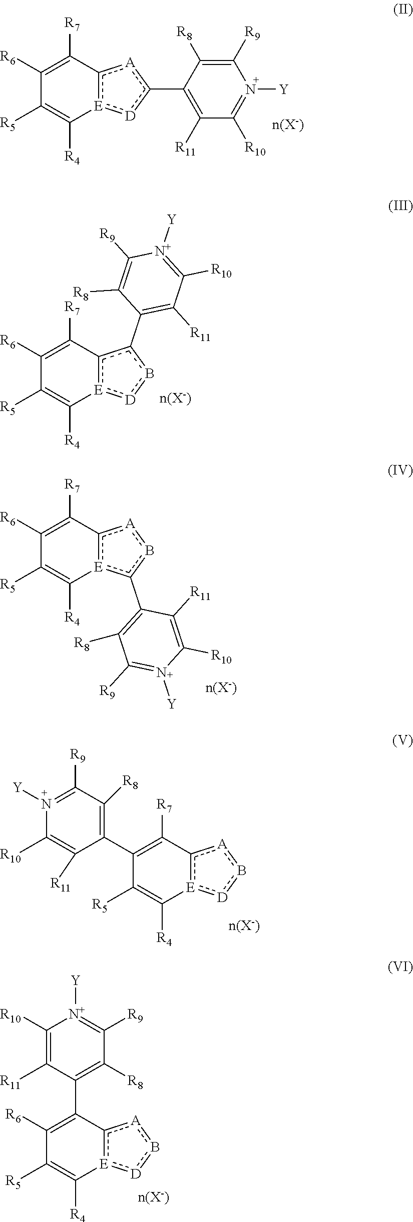 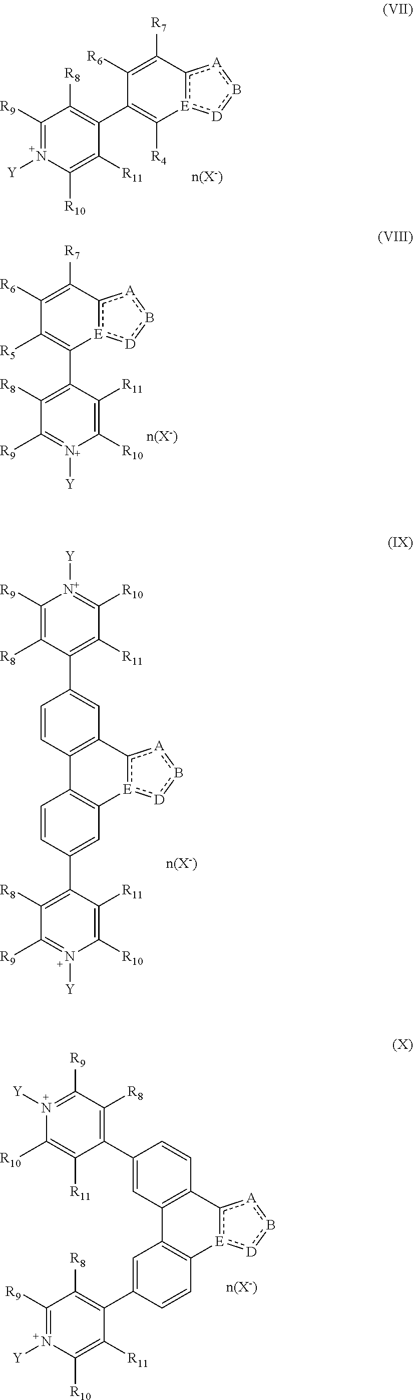
wherein A, B, D, E, R₁, R₂, R₃, R₄, R₅, R₆, R₇, R₈, R₉, R₁₀ and R₁₁, when present, are as defined in claim 1;
and wherein Z, Y, n and X are also as defined in claim 1.

3: The compound of formula (I) according to claim 1 wherein
A is N, ⁺N, N—R₁ or ⁺N—R₁;
and/or B is N, N—R₂ or ⁺N—R₂;
and/or D is N, N—R₃ or ⁺N—R₃.

4: The compound of formula (I) according to claim 1 wherein E is N or ⁺N and/or D is S.

5: The compound of formula (II) according to claim 2 wherein
A is N, ⁺N, N—R₁ or ⁺N—R₁;
D is N—R₃, ⁺N—R₃, S, Se, or O;
E is C.

6: The compound of formula (II) according to claim 5 wherein
A is N⁺ or ⁺N—R₁;
D is S; and
E is C.

7. The compound of formula (II) according to claim 6 wherein
A is ⁺N;
D is S;
E is C and;
R₈ forms with A, a five or six-membered ring, unsaturated or saturated, fused with Cycle C5 A is attached to and is represented by formula (XI), (XII) or (XIII).

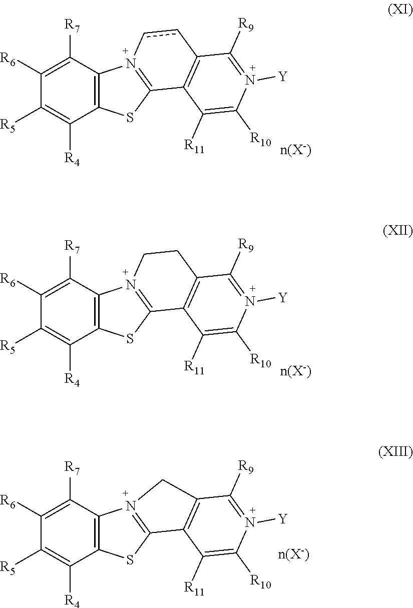

8: The compound of formula (II) according to claim 6 wherein
A is ⁺N—R₁;
D is S; and
E is C.

9: The compound of formula (II) according to claim 8 wherein
R₇ and R₆ and/or R₆ and R₅ and/or R₅ and R₄ form together an aromatic ring or heteroaromatic ring fused to the (hetero)cyclic core (Cycle C6) they are attached to, optionally substituted by Z.

10: The compound of formula (II) according to claim 8 wherein
R₈ and R₉ or R₁₀ and R₁₁ form an aromatic ring fused to the pyridium group they are attached to.

11: The compound of formula (I) according to claim 1, wherein said compound is selected from the group consisting of:

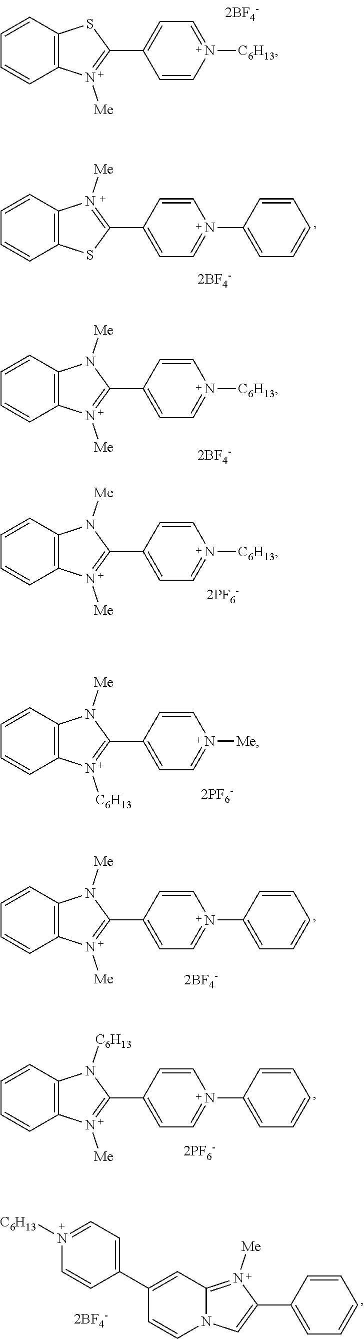

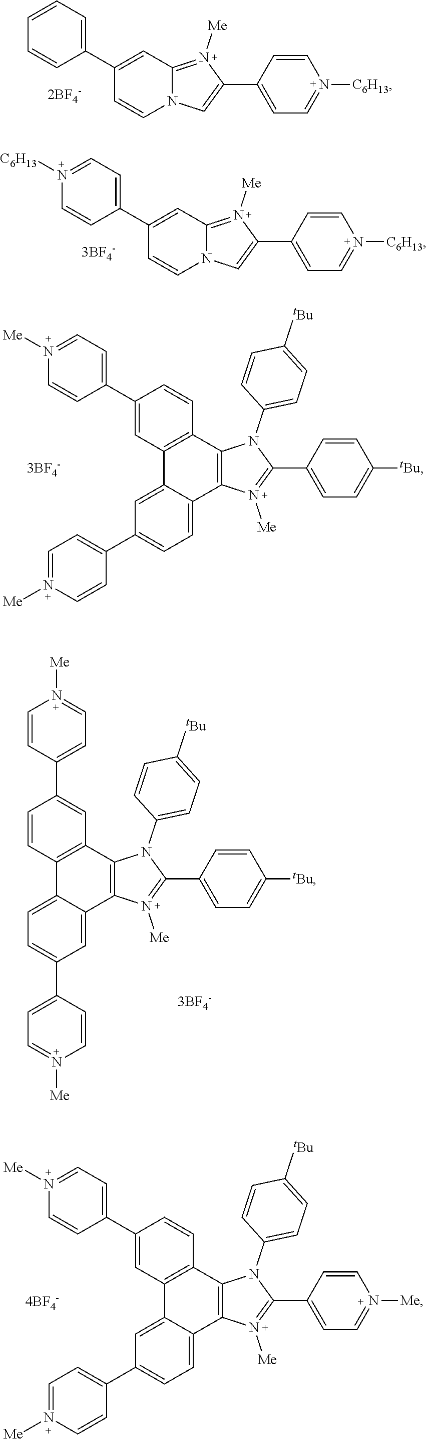

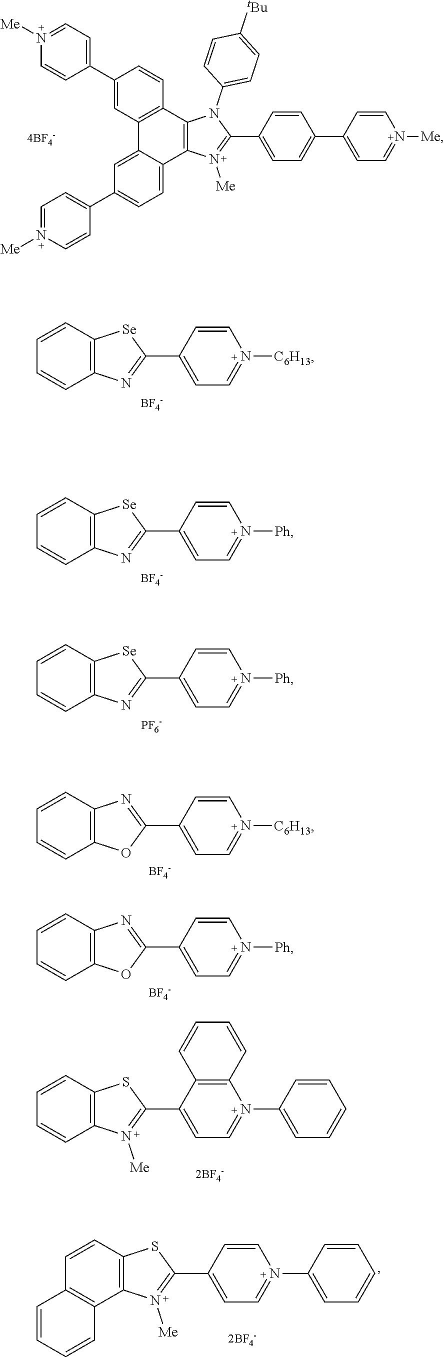

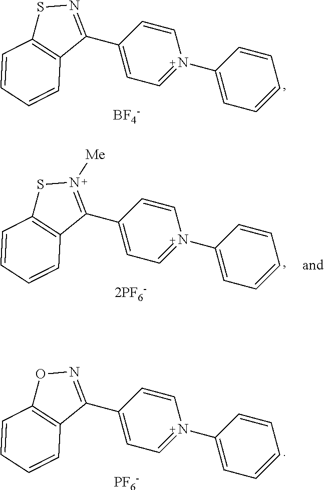

12: An electrochromic composition comprising at least one compound as defined in claim 1.

13: The electrochromic composition according to claim 12, wherein said composition further comprises a host medium, a mesomorphous media or a gel.

14: An electrochromic device comprising a compound as defined in claim 1.

15: The electrochromic device according to claim 14, wherein said electrochromic device is selected from an optical article, a window, a visor, a mirror, a head mounted device and a display.

16: An electrochromic device comprising an electrochromic composition as defined in claim 12.

17: The electrochromic device according to claim 16, wherein said electrochromic device is selected from an optical article, a window, a visor, a mirror, a head mounted device and a display.
